(19) United States
(12) Patent Application Publication
(10) Pub. No.: US 2020/0370070 A1
(43) Pub. Date: Nov. 26, 2020

(54) COMPOSITIONS AND METHODS FOR EFFICIENT GENOME EDITING

(71) Applicant: THE JOHNS HOPKINS UNIVERSITY, Baltimore, MD (US)

(72) Inventors: Geraldine Seydoux, Chevy Chase, MD (US); Alexandre Paix, Saint Raphael (FR)

(21) Appl. No.: 16/763,827

(22) PCT Filed: Nov. 19, 2018
(86) PCT No.: PCT/US2018/061770

§ 371 Date: May 13, 2020

Related U.S. Application Data
(60) Provisional application No. 62/587,554, filed on Nov. 17, 2017.

(51) Int. Cl. C12N 15/90; C12N 15/873; C12N 15/88

(52) U.S. Cl. CPC C12N 15/907; C12N 15/88; C12N 15/873

(57) ABSTRACT

The present invention relates to the field of genome editing. More specifically, the present invention provides compositions and methods useful in clustered regularly interspaced short palindromic repeats (CRISPR)-based techniques. In one embodiment, the present invention provides a double-stranded, linear donor polynucleotide comprising a template polynucleotide flanked by a first homology arm and a second homology arm, wherein the homology arms are between 30-35 bases in length.

### CROSS-REFERENCE TO RELATED APPLICATIONS

This application claims the benefit of U.S. Provisional Application No. 62/587,554, filed Nov. 17, 2017, which is incorporated herein by reference in its entirety.

### FIELD OF THE INVENTION

The present invention relates to the field of genome editing. More specifically, the present invention provides methods and compositions useful in the design of synthetic donor DNAs for efficient genome editing.

### BACKGROUND OF THE INVENTION

Precision genome editing begins with the creation of a double-strand break (DSB) in the genome near the site of the desired DNA sequence change (“edit”) (Jasin, M. & Haber, J. E., 44 DNA REPAIR (AMST.) 6-16 (2016)). Generation of targeted DSBs has been greatly accelerated in recent years by the discovery of CRISPR-Cas9, a programmable DNA endonuclease that can be targeted to a specific DNA sequence by a small “guide” RNA (crRNA) (Doudna, J. A. & Charpentier, E., 346(6213) SCIENCE 1258096 (2014)). DSBs are lethal events that must be repaired by the cell's DNA repair machinery. DSBs can be repaired via imprecise, non-homology-based repair mechanisms, such as non-homologous end-joining (NHEJ), or by precise, homology-dependent repair (HDR) (Danner et al., 28(7-8) MAMMALIAN GENOME 262-74 (2017)). HDR utilizes DNAs that contain homology to sequences flanking the DSB (termed homology arms) to template the repair. If a synthetic “donor” DNA containing the desired edit is available when the DSB is generated, the cellular HDR machinery will use the donor DNA to repair the DSB and the edit will be incorporated at the targeted locus (Jasin & Haber (2016)). Several studies have reported that single-stranded oligonucleotides (ssODNs) can be used to introduce short edits (<50 bases) (Liang et al., 241 J. BIOTECHNOL. 136-46 (2016)). ssODNs that target the DNA strand that is first released by Cas9 after DSB generation have been reported to perform best (Richardson et al., 34(3) NAT. BIOTECHNOL. 339-44 (2016)). This strand preference, however, has only been tested for small edits near the DSB and has not been noticed at all loci (Liang et al. (2016)). Edits at a distance from the DSB (>10 bp) are recovered at lower frequencies (Liang et al. (2016; Paquet et al., 533(7601) NATURE 125-29 (2016)). Recovery of large edits (such as GFP knock-ins) has also been reported to be inefficient, requiring large plasmid donors with long (>500 nt) homology arms or selection markers to recover the rare edits (Danner et al. (2017)). Large insertions have been obtained through non-homologous or micro-homology-mediated end joining reactions (NHEJ and MMEJ), but these approaches require simultaneous Cas9-induced cleavage of donor and target DNAs. See Yao et al., 20 EBIO MEDICINE 19-26 (2017); Yao et al., 27(6) CELL RES. 801-14 (2017); Zhang et al., 18(1) GENOME BIOL. 35 (2017); He et al., 44(9) NUCLEIC ACIDS RES. e85 (2016); Suzuki et al., 540(7631) NATURE 144-49 (2016); Yamamoto et al., 5(9) G3 (BETHESDA) 1843-47 (2015); Nakade et al., 5 NAT. COMMUN. 5560 (2014). Thus, there exists a need for more efficient genome editing tools and techniques.

### SUMMARY OF THE INVENTION

Genome editing, the introduction of precise changes in the genome, is revolutionizing our ability to decode the genome. The present invention is based, at least in part, on the development of compositions and methods for genome editing in mammalian cells that uses linear, double-stranded donor DNAs to introduce precise changes in the genome. As described herein, the present inventors demonstrate that PCR fragments containing edits up to 1 kb require only about 35 bp homology arms to initiate Cas9-induced double-strand breaks in human cells and mouse embryos. In addition, the present inventors have developed donor DNA design rules that maximize the recovery of edits without cloning or selection.

Accordingly, in one aspect, the present invention provides compositions useful for more efficient genome editing. In one embodiment, the present invention provides a double-stranded, linear donor polynucleotide comprising a polynucleotide encoding a fluorescent protein flanked by a first homology arm and a second homology arm. In a specific embodiment, the homology arms are 15-60 bases in length. In a more specific embodiment, the homology arms are 25-45 bases in length. In an even more specific embodiment, the homology arms are 30-40 bases in length.

In another embodiment, the present invention provides a double-stranded, linear donor polynucleotide comprising a polynucleotide encoding a fluorescent protein flanked by a first homology arm and a second homology arm, wherein the first and second homology arms are between 30-35 bases in length.

A double-stranded, linear donor polynucleotide can comprise a template polynucleotide encoding an edit flanked by an intervening sequence and two homology arms. In one embodiment, the homology arms are 15-60 bases in length. In another embodiment, the homology arms are 25-45 bases in length. In an alternative embodiment, the homology arms are 30-40 bases in length. In certain embodiments, the template polynucleotide is up to 1 kb in length. The template polynucleotide can comprise a sequence designed to change at least one nucleotide base within 30 bases of a double-stranded break (DSB) of a target nucleic acid. In another embodiment, the template polynucleotide further comprises a restriction enzyme site.

In an alternative embodiment, a double-stranded, linear donor polynucleotide comprises a template polynucleotide flanked by a first homology arm and a second homology arm, wherein the homology arms are between 30-35 bases in length. In particular embodiments, the template polynucleotide is up to 1 kb in length. In a specific embodiment, the template polynucleotide comprises a sequence designed to change at least one nucleotide base within 30 bases of a DSB of a target nucleic acid. In another embodiment, the template polynucleotide further comprises a restriction enzyme site.

In another aspect, the present invention provides methods for more efficient genome editing. In one embodiment, a method comprises the step of performing a clustered regularly interspaced short palindromic repeats (CRISPR)-based technique using a double-stranded, linear donor polynucleotide described herein as the donor polynucleotide. In another embodiment, the present invention provides a method comprising injecting into a target cell a composition comprising (a) an RNA-guided DNA endonuclease; (b) a guide RNA; and (c) a double-stranded, linear donor polynucleotide described herein.

### BRIEF DESCRIPTION OF THE FIGURES

FIG. 1A-1B. Tagging of the mouse Adcy3 locus with mCherry using a PCR donor with short homology arms. FIG. 1A: schematic representation of the mouse Adcy3 locus repair strategy using a PCR donor: mCherry (red), Homology arm Sequences (HS, blue), locus (grey lines), DSB (blue line). FIG. 1B: agarose gel showing representative PCR reactions using primers flanking the DSB at the Adcy3 locus (primers correspond to sequence outside the HS from the PCR donor). The upper bands (‘insert’ arrow) correspond to the mCherry insertion.

FIG. 2A-2D. PCR fragments with short homology arms are efficient donors to create GFP knock-ins in HEK293T cells. FIG. 2A: diagrams showing PCR donors for GFP insertion at the Lamin A/C and RAB11A loci. Locus—grey, GFP—green, HS (Homology arm Sequences)—blue, DSB—vertical line. GFP was inserted at the DSB in Lamin A/C and 11 bp upstream of the DSB in RAB11A. FIG. 2B: graphs showing % of GFP+ cells obtained with PCR donors with HS of the indicated lengths (33/33 refers to a right HS and a left HS, each 33 bp long). Insert size in all cases was 714 bp. Each bar represents the average insertion efficiency from two or more independent experiments (Table 1). Error bars represent the +/−SD. PCR fragments were nucleofected in HEK293T cells at the concentration indicated and were counted by flow cytometer 3 days later. See Table 1. FIG. 2C: graphs showing % of GFP+ cells obtained with PCR or plasmid donors with HS of the indicated lengths. Insert size in all cases was 714 bp. Each bar represents the average insertion efficiency from two or more independent experiments (Table 1). Error bars represent the +/−SD. PCR fragments were nucleofected in HEK293T cells at the concentration indicated and cells were counted by flow cytometer 3 days later. FIG. 2D: confocal images of cells 3 days after nucleofection. GFP: green, DNA: blue. The GFP subcellular localizations are as expected for in frame translational fusions.

FIG. 3A-3C. Editing efficiency increases with decreasing insert size. The graphs show % of GFP+ cells obtained with PCR donors with HS and inserts of the indicated lengths. Each bar represents the average insertion efficiency from two or more independent experiments (Table 1). Error bars represent the +/−SD. FIG. 3A: knock-in of donors containing full-length GFP at the Lamin A/C locus. PCR fragments were nucleofected in HEK293T cells at the concentration indicated and cells were counted by microscopy 3 days later. FIG. 3B: knock-in of donors containing full-length GFP or GFP11 at the Lamin A/C locus. PCR fragments were nucleofected at the concentration indicated in HEK293T (expressing GFP1-10) and cells were counted by microscopy 3 days later. FIG. 3C: knock-in of donors containing full-length GFP or GFP11 at the RAB11A locus (11 bp upstream of DSB). PCR fragments were nucleofected at the concentration indicated in HEK293T (expressing GFP1-10) and cells were counted by flow cytometer 3 days later.

FIG. 4A-4C. Repair is a polarity-sensitive process. FIG. 4A: synthesis-dependent strand annealing (SDSA) model for gene conversion. In this, and all other schematics, each line corresponds to a DNA strand. Locus DNA is in grey, donor homology arms are in blue, donor insert is in green, and arrows indicate 3′ ends. Donor DNA strands of opposite polarity are shown above and below the locus for clarity. PCR donors contain both strands, ssODNs donors would contain either a sense or antisense strand. Dotted lines represent DNA synthesized during the repair process. Resection of DSB: DSB is resected creating 3′ overhangs on each side of the DSB. Strand invasion and DNA synthesis: The overhangs pair with complementary strands in the donor and are extended by DNA synthesis. Annealing: The newly synthesized strands withdraw from the donor and anneal back at the locus. Ligation (not shown) seals the break. FIG. 4B: diagrams showing donor ssODNs with only one HS (same conventions as in A). The ssODNs contain a 126 bp insert (green) coding for 3×Flag and GFP11 and HS targeting either the right or left side of the DSB (Table 2). FIG. 4C: normalized editing efficiency of ssODNs containing only one HS at the Lamin A/C and RAB11A loci. The polarity that allows pairing between the ssODN and resected ends (as shown in diagram in A) is favored. Sense and antisense ssODNs were tested in parallel experiments and their efficiency were normalized as follows: normalized efficiency of sense ssODN (light blue)=% GFP+ cells with sense ssODN/[% GFP+ cells with sense ssODN+% GFP+ cells with antisense ssODN]. Normalized efficiency of antisense ssODN (dark blue)=% GFP+ cells with antisense ssODN/[% GFP+ cells with sense ssODN+% GFP+ cells with antisense ssODN]. Numbers on top of each column indicate the non-normalized % of GFP+ cells for each ssODN determined by microscopy (Lamin A/C) or flow cytometer (RAB11A).

FIG. 5A-5B. Polarity of ssODNs affects incorporation of distal edits. FIG. 5A: schematics showing possible pairing interactions between resected locus (grey) and ssODNs (light or dark blue for sense and antisense ssODN respectively, arrows—3′ends) coding for a distal insert (green). Sequences between the DSB and insert were recoded to help integration of the distal insert and prevent cutting of edited locus by Cas9. FIG. 5B: normalized efficiency of sense vs antisense ssODNs calculated as in FIG. 4 (see Tables 3-4 for detailed results). Distance from the DSB, locus, and guide RNA polarity are indicated under each experiment. ssODN polarity has little effect on editing efficiency for proximal edits, but has a larger effect for distal edits. The favored polarity changes depending on whether the distal edit is positioned to the left or right of the DSB. Note that the favored ssODN polarity does not correlate with crRNA polarity (for example, first two columns in the graph show crRNAs 1776 and 1777 which cut at the same position but have opposite polarity). Experiments involving the PYM1 locus were done on HEK293T that were cloned out and genotyped by PCR genotyping (size shift) for 3×Flag insertion (see FIG. 6). All other experiments were performed on HEK233T (GFP1-10) cells that were directly scored for GFP+ by flow cytometer or microscopy 3 days after nucleofection. Numbers on top of each column indicate the overall % of edits. Note that overall frequency decreases with increasing distance from the DSB (also see FIG. 14).

FIG. 6A-6D. Recoding of sequences between the DSB and the edit increases recovery of distal edits. FIG. 6A: schematics showing resected locus (grey with arrow at the 3′ends, PYM1 locus) and ssODN donor (blue with arrow at the 3′end) coding for a proximal edit (green, restriction enzyme site, 1 bp to the right of the DSB) and a distal edit (red, 3× Flag, 23 bp to the left of the DSB). Double arrows represent the region between the proximal and distal edits that is recoded (silent mutations). FIG. 6B: graphs showing % of edited cells containing proximal+distal edits (purple), proximal only (green) or distal only (red), using a ssODN donor with or without a recoded region. >50 cell clones were analyzed by PCR genotyping (size shift) and RE digestion. FIG. 6C: schematics showing resected locus (grey with arrow at the 3′ends, Lamin A/C locus) and PCR donor (blue, thick bar) coding for a proximal edit (green, GFP11 inserted at the DSB) and a distal edit (red, tagRFP, 33 bp to the right of the DSB. Double arrows represent the region between proximal and distal edits that is recoded (silent mutations). FIG. 6D: graphs showing % of edited cells containing proximal+distal edits (purple), proximal only (green) or distal only (red), using a PCR donor with or without a recoded region. Edits were determined by direct examination of >1000 cells by microscopy.

FIG. 7A-7F. Repair is prone to template switching between donors. FIG. 7A: schematics showing repair of a DSB at the RAB11A locus (grey) with two ssODN donors. Arrows indicate 3′ ends. Donor 1 contains GFP11 (green) with a STOP codon (red cross) and two HS (blue). Donor 2 contains GFP11 with no STOP codon and no HS. Double arrows indicate identical sequence shared between the donors. FIG. 7B: graphs showing the percent of GFP+ cells (Y axis, as determined by flow cytometer) for each donor combination (X axis). Each bar represents the average insertion efficiency from two independent experiments (Table 4). Error bars represent the +/−SD. For comparison, an ssODN identical to donor 1 but without the STOP codon gives 17.2% edits (discontinuous right most bar). FIG. 7C: schematics showing repair of a DSB at the RAB11A locus as in diagram A but with two PCR donors (thick bars). FIG. 7D: graphs showing the percent of GFP+ cells as in graphs B but with two PCR donors. Each bar represents the average insertion efficiency from two independent experiments (Table 4). Error bars represent the +/−SD. FIG. 7E: schematics showing repair of a DSB at the Lamin A/C locus (grey) with two ssODN donors. Arrows represents 3′ends. Donor 1 contains GFP11 (green) and two HS (blue). Donor 2 contains a recoded GFP11 (stars) with no HS. Double arrows indicate identical sequence shared between the donors. In this experiment, the edits were amplified en masse by PCR using a locus-specific primer and an insert-specific primer and sequenced by Illumina sequencing. FIG. 7F: graph showing the % of reads with evidence of template switching (Y axis) for each donor combination (X axis). Donor 1+donor 2 without mutations and donor 1+donor 2 with 1 mutations every 3 nucleotides (1/3) show no evidence of template switching (0%), whereas donor 1+donor 2 (1/6) and donor 1+donor 2 (1/12) show evidence of template switching (0.5% and 1.4% respectively). See FIG. 15 and Table 15.

FIG. 8. Guidelines for donor design. FIG. 8A: schematic showing a typical editing experiment using a PCR fragment (thick line) with two homology arms (blue) to introduce an edit (green) at a distance from the DSB (stippled line). FIG. 8B: recommendations based on results presented in this study. For additional recommendations for ssODNs designed to insert edits at the DSB, see DeWitt et al., 121-11 METHODS 9-15 (2017) and Richardson et al., 34(3) NAT. BIOTECHNOL. 339-44 (2016).

FIG. 9. crRNAs used in this study. Schematics showing guide RNAs (arrows) used in this study mapped on Lamin A/C, RAB11A, SMC3, PYM1 (human) and Adcy3 (mouse) loci. Grey boxes indicate coding exons, only the first and last exons are shown for Lamin A/C, RAB11A, SMC3, and mouse Adcy3. For each guide, arrows indicate the 3′ end. Numbers indicate position of the DSB relative to the ATG or STOP codon. Chemically synthesized crRNAs were used at all loci, except for PYM1 where we used a plasmid-encoded sgRNA. Guide RNA sequences are in Table 14.

FIG. 10A-10C. Tagging with GFP of the SMC3 locus using PCR repair template with short homology arms. FIG. 10A: diagram showing PCR donor for GFP insertion at the SMC3 locus. Locus—grey, GFP—green, HS (Homology arm Sequences)—blue. GFP was inserted 5 bp to the right of the DSB. FIG. 10B: graphs showing % of GFP+ cells obtained with PCR fragments with HS of the indicated lengths. Insert size in all cases was 714 bp. Each bar represents the average insertion efficiency from two or more independent experiments (Table 1). Error bars represent the +/−SD. PCR fragments were nucleofected in HEK293T cells at the concentration indicated and cells were counted by flow cytometer 3 days later. FIG. 10C: confocal images of cells 3 days after nucleofection. GFP: green, DNA: blue. The GFP subcellular localization is as expected for in-frame translational fusion to SMC3, a nuclear protein.

FIG. 11A-11B. Flow cytometer plots of cells tagged with PCR repair templates. Flow cytometer plots showing the number of cells (Y axis) and their GFP intensity (X axis). FIG. 11A: Lamin A/C, RAB11A and SMC3 were targeted in HEK293T cells with an eGFP containing PCR fragment with or without ˜35 bp Homology arm Sequences (HS). Green double arrows indicate the % of GFP+ cells. For every experiment, non-nucleofected cells were also run through the flow cytometer to determine background fluorescence (<0.5% cells). Note that donors without HS yield GFP+ values slightly above background, consistent with a low level of integration by NHEJ or MMEJ. FIG. 11B: RAB11A was targeted in HEK293T (GFP1-10) cells using a GFP11-containing repair template with or without ˜35 bp Homology arm Sequences (HS). Green double arrows indicate the % of GFP+ cells. Non-nucleofected cells were also run through the flow cytometer to determine background fluorescence (<0.5% cells). Note that HEK293T cells that express GFP1-10 cells have a higher intrinsic fluorescence than HEK293T cells.

FIG. 12A-12B. Derivation of GFP+ and GFP− clones from a single editing experiment targeting the Lamin A/C locus with a GFP-containing PCR fragment. FIG. 12A: schematic showing the donor (green with blue Homology arm Sequences—HS) and targeted locus (grey). HEK293T cells were edited at the Lamin A/C locus with an eGFP PCR donor with 33/33 HS, and FACS-sorted as GFP+ and GFP− cells. The clones were amplified and examined by confocal microscopy. All GFP+ cells exhibit the expected nuclear membrane localization expected from a GFP translation fusion with Lamin A/C. FIG. 12B: statistics of genotyping results for GFP+ and GFP− single clones. See also FIG. 13.

FIG. 13A-13E. Structure of imprecise GFP knock-in edits. Schematics showing the GFP inserts obtained in the experiment described in FIG. 12. Lamin A/C locus (grey line), Full-length left HS (L, 33 bp) and right HS (R, 33 bp) (blue), GFP (green, with length of GFP sequence indicated), Indel (red). GFP+ indicates cells with Lamin A/C GFP signal. FIG. 13A: precise edit for reference. FIG. 13B: edits with imprecise right junctions—(b1) Contain an 11 bp duplication of the Lamin A/C locus sequence just downstream the right HS; (b2) Contain a 6 bp deletion of the Lamin A/C locus sequence just downstream the right HS; (b3) Contain a deletion of the last 19 bp of the right HS and of the 8 bp just downstream the right HS; (b4) Contain an 11 bp deletion inside the right HS; (b5) Contain only the 363 first bp of GFP sequence; (b6) Contain only the 70 first bp of GFP sequence followed by a 4 bp insertion and a full deletion of the right HS together with a 4 bp deletion of the Lamin A/C locus sequence just downstream the right HS sequence. Sequencing from wild-type size allele from Het GFP+ cell; and (b7) Contain only the 22 first bp of GFP sequence followed by a 5 bp insertion and a deletion of the first 13 bp of the right HS. Sequencing from wild-type size allele from Het GFP+ cell. FIG. 13C: edits with imprecise left junctions—(c1) Contain a 23 bp duplication of the left HS just upstream the GFP sequence; (c2) Contain on the left side the 8 first bp of GFP, followed by the 25 bp of the left HS sequence upstream of GFP, and followed by full-length GFP sequence; (c3) Contain a 52 bp insertion followed by the last 469 bp of GFP sequence; and (c4) Contain a deletion of the last 7 bp of the left HS followed by the last 68 bp of GFP sequence. Sequencing from wild-type size allele from Het GFP+ cell. FIG. 13D: edit with internal deletion—(d1) Contain the 556 first bp of GFP sequence followed by a 12 bp insertion and the last 13 bp of GFP sequence. FIG. 13E: eEdit with inverted insertion—(e1) Contain the left HS and first 501 bp of GFP sequence inverted.

FIG. 14. Insertion efficiency relative to distance from the DSB. Graph showing the efficiency % of editing (Y axis) vs distance from the DSB (X axis) for PCR donors (RAB11A, cr1777, GFP11 insertion) or ssODNs (GFP11 insertion (see FIG. 5) except for PYM1 were 3×Flag insertion was monitored by genotyping single cell colonies (see FIG. 6)). Each line links editing experiments performed with the same guide RNA. ssODNs (optimal polarity, FIG. 5) were designed to insert the edit at varying distances from the DSB as indicated. The sequence between the edit and the DSB was partially recoded to improve insertion efficiency (FIG. 6) and Cas9 re-cutting of the edited locus while preserving coding potential.

FIG. 15A-15B. Illumina sequencing to monitor template switching. FIG. 15A: schematic representation of the experimental design (see FIG. 7). Stars in color represent silent mutations (A, C, G or T) used to monitor template switching. FIG. 15B: the probability of a mutation (relative to the “No mutation” template) at each nucleotide position in the region of the ssODN repair template, after removal of incompletely mapped and low-quality reads. Bars are color-coded by identity of the incorporated nucleotide. Green: A, blue: C, black: G, red: T. PCR control: Two cell populations that received separately a wild-type ssODN or a mutant ssODN (1/6 mutations) were combined for PCR amplification. This control was used to determine basal levels of template switching that might occur during PCR amplification. These levels are 25-fold lower than observed in cells co-transfected with the wild-type ssODN (donor 1) and a 1/6 mutations ssODN (donor 2) (0.02% versus 0.50%).

FIG. 16A-16E. Schematics showing repair of a DSB (grey) using two ssODN donors. Donor 1 contains an insert (green) and two homology arms (blue). Donor 2 contains the same insert with mutations (red stars) and no homology arms. Arrows indicate 3′ ends and dotted lines represented newly synthesized DNA. FIG. 16A: strand invasion—the DSB is resected creating two 3′ overhangs on each side of the DSB. The right overhang pairs with Donor 1 and is extended by DNA synthesis. FIGS. 16B and 16C: template switching—the newly synthetized strand withdraws from Donor 1 and anneals to Donor 2 (B), and withdraws from Donor 2 and anneals back to Donor 1 (C). FIG. 16D: annealing—the newly synthesized strand withdraws from Donor 1 and anneals back to the locus. FIG. 16E: second strand synthesis and ligation—the newly synthesized strand is used as a template for second strand synthesis. The resulting edit is a hybrid insertion containing sequences from Donor 1 and Donor 2.

FIG. 17A-17B. Comparison between nucleofection (FIG. 17A) and Lipofection (FIG. 17B) in HEK293T cells.

FIG. 18A-18C. Various gene tagging in HEK293T cells using PCR or gBlock donors. FIG. 18A—Nucleofection. FIG. 18B—Lipofection. FIG. 18C—Expression patterns.

FIG. 19A-19B. GFP/RFP co-taggin in HEK293T cells. FIG. 19A—two genes editing (nucleofection). FIG. 19B—two colors editing (lipofection).

FIG. 20. Isolation of HEK293T cells with stress granules proteins tagged with GFP.

FIG. 21A-21B. U2OS and DLD1 genes tagging. FIG. 21A—U2OS (nucleotfection). FIG. 21B—DLD1 (nucleofection).

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

Unless otherwise indicated, the terms “polynucleotide” and “nucleic acid” refer to a deoxyribonucleotide or ribonucleotide polymer, in linear or circular conformation, and in either single- or double-stranded form. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, an “edit” is the desired modification to be introduced into the genome. In other words, an edit is any change in the genomic sequence that is included in the repair template polynucleotide. Edits can include, for example, base pair insertions, deletions or changes.

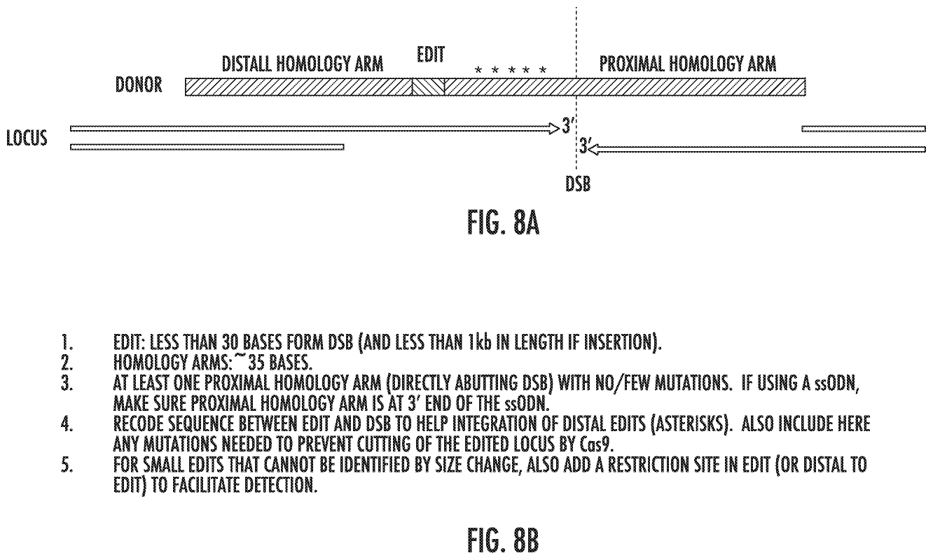

The term “intervening sequence” refers to a sequence between the edit and the double-stranded break (DSB). An intervening sequence can be unmodified (identical to genome sequence) or can be modified (for example, see FIG. 8.).

As used herein, a “homology arm,” “homology sequence” or “sequence homologous” to a reference or target gene/sequence describes a polynucleotide sequence that has substantial sequence identity to a corresponding segment of the reference or target gene/sequence, e.g., at least 75, 80, 85, 90, 91, 92, 93, 94, 95, 96, 97, 98, 99% identical or even 100% identical, to the nucleotide sequence of the reference or target gene/sequence, such that, when placed under appropriate conditions, homologous recombination can take place between a pair of “homologous sequences” and their reference or target gene/sequence. The homology arms have substantial sequence identity to the sequence upstream and downstream of the targeted site in the target nucleic acid molecule.

For edits inserted to the right of a DSB: the right homology arm corresponds to the genomic sequence immediately to the right of the insertion point of the edit and the left homology arm corresponds to the genomic sequence immediately on the left side of the DSB.

For edits inserted to the left of a DSB: the left homology arm corresponds to the genomic sequence immediately to the left of the insertion point of the edit and the right homology arm corresponds to the genomic sequence immediately on the right side of the DSB.

The terms “target sequence,” “target nucleic acid” or “target DNA sequence,” when used to refer to a pre-determined segment of a genomic sequence or polynucleotide is similarly defined in regard to the percentage sequence identity between the target sequence and its corresponding guide RNA. On the other hand, a “homology arm” or “target sequence” is of the appropriate length that ensures its purpose. Typically, a “homology arm” is in the size range of about 10-100, 10-90, 10-80, 15-75, 15-70, 15-65, 15-60, 15-55, 15-50, 15-45, 15-40, 15-35, 20-50, 20-45, 20-40, 20-35, 25-40, 25-35 or 30-35 nucleotides (e.g., about 30, 35, 40, 45, 50, 55 or 60 nucleotides in length); whereas a “target sequence” may vary in the size range of about 10-50, 15-45, or 20-40 (e.g., about 20, 25, or 30) nucleotides. In some embodiments, the target sequence contains a sequence that is suitable as a substrate for an RNA-guided DNA endonuclease (e.g., a Cas9 nuclease) (i.e., a nuclease target sequence site). In some embodiments, the target sequence contains a sequence that is suitable as a substrate for Cfp1 endonuclease (i.e., an endonuclease target sequence site).

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, 2 ADVANCES IN APPLIED MATHEMATICS 482-89 (1981). This algorithm can be applied to amino acid sequences by using the scoring matrix developed by Dayhoff, 3 (5 Suppl.) ATLAS OF PROTEIN SEQUENCES AND STRUCTURE, M. O. Dayhoff ed. 353-58, National Biomedical Research Foundation, Washington, D.C., USA, and normalized by Gribskov et al., 14(6) NUCL. ACIDS RES. 6745-63 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

“Cas9” or (CRISPR associated protein 9) is an RNA-guided DNA endonuclease enzyme associated with the CRISPR (Clustered Regularly Interspersed Palindromic Repeats) adaptive immunity system in Streptococcus pyogenes, among other bacteria. S. pyogenes utilizes the CRISPR system to memorize and later interrogate and cleave foreign DNA, such as the DNA of an invading bacteriophage. Cas9, complexed with a guide RNA, performs this interrogation by unwinding foreign DNA and checking whether the DNA contains any sequence segment complementary to a spacer region of the guide RNA. If the guide RNA finds sequence complementarity in the DNA, it is cleaved by Cas9.

“Cpf1” or “CRISPR/Cpf1” is a DNA editing technology analogous to the CRISPR/Cas9 system. Cpf1 is an RNA-guided DNA endonuclease enzyme associated with the CRISPR adaptive immunity system in Prevotella and Francisella, among other bacteria. Cpf1 is a smaller and simpler endonuclease as compared to Cas9 because Cpf1 only requires one RNA molecule to cut DNA while Cas9 requires two. Cpf1 is a Type V CRISPR/Cas system containing a 1,300 amino acid protein.

As used herein, “sgRNA” or “small guide RNA” refers to a short RNA molecule that is capable of forming a complex with Cas9 protein and contains a segment of about 20 nucleotides complementary to a target DNA sequence, such that the Cas9-sgRNA complex directs Cas9 cleavage of a target DNA sequence upon the sgRNA recognizing the complementary sequence in the target DNA sequence. Accordingly, a sgRNA is approximately a 20-base sequence (ranging from about 10-50, 15-45, or 20-40, for example, 15, 20, 25, or 30 bases) specific to the target DNA 5′ of a non-variable scaffold sequence.

As used herein, the term “endogenous sequence” refers to a chromosomal sequence that is native to the cell.

The term “exogenous,” as used herein, refers to a sequence that is not native to the cell, or a chromosomal sequence whose native location in the genome of the cell is in a different chromosomal location.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

### II. RNA-Guided Endonucleases

In particular embodiments, the compositions and methods of the present invention utilize RNA-guided endonucleases. In some embodiments, the endonuclease comprises at least one nuclear localization signal, which permits entry of the endonuclease into the nuclei of eukaryotic cells and embryos such as, for example, non-human one-cell embryos. In other embodiments, RNA-guided endonucleases comprise at least one nuclease domain and at least one domain that interacts with a guide RNA. An RNA-guided endonuclease is directed to a specific nucleic acid sequence (or target sequence/site) by a guide RNA. The guide RNA interacts with the RNA-guided endonuclease as well as the target site such that, once directed to the target site, the RNA-guided endonuclease is able to introduce a double-stranded break into the target site nucleic acid sequence. Since the guide RNA provides the specificity for the targeted cleavage, the endonuclease of the RNA-guided endonuclease is universal and can be used with different guide RNAs to cleave different target nucleic acid sequences. The RNA-guided endonuclease can be derived from a clustered regularly interspersed short palindromic repeats (CRISPR)/CRISPR-associated (Cas) system. The CRISPR/Cas system can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In one embodiment, the RNA-guided endonuclease is derived from a type II CRISPR/Cas system. In specific embodiments, the RNA-guided endonuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum the mopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In other embodiments, the RNA-guided endonuclease is derived from another Cas nuclease including, but not limited to, Cpf1, C2c1, C2c2, and C2c3 proteins. Cpf1 is similar to Cas9, and contains a RuvC-like nuclease domain. See Zetsche et al., 163 CELL 1-13 (2015).

### III. Guide RNA

In some embodiments of the present disclosure, a CRISPR/Cas nuclease system includes at least one guide RNA. In some embodiments, the guide RNA and the Cas protein may form a ribonucleoprotein (RNP), e.g., a CRISPR/Cas complex. The guide RNA may guide the Cas protein to a target sequence on a target nucleic acid molecule, where the guide RNA hybridizes with, and the Cas protein cleaves, the target sequence. In some embodiments, the CRISPR/Cas complex may be a Cpf1/guide RNA complex. In some embodiments, the CRISPR complex may be a Type-II CRISPR/Cas9 complex. In some embodiments, the Cas protein may be a Cas9 protein. In some embodiments, the CRISPR/Cas9 complex may be a Cas9/guide RNA complex.

A guide RNA for a CRISPR/Cas9 nuclease system comprises a CRISPR RNA (crRNA) and a tracr RNA (tracr). In another embodiment, a single guide RNA (sgRNA)—a chimer of cr/tracrRNA—can be used. See Doudna, J. A. & Charpentier, E., 346(6213) SCIENCE 1258096 (2014). A guide RNA for a CRISPR/Cpf1 nuclease system comprises a crRNA. In some embodiments, the crRNA may comprise a targeting sequence that is complementary to and hybridizes with the target sequence on the target nucleic acid molecule. The crRNA may also comprise a flagpole that is complementary to and hybridizes with a portion of the tracrRNA. In some embodiments, the crRNA may parallel the structure of a naturally occurring crRNA transcribed from a CRISPR locus of a bacteria, where the targeting sequence acts as the spacer of the CRISPR/Cas9 system, and the flagpole corresponds to a portion of a repeat sequence flanking the spacers on the CRISPR locus.

The guide RNA may target any sequence of interest via the targeting sequence of the crRNA. In some embodiments, the degree of complementarity between the targeting sequence of the guide RNA and the target sequence on the target nucleic acid molecule may be about 70%, 75%, 80%, 85%, 90%, 95%, 97%, 98%, 99%, or 100%. In some embodiments, the targeting sequence of the guide RNA and the target sequence on the target nucleic acid molecule may be 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches. In some embodiments, the targeting sequence of the guide RNA and the target sequence on the target nucleic acid molecule may contain 5 or 6 mismatches.

The length of the targeting sequence of the guide RNA may depend on the CRISPR/Cas9 system and components used. For example, different Cas9 proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence may comprise 18-24 nucleotides in length. In some embodiments, the targeting sequence may comprise 19-21 nucleotides in length. In some embodiments, the targeting sequence may comprise 20 nucleotides in length.

### IV. Target Site/Sequence of the Target Nucleic Acid Molecule

An RNA-guided endonuclease in conjunction with a guide RNA is directed to a target site in the chromosomal sequence, wherein the RNA-guided endonuclease introduces a double-stranded break in the chromosomal sequence. The target site has no sequence limitation except that the sequence is immediately followed (downstream) by a consensus sequence. This consensus sequence is also known as a protospacer adjacent motif (PAM). Examples of PAMs include, but are not limited to, NGG, NGGNG, and NNAGAAW (wherein N is defined as any nucleotide and W is defined as either A or T). In particular embodiments, the first region (at the 5′ end) of the guide RNA is complementary to the protospacer of the target sequence. Typically, the first region of the guide RNA is about 19 to 21 nucleotides in length. Thus, in certain aspects, the sequence of the target site in the chromosomal sequence is 5′-N₁₉₋₂₁-NGG-3′. The PAM is in italics.

The target site can be in the coding region of a gene, in an intron of a gene, in a control region of a gene, in a non-coding region between genes, etc. The gene can be a protein coding gene or an RNA coding gene. The gene can be any gene of interest.

### V. Linear Donor Polynucleotides & Design Parameters Thereof

In certain embodiments, the present invention provides a double-stranded, linear donor polynucleotide comprising a template polynucleotide encoding an edit flanked by an intervening sequence and two homology arms. In other embodiments, the donor polynucleotide comprises a template polynucleotide encoding an edit flanked by two homology arms.

In some embodiments, the template polynucleotide of the double-stranded, linear donor polynucleotide may correspond to an endogenous sequence of a target cell. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell. In some embodiments, the template sequence may be substantially identical to a portion of the endogenous sequence in a cell at or near the cleavage site, but comprise at least one nucleotide change (i.e., an “edit” as defined herein). In some embodiments, the repair of the cleaved target nucleic acid molecule with the template may result in an edit comprising an insertion, deletion, or substitution of one or more nucleotides of the target nucleic acid molecule. In some embodiments, the edit may result in one or more amino acid changes in a protein expressed from a gene comprising the target sequence. In some embodiments, the edit or mutation may result in one or more nucleotide changes in an RNA expressed from the target gene. In some embodiments, the edit may alter the expression level of the target gene. In some embodiments, the edit may result in increased or decreased expression of the target gene. In some embodiments, the edit may result in gene knockdown. In some embodiments, the edit may result in gene knockout. In some embodiments, the repair of the cleaved target nucleic acid molecule with the template may result in replacement of an exon sequence, an intron sequence, a transcriptional control sequence, a translational control sequence, or a non-coding sequence of the target gene.

In other embodiments, the double-stranded, linear donor polynucleotide encoding an edit may comprise an exogenous sequence. In some embodiments, the exogenous sequence may comprise a protein or RNA coding sequence operably linked to an exogenous promoter sequence such that, upon integration of the exogenous sequence into the target nucleic acid molecule, the cell is capable of expressing the protein or RNA encoded by the integrated sequence. In other embodiments, upon integration of the exogenous sequence into the target nucleic acid molecule, the expression of the integrated sequence may be regulated by an endogenous promoter sequence. In some embodiments, the exogenous sequence may be a chromosomal or extrachromosomal sequence. In some embodiments, the exogenous sequence may provide a cDNA sequence encoding a protein or a portion of the protein. In yet other embodiments, the exogenous sequence may comprise an exon sequence, an intron sequence, a transcriptional control sequence, a translational control sequence, or a non-coding sequence. In some embodiments, the integration of the exogenous sequence may result in gene knock-in.

In the double-stranded, linear donor polynucleotide, the template polynucleotide is flanked by a first homology arm and a second homology arm, e.g., a left homology arm and a right homology arm. These sequences to the left and right of the template polynucleotide have substantial sequence identity to sequences located to the left and right, respectively, of the target site of the RNA-guided endonuclease in the target nucleic acid molecule. Because of these sequence similarities, homology arms permit homologous recombination between the donor polynucleotide and the targeted sequence such that the template polynucleotide can serve as a template for DNA synthesis. In certain embodiments, the linear donor polynucleotide comprises a template polynucleotide encoding an edit flanked by an intervening sequence and two homology arms.

In certain embodiments, specifically, for edits inserted to the right of a DSB, the right homology arm corresponds to the genomic sequence immediately to the right of the insertion point of the edit and the left homology arm corresponds to the genomic sequence immediately on the left side of the DSB. In other embodiments, specifically, for edits inserted to the left of a DSB, the left homology arm corresponds to the genomic sequence immediately to the left of the insertion point of the edit and the right homology arm corresponds to the genomic sequence immediately on the right side of the DSB.

In particular embodiments, each homology arm can range in length from about 10 nucleotides to about 100 nucleotides. The recited range includes ranges within the recited range including, but not limited to, 10-100, 10-90, 10-80, 15-75, 15-70, 15-65, 15-60, 15-55, 15-50, 15-45, 15-40, 15-35, 20-50, 20-45, 20-40, 20-35, 25-40, 25-35 or 30-35 nucleotides (e.g., about 30, 35, 40, 45, 50, 55 or 60 nucleotides in length). In a specific embodiment, a homology arm is 15-60 nucleotides in length. In another embodiment, a homology arm is 25-45 nucleotides in length. In yet another embodiment, a homology arm is 30-40 nucleotides in length. In a further embodiment, a homology arm is 35 nucleotides in length. In certain embodiments, homology arms can comprise different lengths within the range.

### VI. Introducing Genome Editing Compositions into the Cell or Embryo

The RNA-guided endonuclease(s) (or encoding nucleic acid), the guide RNA(s) (or encoding DNA), and the double-stranded, linear donor polynucleotide can be introduced into a cell or embryo by a variety of means. In some embodiments, the cell or embryo is transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art. In other embodiments, the molecules are introduced into the cell or embryo by microinjection. In certain embodiments, the embryo is a fertilized one-cell stage embryo of the species of interest. In such embodiments, the molecules can be injected into the pronuclei of one-cell embryos.

The RNA-guided endonuclease(s) (or encoding nucleic acid), the guide RNA(s) (or DNAs encoding the guide RNA), and the double-stranded, linear donor polynucleotide(s) can be introduced into the cell or embryo simultaneously or sequentially. The ratio of the RNA-guided endonuclease(s) (or encoding nucleic acid) to the guide RNA(s) (or encoding DNA) generally will be about stoichiometric such that they can form an RNA-protein complex. In one embodiment, DNA encoding an RNA-guided endonuclease and DNA encoding a guide RNA are delivered together within a plasmid vector.

In further embodiments, the method comprises maintaining the cell or embryo under appropriate conditions such that the guide RNA(s) directs the RNA-guided endonuclease(s) to the targeted site(s) in the chromosomal sequence, and the RNA-guided endonuclease(s) introduce at least one double-stranded break in the chromosomal sequence. A double-stranded break can be repaired by a DNA repair process such that the chromosomal sequence is modified by a deletion of at least one nucleotide, an insertion of at least one nucleotide, a substitution of at least one nucleotide, or a combination thereof.

In general, the cell is maintained under conditions appropriate for cell growth and/or maintenance. Suitable cell culture conditions are well known in the art. Those of skill in the art appreciate that methods for culturing cells can and will vary depending on the cell type. Routine optimization may be used, in all cases, to determine the best techniques for a particular cell type.

An embryo can be cultured in vitro (e.g., in cell culture). Typically, the embryo is cultured at an appropriate temperature and in appropriate media with the necessary O₂/CO₂ ratio to allow the expression of the RNA endonuclease and guide RNA, if necessary. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. In certain embodiments, the female host is pseudo-pregnant. Methods of preparing pseudo-pregnant female hosts are known in the art. Additionally, methods of transferring an embryo into a female host are known. Culturing an embryo in vivo permits the embryo to develop and can result in a live birth of an animal derived from the embryo. Such an animal would comprise the modified chromosomal sequence in every cell of the body.

### VII. Cell and Embryo Types

A variety of eukaryotic cells and embryos are suitable for use in the method. For example, the cell can be a human cell, a non-human mammalian cell, a non-mammalian vertebrate cell, an invertebrate cell, an insect cell, a plant cell, a yeast cell, or a single cell eukaryotic organism. In general, the embryo is non-human mammalian embryo. In specific embodiments, the embryos can be a one-cell non-human mammalian embryo. Exemplary mammalian embryos, including one-cell embryos, include without limit mouse, rat, hamster, rodent, rabbit, feline, canine, ovine, porcine, bovine, equine, and primate embryos. In still other embodiments, the cell can be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others. In exemplary embodiments, the cell is a mammalian cell.

Non-limiting examples of suitable mammalian cells include Chinese hamster ovary (CHO) cells, baby hamster kidney (BHK) cells; mouse myeloma NSO cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Hepalclc7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells. An extensive list of mammalian cell lines may be found in the American Type Culture Collection catalog (ATCC, Manassas, Va.).

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Precision Genome Editing Using Synthesis-Dependent Repair of Cas9-Induced DNA Breaks

The RNA-guided DNA endonuclease Cas9 has emerged as a powerful new tool for genome engineering. Cas9 creates targeted double-strand breaks (DSBs) in the genome. Knock-in of specific mutations (precision genome editing) requires homology-directed repair (HDR) of the DSB by synthetic donor DNAs containing the desired edits, but HDR has been reported to be variably efficient. Here, we report that linear DNAs (single and double-stranded) engage in a high-efficiency HDR mechanism that requires only about 35 nucleotides of homology with the targeted locus to introduce edits ranging from about 1 to 1000 nucleotides. We demonstrate the utility of linear donors by introducing fluorescent protein tags in human cells and mouse embryos using PCR fragments. We find that repair is local, polarity-sensitive, and prone to template switching, characteristics that are consistent with gene conversion by synthesis-dependent strand-annealing (SDSA). Our findings enable rational design of synthetic donor DNAs for efficient genome editing.

We documented previously that, in C. elegans, HDR can be very efficient provided that the donor DNAs are linear (Paix et al., 44(15) NUCLEIC ACIDS RES. e128 (2016)). Linear donors do not appear to integrate at the DSB, but instead are used as templates for DNA synthesis, as in the synthesis-dependent strand annealing (SDSA) model for gene conversion (Mehta et al., 65(3) MOL. CELL 515-26 e513 (2017); Jasin et al. (2016); Paques, F. & Haber, J. E., 63(2) MICROBIOL. MOL. BIOL. REV. 349-404 (1999)). In C. elegans, donors for SDSA can be single (ssODNs) or double-stranded (PCR fragments), and require only short homology arms (˜35 bases) to engage the DSB. The repair process is sensitive to insert size and prone to template switching, where synthesis can “jump” between two overlapping donors (Paix et al. (2016)). In human cells, SDSA has been proposed as a repair mechanism for ssODNs (Kan et al., 27(7) GENOME RES. 1099-1111 (2017); Liang et al. (2016)), but not for double-stranded donors, which are thought to participate in a different HDR pathway (Kan et al., 10(4) PLOS GENET. E1004251 (2014)) (Bothmer et al., 8 NAT. COMMUN. 13905 (2017)). Here, we investigate how linear donors engage the DSB repair machinery in mammalian cells. First, we demonstrate that, as in C. elegans, PCR fragments with 35 bp homology arms function as efficient donors for genome editing in mouse embryos and human cells. Using PCR fragments and ssODNs, we investigate the sequence requirements for efficient repair by linear donors in human cells. Our findings are consistent with SDSA and suggest simple donor DNA design principles to maximize editing efficiency.

### Materials and Methods

Detailed Results, Sequences and Solutions.

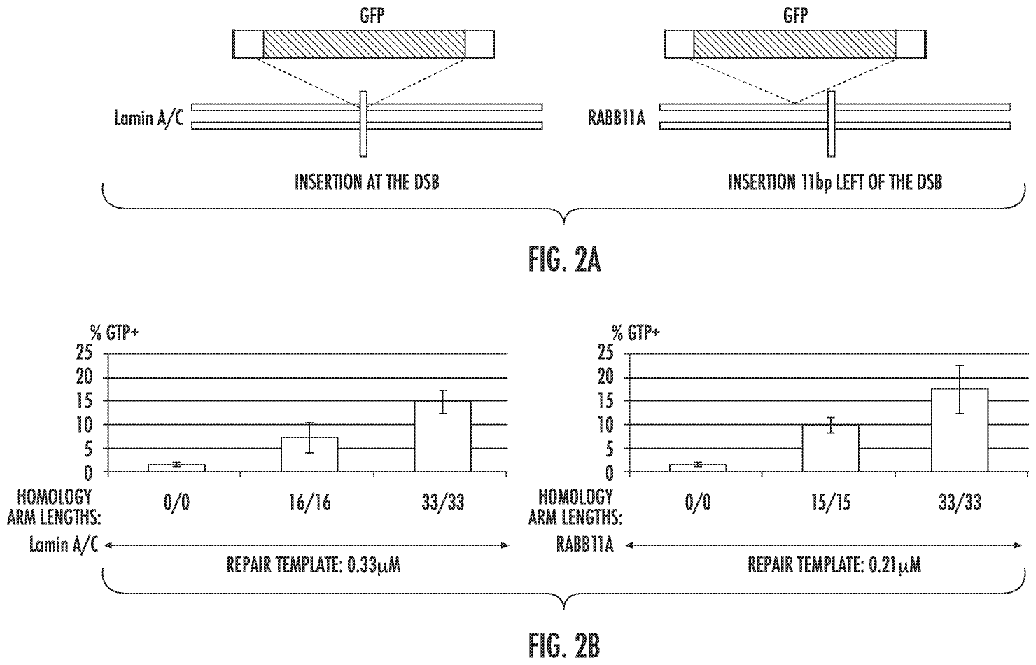

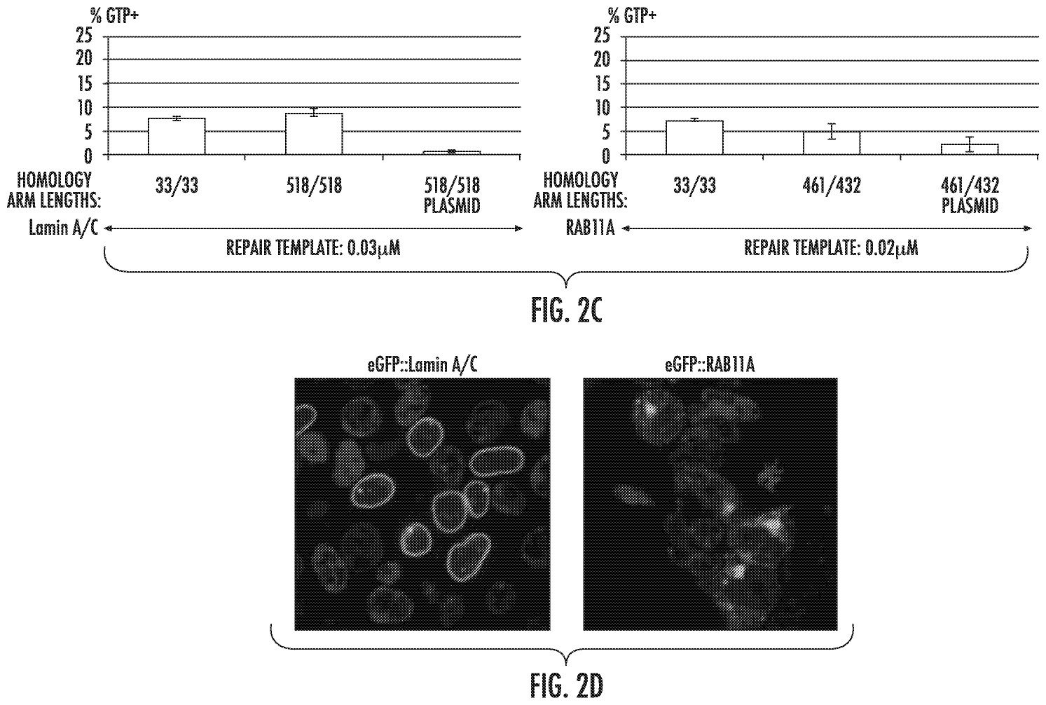

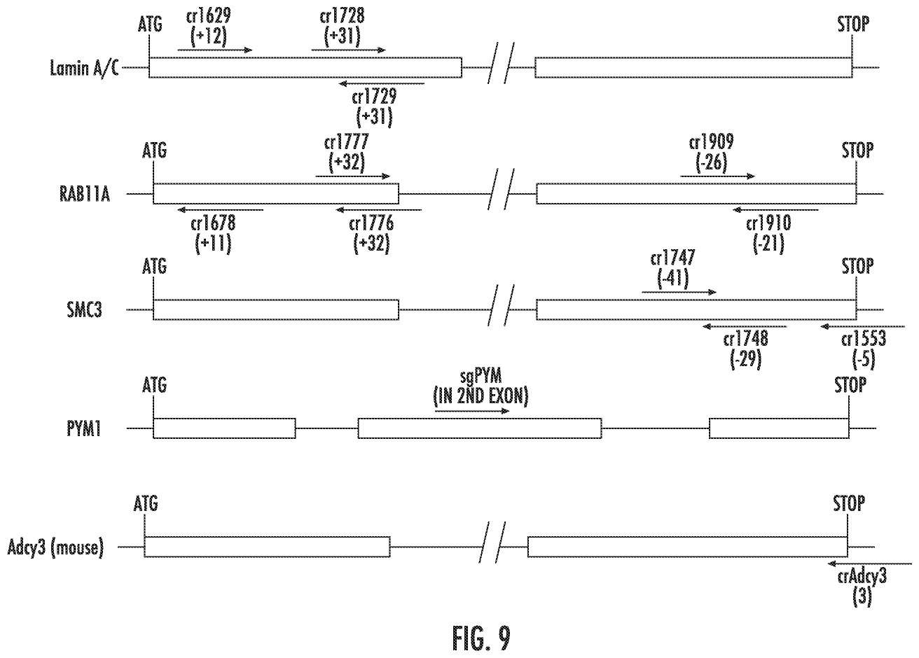

Tables 1-3 lists all experiments, including detailed conditions and results of experimental replicates. Table 5-14 lists sequences of linear donors, plasmids, PCR primers and cr/sgRNAs, respectively. Position of the cr/sgRNAs on the loci targeted in this study can be found in FIG. 9. Results presented in FIGS. 2, 3, 7B/D and 10 are the average of at least two independent experiments and the error bars represent the standard deviation (SD).

Repair Templates, Cas9, Cr/tracrRNAs and Plasmids for Cell Culture.

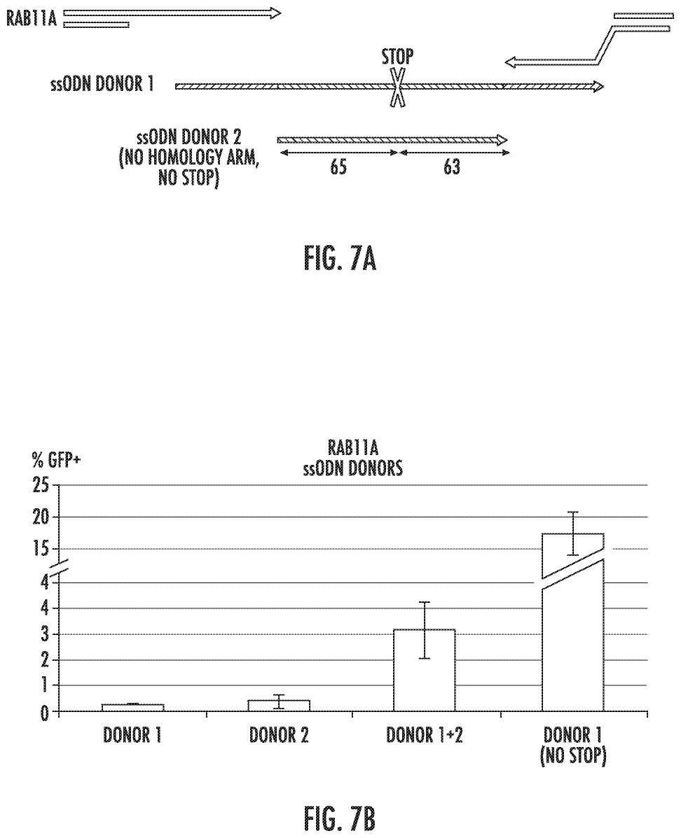

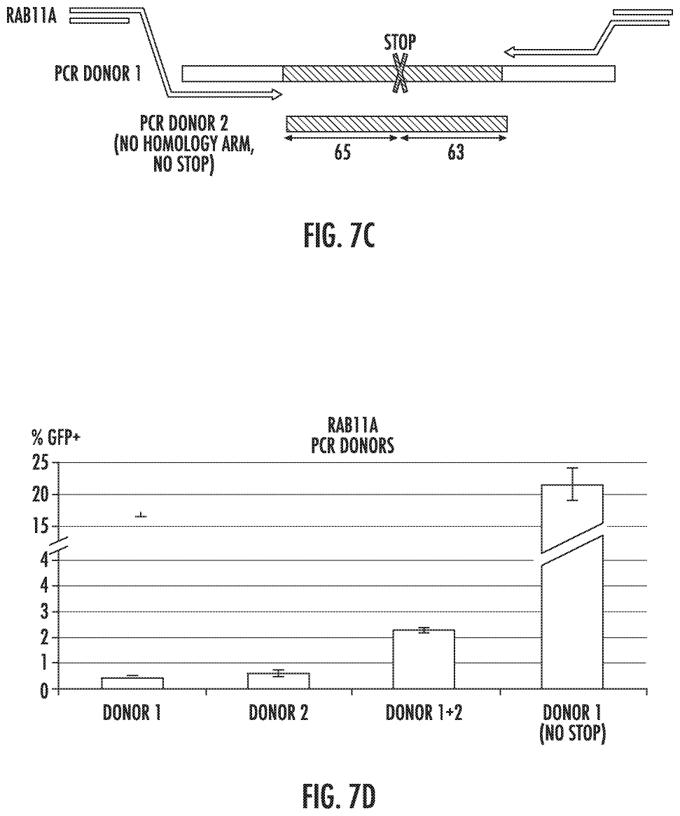

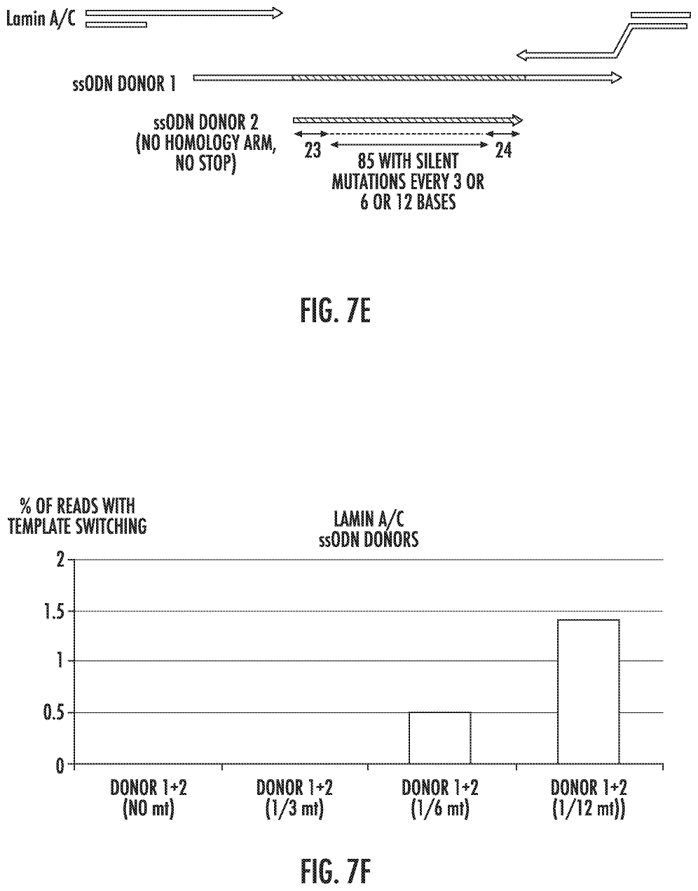

ssODNs (ultramers) and PCR primers where ordered from IDT and reconstituted at 50 μM and 100 μM respectively in water. For the Illumina sequencing experiment shown in FIG. 7F, ssODNs and primers were ordered PAGE purified. PCR fragment donors were synthesized as described in Paix et al., 121-122 METHODS 86-93 (2017).

Cas9 protein was purified as described in Paix et al., 201(1) GENETICS 47-54 (2015). crRNAs and tracrRNA were ordered from IDT and reconstituted in 5 mM Tris-HCl pH7.5 at 130 μM. Plasmids containing repair templates were made using gBlock gene fragments (IDT) and InFusion cloning kit (Clontech), and purified using Qiagen mini-prep kit and eluted in H2O. For experiments at the PYM1 locus, the sgRNA was cloned as described in Moyer et al., 129 METHODS CELL BIOL. 19-36 (2015).

Cas9 RNP Nucleofection.

With the exception of experiments at the PYM1 locus (see below), all experiments in this study used Cas9 RNP delivery (DeWitt et al., 121-122 METHODS 9-15 (2017)). Nucleofections using Cas9 RNP were performed as described (Leonetti et al., 113(25) PROC. NATL. ACAD. SCI. USA E3501-08 (2016)). HEK293T cells or HEK293T cells expressing a truncated GFP (GFP1-10) (Kamiyama et al., 7 NAT. COMMUN. 11046 (2016)) were grown to 50-75% confluency, trypsinized, pelleted and resuspended at 800000 cells/80 μl of PBS. Just before nucleofection, PBS was replaced with 80 μl of Nucleofection kit V (Lonza). 40 μl of Cas9 RNP mix (see below) was added to the cells in suspension in Nucleofector kit V and processed using an Amaxa Nucleofector 2b machine (Lonza) using the A023 program. Cells were transferred to culture media and analyzed for fluorescence 3 days after.

The Cas9 RNP mix contains: 6.5 μM of crRNA and tracrRNA, 9.8 μM of Cas9 (1.6 μg/μl), a variable concentration of repair templates (see Tables 1-3 for details), 10.4% Glycerol, 131 mM KCl, 5.2 mM Hepes, 1 mM MgCl2, 0.5 mM Tris-HCl, pH7.5.

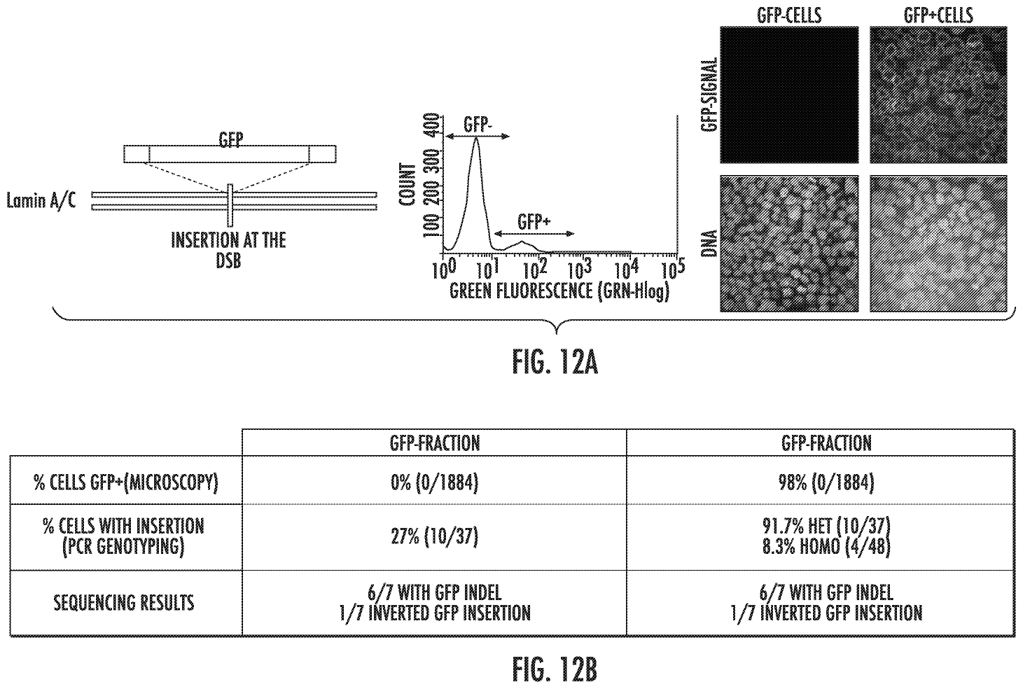

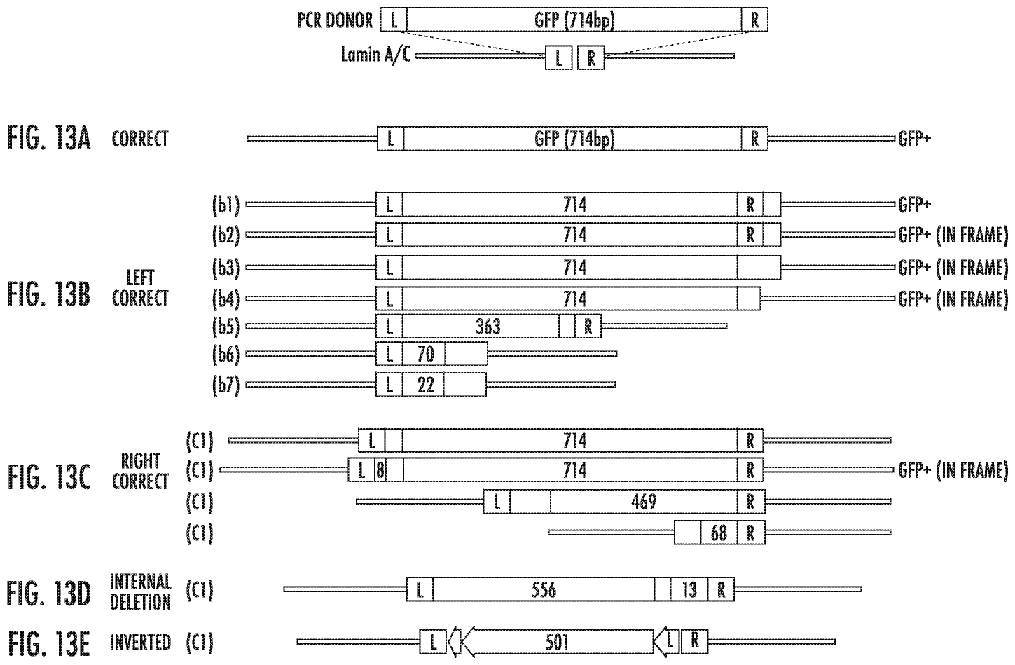

For sequencing of GFP edits at the Lamin A/C locus, cells were sorted (at the JHU Ross Flow Cytometry Core Facility) for GFP signal and cloned in 96 wells plates for genotyping or pooled in a 6-well plate for microscopy analysis. Single cell clones were lysed using QuickExtract DNA Extraction Solution (Epicentre) and genotyped by PCR using Phusion taq (NEB) with genomic primers outside of the HDR fragment. PCR products were analyzed on agarose gel and sequenced (see FIGS. 12 and 13).

Cas9 Plasmid Transfections.

For experiments at the PYM1 locus, Cas9 and the sgRNA were delivered on plasmids. HEK293T cells were grown to 50-75% confluency in 6 wells plate (with 2 ml of culture media per wells). 10.8 μl of Cas9 plasmid mix (containing 3.6 μl of X-tremeGENE 9 DNA Transfection Reagent from Roche, 892 ng of plasmid pX458 containing PYM1 sgRNA and 3.24 pmol of repair template) was added to 120l of optiMEM glutaMAX media (ThermoFisher), incubated for 15 min at room temperature, and next added to the cells. 48 h after transfection, cells were sorted for GFP signal (to select for cells that received pX458) and grown out as single cell clones. The single cell clones were lysed and genotyped by PCR. PCR products were directly analyzed on agarose gel or mix with EcoR1 (NEB) and the corresponding Restriction Enzyme (RE) buffer, digested over-night and analyzed on agarose gel.

Cytometer Analysis.

For each experiment, 5000 to 10000 cells were analyzed using a Guava EasyCyte 6/2L (Millipore) cytometer. Cells were scored as GFP+ if they exhibited a higher signal than 99.5% of non-transfected control cells. HEK293T (GFP1-10) cells exhibit a higher basal green fluorescence than wild-type HEK293T cells. Cytometer analysis could not be performed on these cells for GFP11-tagged Lamin A/C and SMC3. For those experiments, as well as for RFP tagging, cells were analyzed by fluorescence microscopy and scored manually.

Microscopy.

Cells were fixed in 4% PFA and mounted with DAPI. Cells were imaged using a confocal microscope with a 63× objective. >50 fields of cells (>1000 cells) were selected in the DAPI channel, photographed, and analyzed for GFP or RFP expression manually.

PCR Amplicons for Illumina Sequencing.

HEK293T (GFP1-10) were nucleofected with different combinations of repair ssODNs (FIG. 7E, Tables 1-3). To control for possible template-switching during PCR amplification, we also introduced single donors (wild-type or mutant) in two separate cell populations and combined the cells during PCR amplification. 60 h after nucleofection, cells were trypsinized, washed in PBS, and 500000 cells were lysed in 40 μl of QuickExtract DNA Extraction Solution. 40 μl of H2O was added to each lysis. A total of 6 μl of DNA from each experiments were PCR amplified using Phusion Taq and the primer 390 (Forward, in the left end of the insert) and the primer 1849 (Reverse, in the Lamin A/C locus downstream of the right HS of the ssODN used for repair) for 10 cycles at 68.5° C. (see Tables 10-13 for primer sequences). After 10 PCR cycles, no band could be detected on agarose gel and ethidium bromide staining. Each PCR reaction was purified using Qiagen Minelute columns and eluted in 10 μl of H2O. 2 μl of each PCR were amplified using Phusion taq at 65° C. for 20 cycles. PCR reactions did not reach an amplification plateau with this number of cycles. The PCR reactions were performed using primers 1928 (Forward, containing the Illumina sequence and annealing in the same region than primer 390) and Reverse primers containing the Illumina sequence and a specific barcode. The Illumina reverse primers anneal with the Lamin A/C locus just upstream of primer 1849 and downstream the right HS of the ssODN used for repair.

PCR amplicons were purified on a 10% non-denaturing TBE/PAGE gel and the band corresponding to the PCR product was cut from the gel, eluted over-night, and precipitated with isopropanol. After resuspension, sample concentrations were quantified on a bioanalyzer, and the barcoded samples were pooled to a concentration of 0.4 μM per sample in 10p. This sample was submitted to the Johns Hopkins School of Medicine Genetics Resources Core Facility for 250 cycle paired-end sequencing on an Illumina MiSeq instrument.

Illumina Sequencing Analysis.

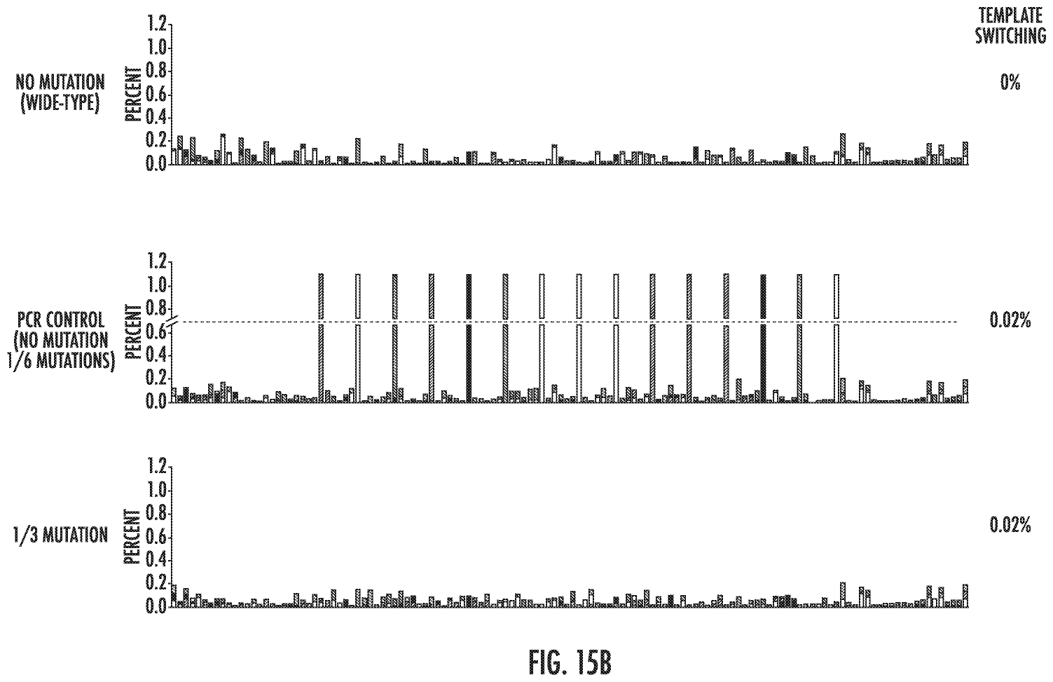

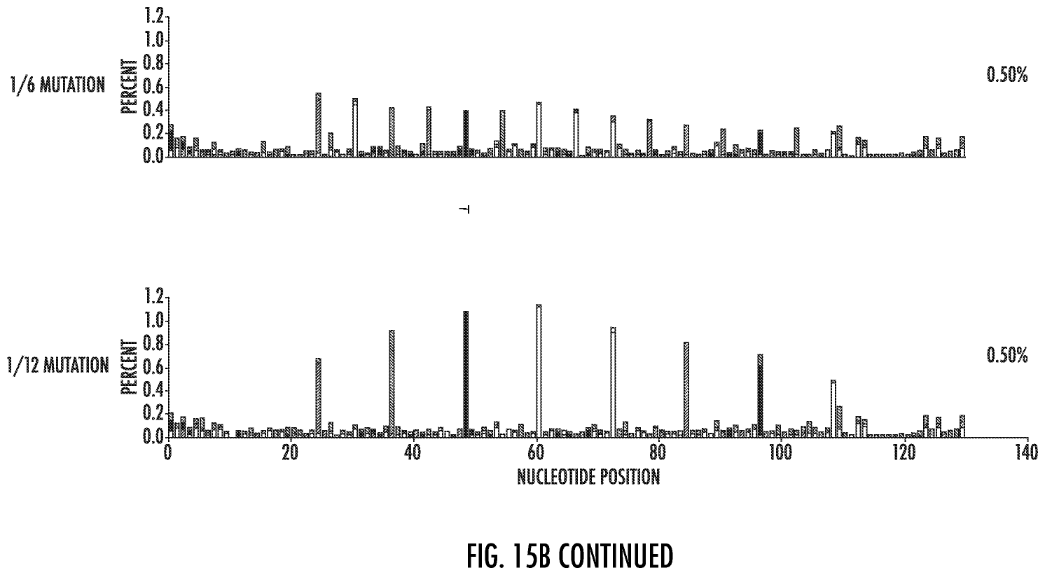

After de-multiplexing of barcoded samples, the 3′ adaptor and all downstream nucleotides were trimmed from the forward reads using Cutadapt (Martin, M., 17(1) EMBNET.JOURNAL 10-12 (2011), and the resulting sequences were mapped to the insert+Lamin A/C locus using Bowtie 2 (Langmead, B. & Salzberg, S. L., 9(4) NAT. METHODS 357-59 (2012)). After removing reads that did not fully map to the template and low-quality reads (Q score less than 35; error probability of 0.00032), sequences were parsed for template switching. To score template switches, we evaluated sequencing reads at diagnostic positions and determined whether each position matched the sequence of the wild-type or mutated template. Reads with a diagnostic nucleotide that did not match either the wild-type or mutated template were discarded. Because the PCR control sample contained a mixture of the fully wild-type and fully mutated templates, we used the first diagnostic position (from the right side of the insert) only as an “anchor” to determine the initial identity of the template; this position was not used to score switching. Thereafter, whenever two or more contiguous diagnostic nucleotides indicated a switch in template identity, we scored this as a switch. For the control sample in which both templates were wild-type, we used the “1/6” mutated template for comparison, to determine the rate of false-positive switches in the assay. Because the PCR control experiment was performed with the wild-type and “1/6” mutated template (FIG. 15 and Tables 1-3), we also used the “1/6” mutated template for scoring switches in this sample. See Table 15 for details.

Cas9 RNP Injection in Mouse Zygotes.

All mouse experiments were carried out under protocols approved by the JHU animal care and use committee. The PCR fragment donor was synthesized as described in Paix et al. (2017). The plasmid donor was generated using a gBlock and restriction enzyme cloning, and purified by Qiagen midi-prep kit and eluted in injection buffer (10 mM Tris-HCl, pH 7.5, 0.1 mM EDTA). Pronuclear injections of zygotes (from B6SJLF1/J parents (Jackson labs)) was performed by the JHU Transgenic facility at a final concentration: 30 ng/μl Cas9 protein (PNABio), 0.6 μM each of crRNA/TracrRNA (Dharmacon) and PCR donor (3 ng/μl or 5 ng/μl) or plasmid donor (10 ng/μl). The Cas9 protein, crRNA, tracrRNA were combined from stocks at 1000 ng/μl, 20 μM, 20 μM respectively and incubated at 4° C. for 10 minutes. Then injection buffer was added to dilute to the final working concentrations above (Tables 1-3) along with repair vector or fragment. The solution was microcentrifuged 5 min at 13000×g and the solution used for injection. Pups were genotyped using genomic primers immediately outside of the PCR donor sequence, or using one primer in mCherry and one upstream of the 483 bp homology arms in the case of the plasmid donor. Genomic DNA from all pups was also subjected to PCR amplification with internal mCherry specific primers to identify random insertions of the donor template (locus-specific mCherry negative/internal mCherry product positive).

We identified 7 pups (11%, out of 60 pups without mCherry insertion at the Adcy3 locus) with potential transgenic insertions of the PCR fragment at other undetermined loci. In contrast, we identified no transgenics (0%, out of 20 pups without mCherry insertion at the Adcy3 locus) when using the plasmid donor.

### Results

mCherry-Tagging of a Mouse Locus Using a PCR Donor with Short Homology Arms.

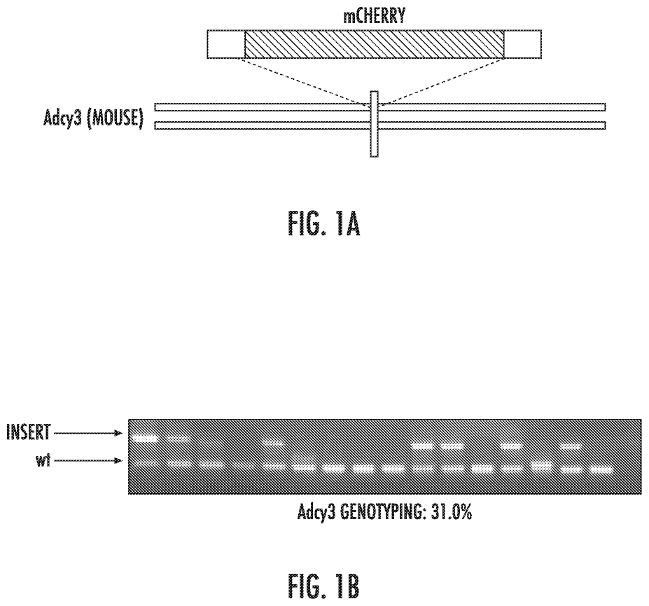

In mammalian systems, ssODNs and plasmids are most commonly used as donors for genome editing (Danner et al., 28(708) MAMMALIAN GENOME 262-74 (2017). To test whether PCR fragments with short homology arms can also function as donors, we designed a PCR fragment to insert mCherry near the C-terminus of the mouse adenylyl cyclase 3 (Adcy3) locus. The mCherry open reading frame (739 bp) flanked by 36 bp homology arm sequences (HS) for the Adcy3 locus was amplified by PCR. The purified PCR fragment and in vitro assembled Cas9 complexes were co-injected into mouse zygotes, and the resulting pups were genotyped by PCR and Sanger sequencing (FIG. 1). We identified 27/87 pups with a correct size insertion at the Adcy3 locus (31% editing efficiency). Sequencing of 10 full-size mCherry edits revealed them all to be precise (no indels). A parallel editing experiment using an mCherry supercoiled plasmid with 500 bp HS yielded 5 edits from 25 pups (20% editing efficiency). Similar knock-in efficiencies have also been reported using long single-stranded donors (Quadros et al., 18(1) GENOME BIOL. 92 (2017)). These results suggest that single-stranded DNAs, plasmids and PCR fragments function with similar efficiency for genome editing in mouse embryos. Unlike single-strand DNAs and plasmids, PCR fragments have the added convenience of ease of synthesis especially for long inserts.

GFP-Tagging of Human Loci Using PCR Donors with Short Homology Arms.

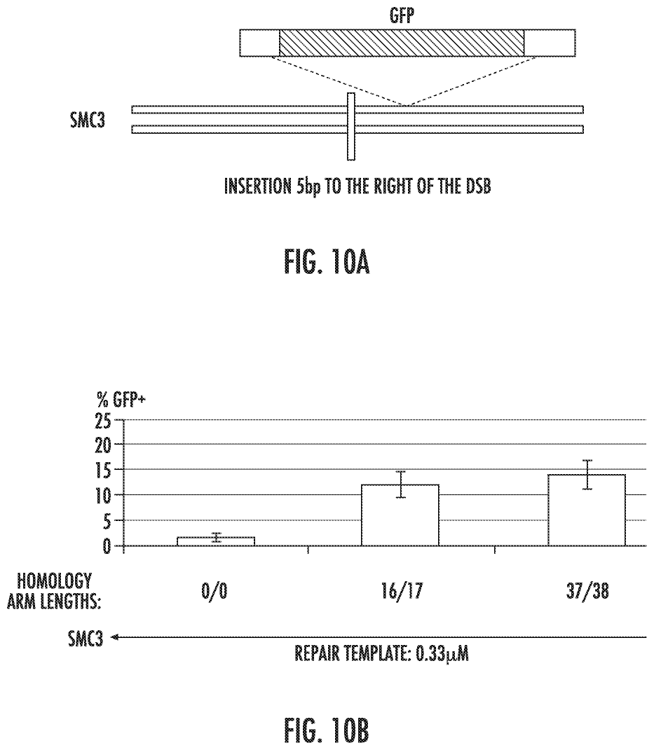

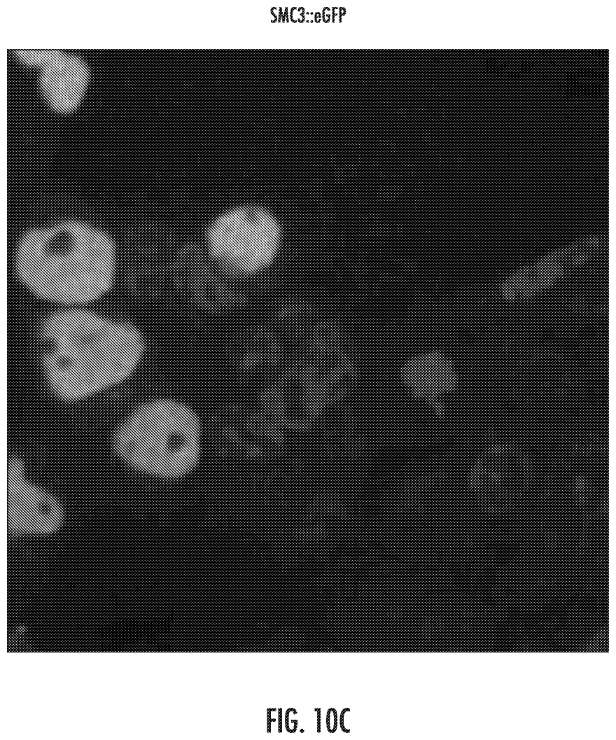

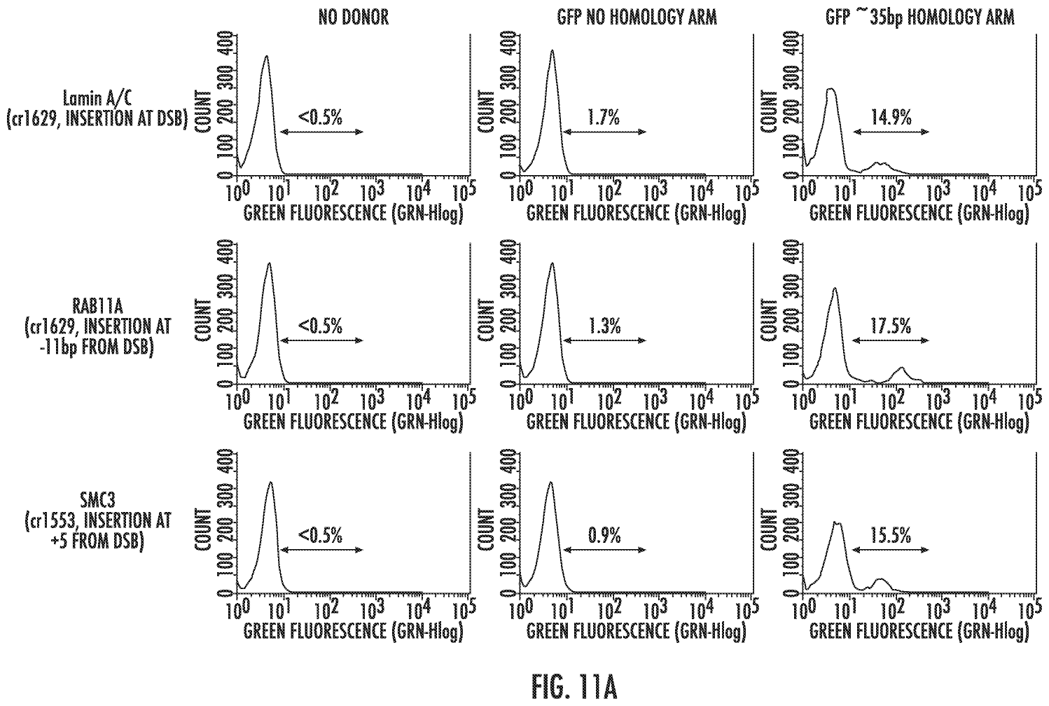

To determine whether PCR fragments can also function for genome editing in human cells, we attempted to knock-in GFP at three loci in HEK293T cells. We designed the HS to insert GFP 0, 11 and 5 bp away from a Cas9 cleavage site in the Lamin A/C, RAB11A, and SMC3 ORFs, respectively (FIGS. 2 and S2). The PCR fragments (0.33-0.21 μM) and in vitro-assembled Cas9-guide RNA complexes were introduced by nucleofection into HEK293T cells without selection as in Leonetti et al. (2016). The efficiency of GFP integration was examined 3 days later by cytometer or fluorescence microscopy. These methods permit the scoring of >5000 cells (cytometer) and >1000 cells (fluorescence) per each nucleofection experiment, and we performed at least two independent experiment for each condition. We obtained an average of 14.9%, 17.5% and 14.0% GFP+ positive cells for the Lamin A/C, RAB11A and SMC3 loci, respectively (FIGS. 2B and 10B). In each case, the cells expressed GFP in a pattern consistent for the targeted ORF (FIGS. 2D and 10C).

Reducing the molarity of the PCR fragments by 10-fold reduced efficiency by ˜½ (Compare FIGS. 2B and 2C). Increasing the length of the homology arms to 500 bp did not increase editing efficiency, even when controlling for the reduced molarity of the longer PCR fragments (FIG. 2C). Reducing the length of the homology arms to ˜15 bp, however, decreased efficiency (FIG. 2B). PCR fragments with no homology sequence or homology arms for a locus not targeted by Cas9 yielded GFP+ positives in the range of the background levels obtained with cells that did not receive any repair template (FIGS. 2, 10, 11 and Table 1). Plasmid donors with ˜500 bp homology arms also performed poorly (FIG. 2C) as reported previously (He et al., 44(9) NUCL. ACIDS RES. E85 (2016)). We conclude that PCR fragments function as efficient donors in HEK293T cells, performing better than plasmids with much longer homology arms. Because −35 bp homology arms are convenient to introduce by PCR amplification, we used that length for subsequent experiments. 30-40 nt homology arms have also been reported to be optimal for ssODNs (Liang et al., 241 J. BIOTECHNOL. 136-46 (2017)).

Editing Efficiency is Sensitive to Insert Size.

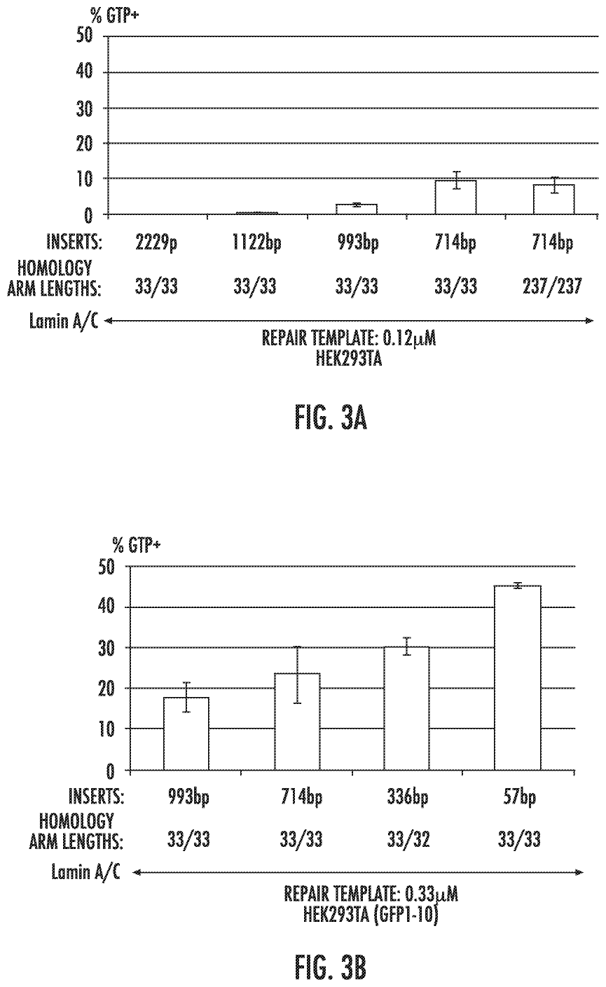

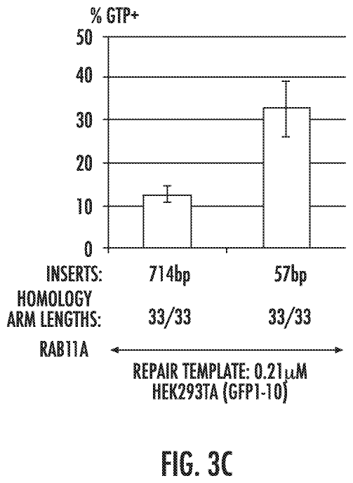

To test the effect of insert size on editing efficiency, we added varied sizes of DNA sequence to the GFP insert. For ease of synthesis and to maintain equimolar amounts of donor DNAs, we introduced donor fragments at the same low molarity (0.12 μM). We found that inserts beyond 1 kb performed very poorly, yielding less than 0.5% edits (FIG. 3A). By varying the size of the homology arms, we found that the size of the insert, and not the overall size of the donor DNA, determines editing efficiency. An 1188 bp donor (714 bp insert with two 237 bp HS) performed as well as a 780 bp donor with the same size insert and 33 bp HS (8.5% versus 9.8% edits, FIG. 3A). The 1188 bp donor, however, performed much better than an 1188 bp donor with a longer insert (1122 bp) and 33 bp HS (8.5% versus 0.3% edits, FIG. 3A).

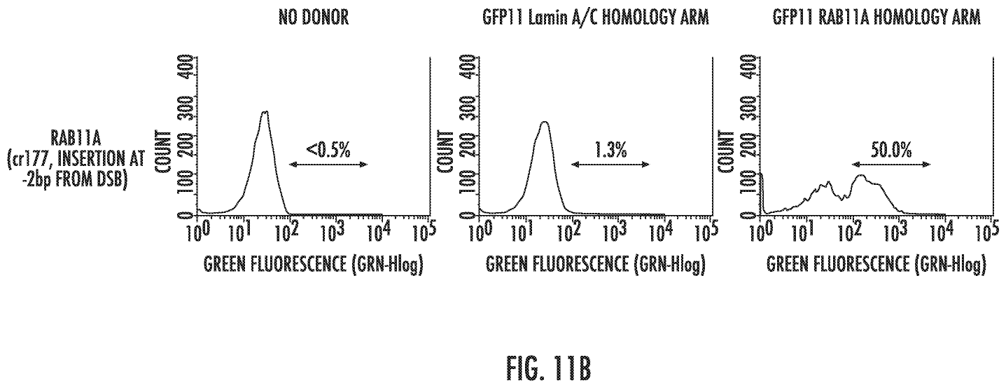

To test whether decreasing insert size below the size of GFP would increase editing efficiency, we took advantage of the split-GFP system (Kamiyama et al. (2016; Leonetti et al. (2016)). In this system, the 11^{th} beta-strand of GFP (57 bp, GFP11) is knocked-in in cells expressing a complementary GFP fragment (GFP1-10). We generated PCR products containing the GFP11 insert and ˜35 bp HS and introduced these at 0.33 μM. We obtain 45.4% edits at the Lamin A/C locus (FIG. 3B) and 32.8% at the RAB11A locus (FIG. 3C). A donor with no homology arm yielded only 1.3% edits (FIG. 11B). Again, we found that increasing insert size reduced efficiency, down to 17.9% for a 993 bp insert (FIG. 3B). We conclude that dsDNAs engage in an efficient repair process that requires only 35 bp homology arms, but favors relatively short inserts (<1 kb at the molarities tested here).

Accuracy of Repair is Asymmetric.

To investigate the accuracy of repair with PCR fragments, we isolated GFP+ and GFP− cells by fluorescence-activated cell sorting from a single editing experiment targeting the Lamin A/C locus with a GFP-containing PCR fragment under optimal conditions (FIG. 2B, 33/33 HS, 0.33 μM molarity). Each cell was grown out as a clone and the Lamin A/C locus was amplified using two primers flanking the insertion site. As expected, all 48 GFP+ clones contained at least one Lamin A/C allele with a full-size insert (4 were homozygous with two edited alleles). We sequenced the GFP insert in 23 of the 48 GFP+ clones and identified 20 precise insertions and 3 imprecise insertions containing small in-frame indels at the left or right junction (FIGS. 12 and 13). We also sequenced the wild-type-sized allele in 11 of the 44 heterozygous GFP+ clones, and identified 2 with wild-type sequence, 6 with indels at the DSB, and 3 with small inserts (<100 bp) corresponding to either the N-terminus or C-terminus of GFP (FIG. 13). We also screened 37 GFP− clones by PCR and, surprisingly, identified 10 that contained inserts at the Lamin A/C locus. We sequenced 7 of the 10 inserts and identified 3 with a full-size GFP insert with out-of-frame indels at one junction and 4 with smaller GFP inserts (FIG. 13).

In total, we sequenced 13 imprecise GFP edits and found only one internal deletion and one insertion in the wrong orientation (FIG. 13). All other imprecise edits were full-size or truncated GFP fragments inserted in the correct orientation. All had one precise junction on the non-truncated terminus of GFP. The other junction was imprecise and contained indels (FIG. 13). These observations are consistent with an asymmetric repair process that uses mechanisms with different homology requirements to initiate and resolve repair.

Repair is a Polarity-Sensitive Process.

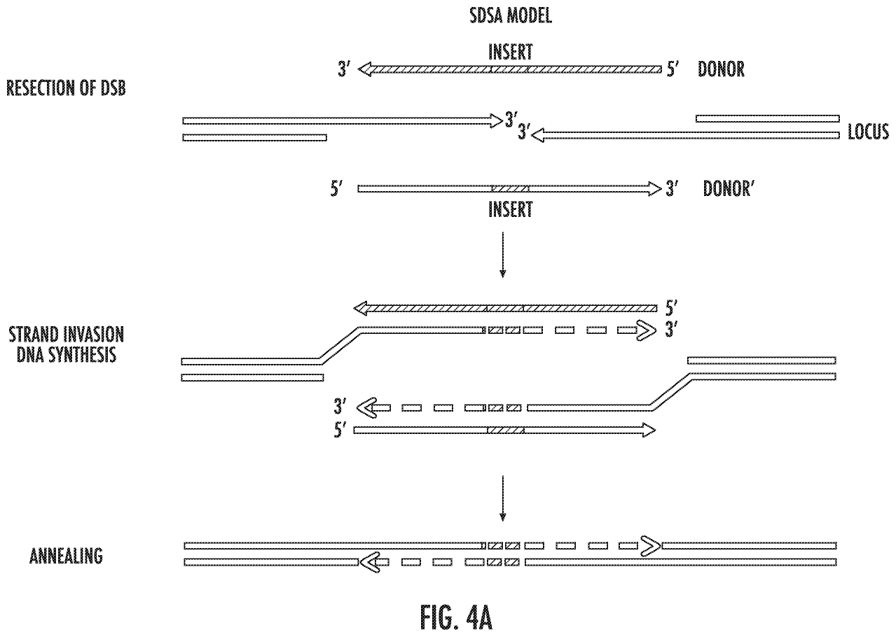

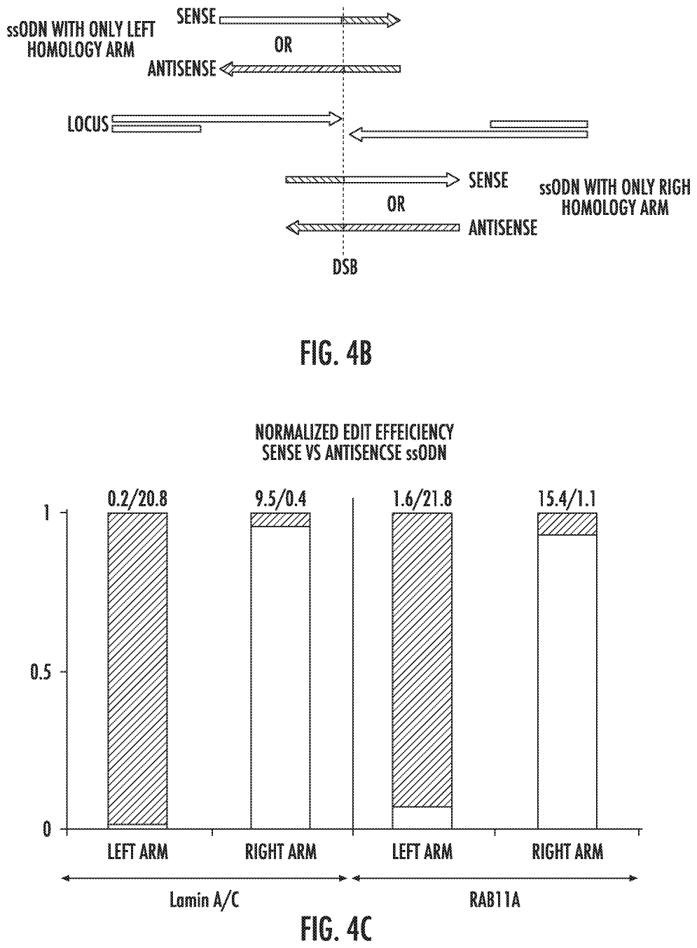

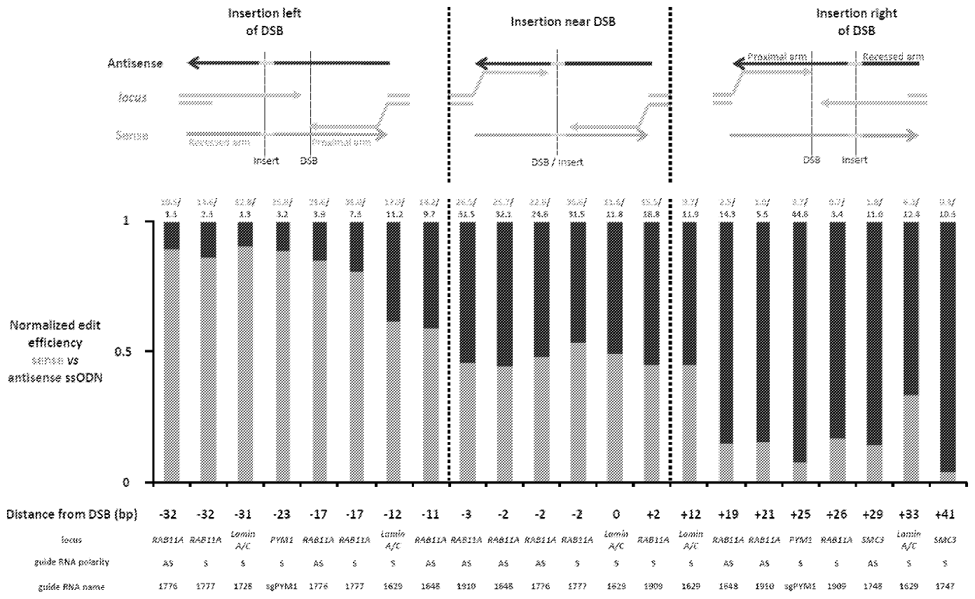

In the SDSA model, initiation and resolution of repair proceeds via distinct steps. First, the DSB is resected to yield 3′ overhangs on both sides of the DSB (FIG. 4A). The 3′ overhangs pair with the donor and are extended by DNA synthesis copying donor sequences (FIG. 4A). Bridging of the DSB is completed when the newly synthesized strands withdraw from the donor and anneal back at the locus (FIG. 4A). To determine whether initiation and resolution might have different homology requirements, we tested the editing efficiency of single-stranded donors (ssODNs) bearing only one HS. We designed ssODNs with a GFP11 insert and only one HS at either the 3′ or 5′ end of the ssODN (5′ or 3′ HS). The HS targeted sequences on the left or right side of the Cas9-induced DSB in Lamin A/C and RAB11A (FIG. 4B). At both loci, we found that editing efficiency was highest with ssODNs that had a 3′ HS that could anneal to a complementary 3′ end at the DSB (FIG. 4C). ssODNs of the opposite polarity yielded only background-level edits. These observations are consistent with a replicative repair process that requires pairing between a 3′ HS on the donor and sequences on at least one side of the DSB. Apparently, a different, less stringent mechanism can be used to bridge the donor to the other side. One possibility is that NHEJ was used to repair the gap on the side with no HS. Coupling of homologous and non-homologous repair mechanisms has already been documented in mammalian cells (Richardson, C. & Jasin, M., 20(23) MOL. CELL. BIOL. 9068-75 (2000)).

Polarity of Single-Stranded Donors Affects Incorporation of Distal Edits.

We wondered whether the different requirements for homology on the 3′ and 5′ ends of single-stranded donors might also apply to donors that contain two HS at different distances from the DSB. Such HS are found in donors designed to insert an edit at a distance from the DSB. In these donors, one HS (proximal HS) matches sequences immediately next to the DSB and the other HS (recessed HS) matches sequences at a distance from the DSB on the distal side of the edit (FIG. 5A). We tested whether proximal and recessed HS function equivalently on the 5′ and 3′ ends of ssODNs using a series of 23 pairs of sense and antisense ssODNs with inserts ranging from 0 to 41 nucleotides from the DSB at four loci (FIG. 5B and Table 3). (In all ssODNs, the sequence between the DSB and edit was partially recoded to promote edit incorporation as described in the next section). Strikingly, we observed an increasing bias for a particular polarity with increasing edit-to-DSB distance (FIG. 5B). The favored ssODN polarity changed whether the edit (and recessed homology arm) was positioned to the left or right of the DSB (sense polarity when the edit is on the left side of the DSB, and antisense when the edit is on the right side). ssODNs with inserts close to the DSB did not show much polarity bias (FIG. 5B). These findings demonstrate that repair favors ssODNs with 3′ HS that directly abut the DSB (proximal HS) and suggest that initiation of repair synthesis is enhanced by donors that can pair with sequences directly flanking the DSB. These experiments also showed that, in contrast to ssODN polarity, the polarity of the guide RNA used to create the DSB had no discernable effect on editing efficiency (FIG. 5B). We conclude that, under the conditions used here, the requirements for replicative repair have a greater impact on editing efficiency than the strand-bias imposed by asymmetric Cas9 release of the DSB (Richardson et al. (2016)).

Recoding of Sequences Between the DSB and the Edit Increases Recovery of Distal Edits.

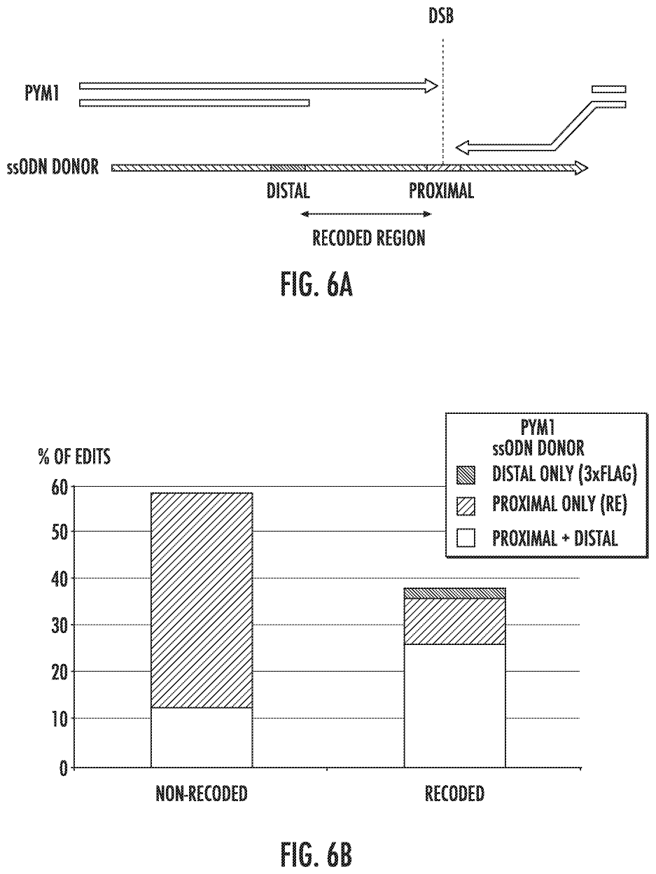

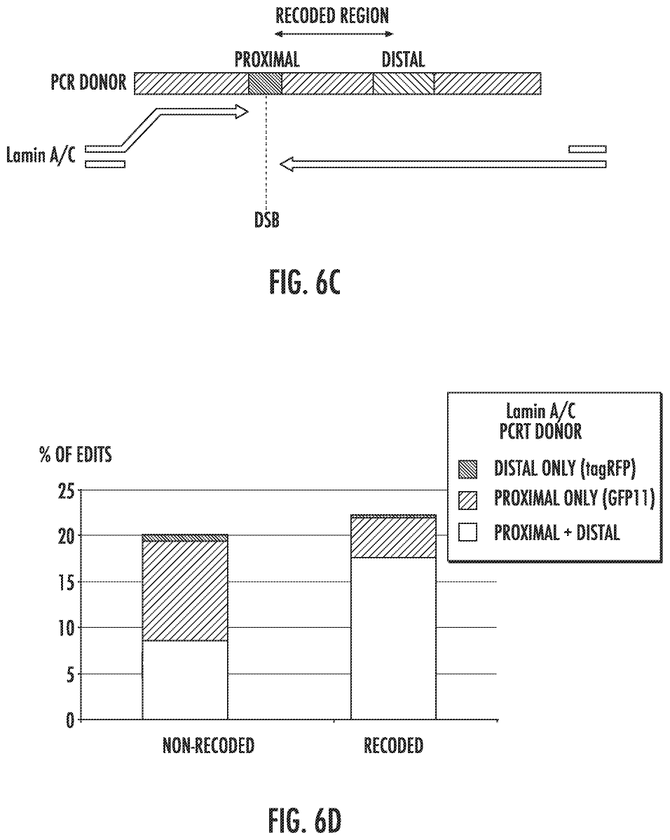

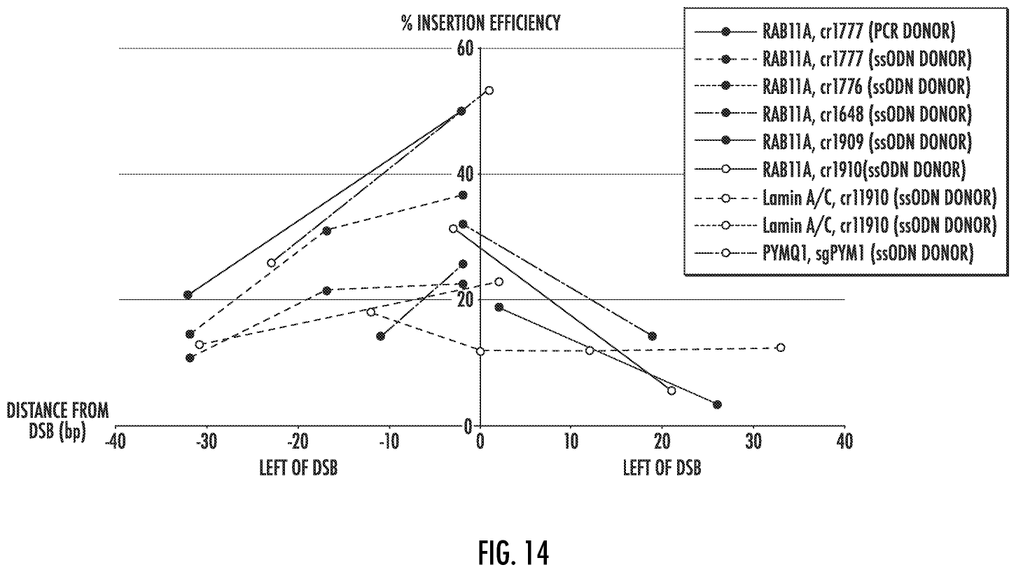

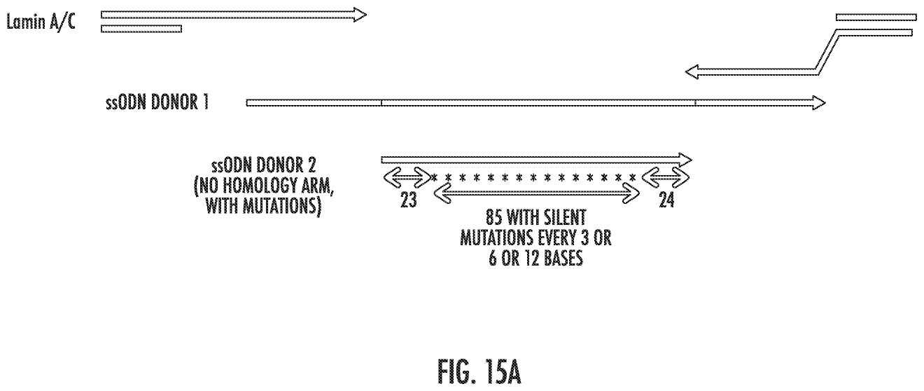

Editing efficiency has been observed to decrease with increasing distance between the edit and the DSB (Paquet et al., 533(7601) NATURE 125-29 (2016)). This observation is also consistent with replicative repair, which predicts that synthesis that generates sequence complementary to the other side of the DSB will promote annealing back to the locus, potentially even before the edit is copied (FIG. 6). To test this prediction directly, we designed an ssODN donor with two inserts: a proximal insert (restriction enzyme site) one base away from the DSB in the PYM1 locus and a distal insert (3×Flag) 23 bases away from the DSB. Each insert was flanked by an HS targeting the PYM1 locus (FIG. 6A). We generated 63 single cell clones and genotyped the PYM1 locus by PCR (see Material and Methods). 46% of the clones contained only the proximal edit and 12.6% contained both the proximal and distal edits (FIG. 6B). The finding that ˜80% of the edits contained only the proximal edit is consistent with annealing using sequence between the two edits. To test this hypothesis, we mutated 7 bases in the 23 bases region separating the proximal and distal edit. The mutations were designed to reduce homology with the locus while preserving coding potential (FIG. 6A). This partial recoding reduced the frequency of proximal edit-only clones to 10.3% and increased the frequency of proximal+distal edits to 25.8% (FIG. 6B). We conclude that sequences on the donor that span the DSB can prevent incorporation of distal edits. We note that, although recoding enhances the recovery of distal edits, recoding does not eliminate the preference for proximal edits, which are still recovered at higher frequency than distal edits even when using recoded templates (FIG. 14).

To test whether internal homologies can also participate in the repair process when using double-stranded donors, we performed a similar experiment with a PCR fragment designed to incorporate GFP11 at the DSB, and tagRFP 33 bases from the DSB in the Lamin A/C locus (FIG. 6C). We recovered 10.8% GFP-only edits and 8.6% GFP-RFP double positives (FIG. 6D). Partial recoding of the sequence between GFP11 and tagRFP (by introducing 10 silent mutations) reduced the percent of GFP-only edits to 4.4% and raised the percent of GFP-RFP double positives to 17.6% (FIG. 6D). We conclude that internal homologies on double-stranded templates can also interact with the targeted locus. Since both polarities are present in double-stranded templates, internal sequences could participate in principle in both the initial invasion step and the annealing step back to the locus.

Repair is Prone to Template Switching Between Donors.

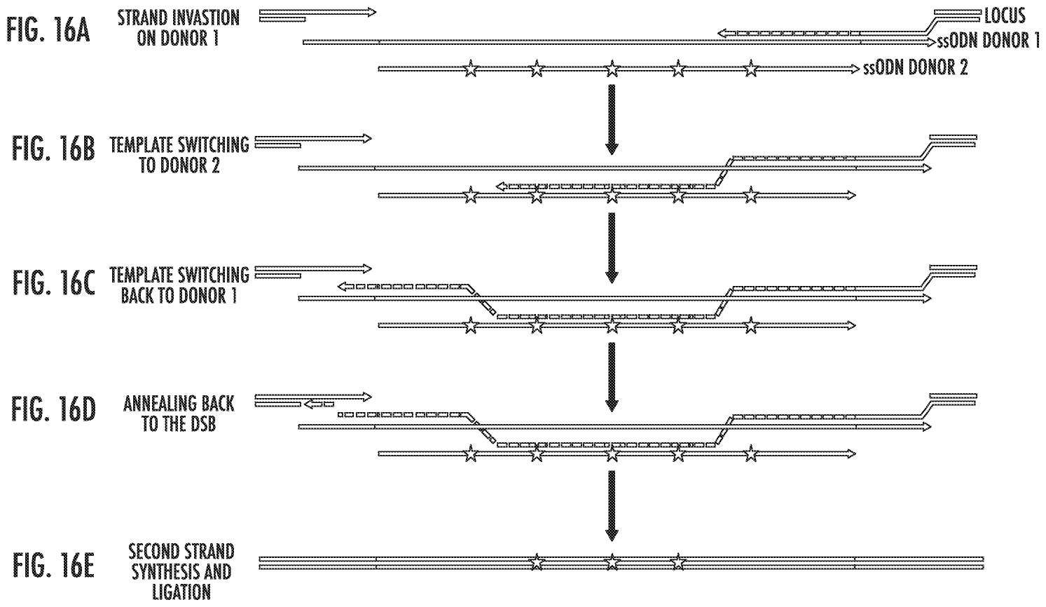

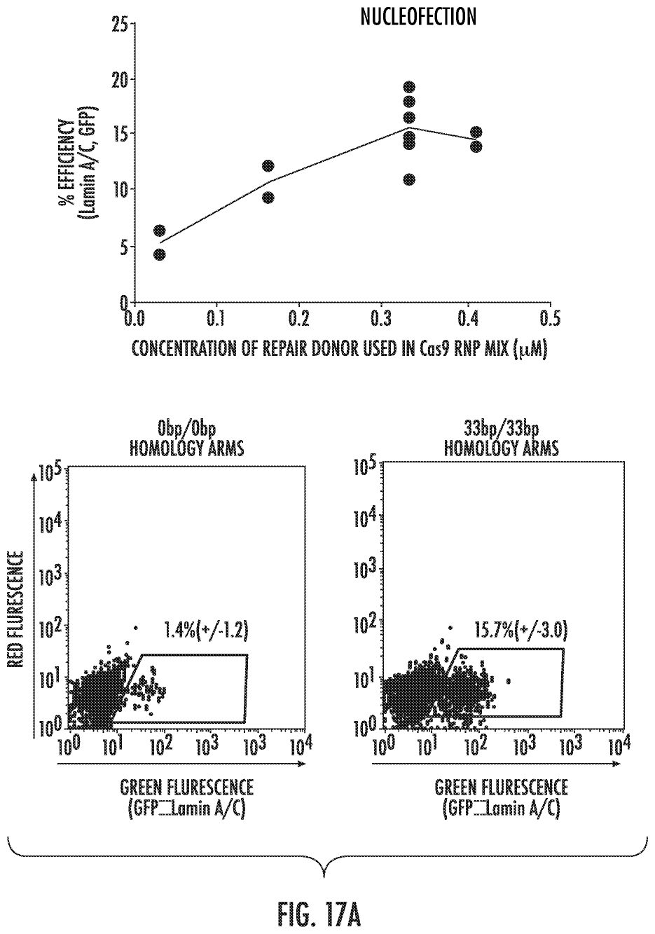

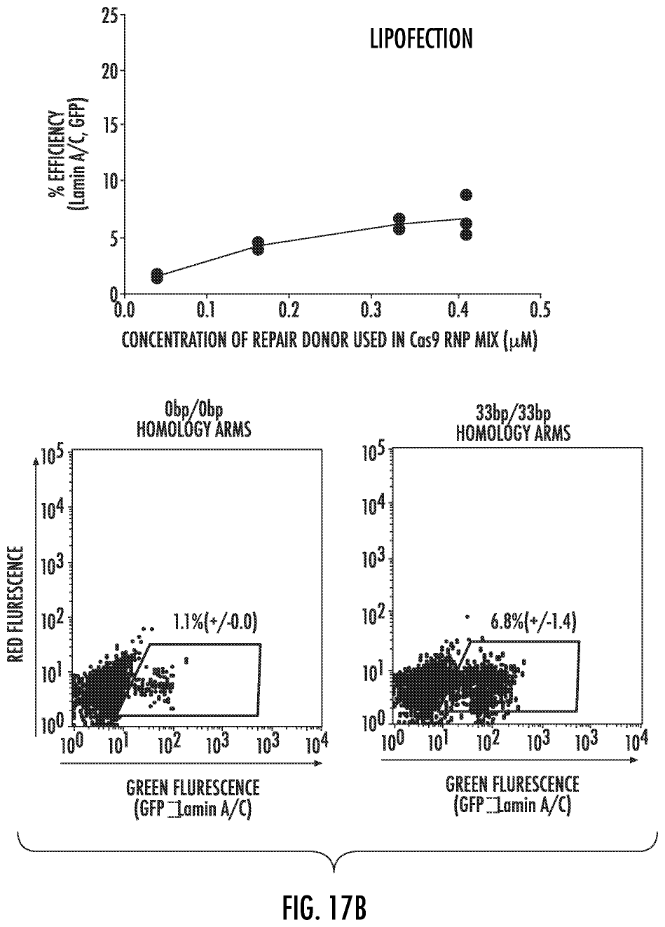

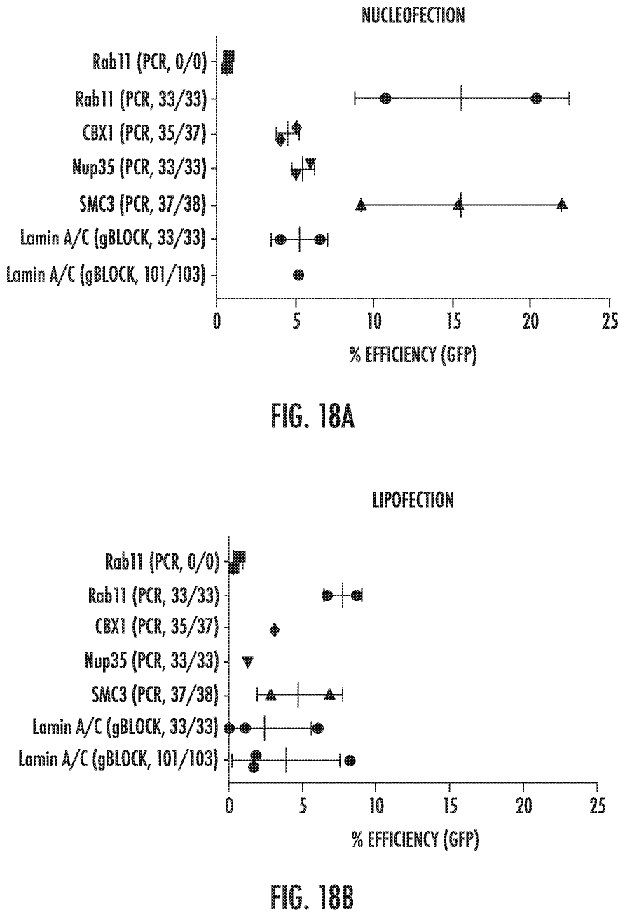

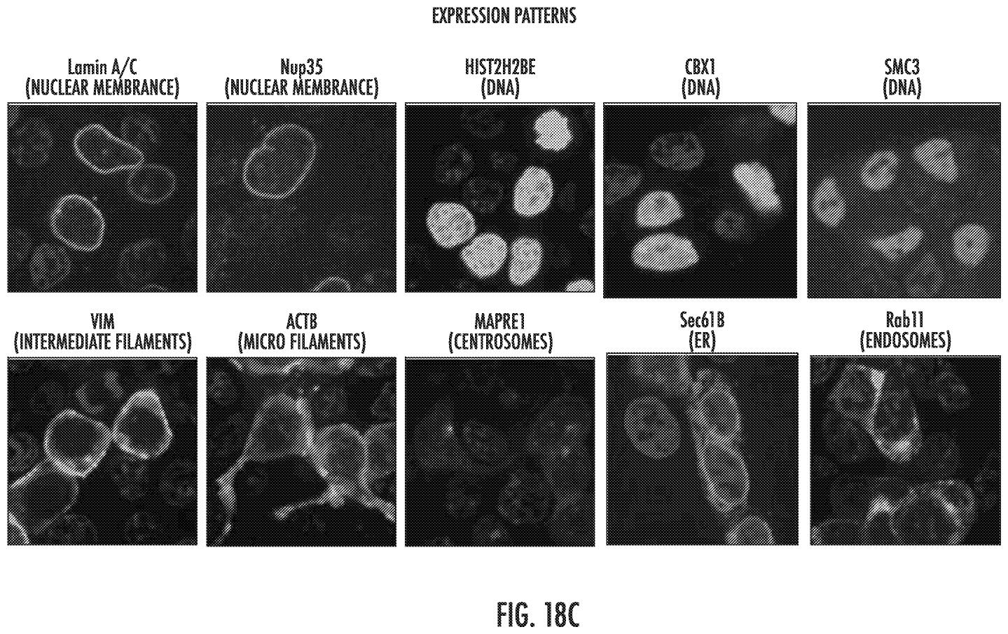

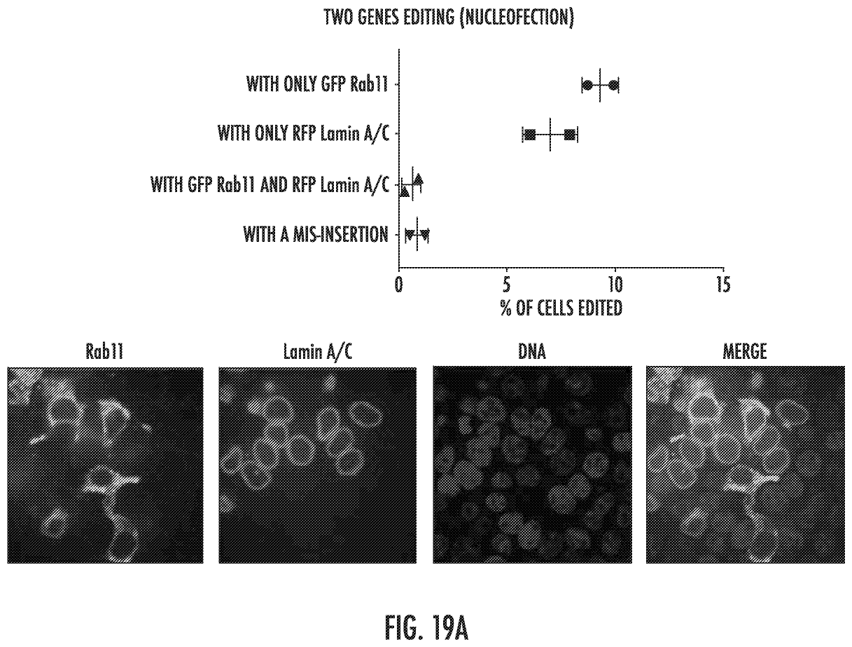

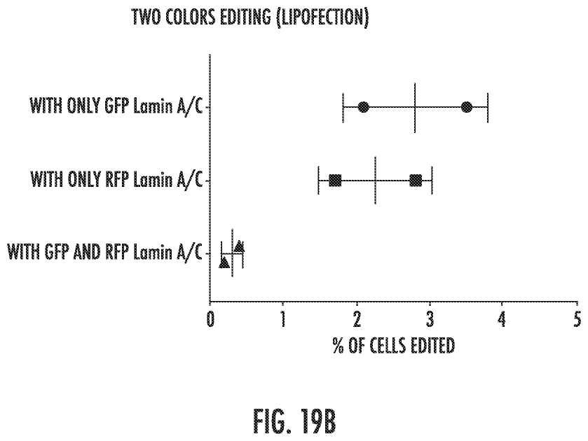

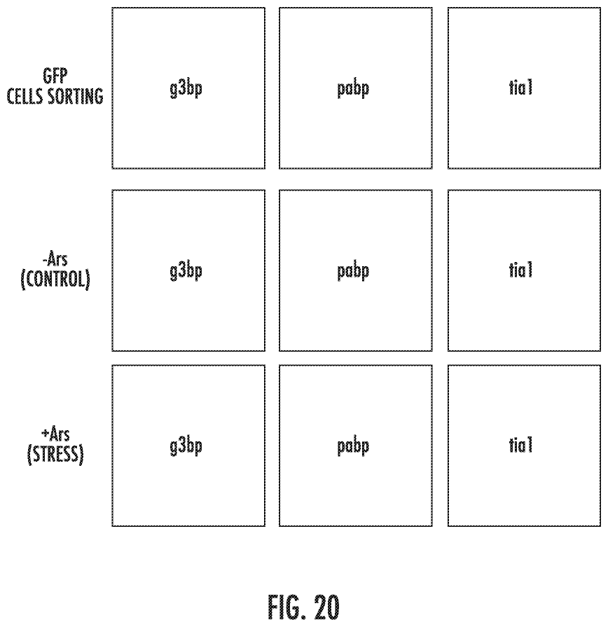

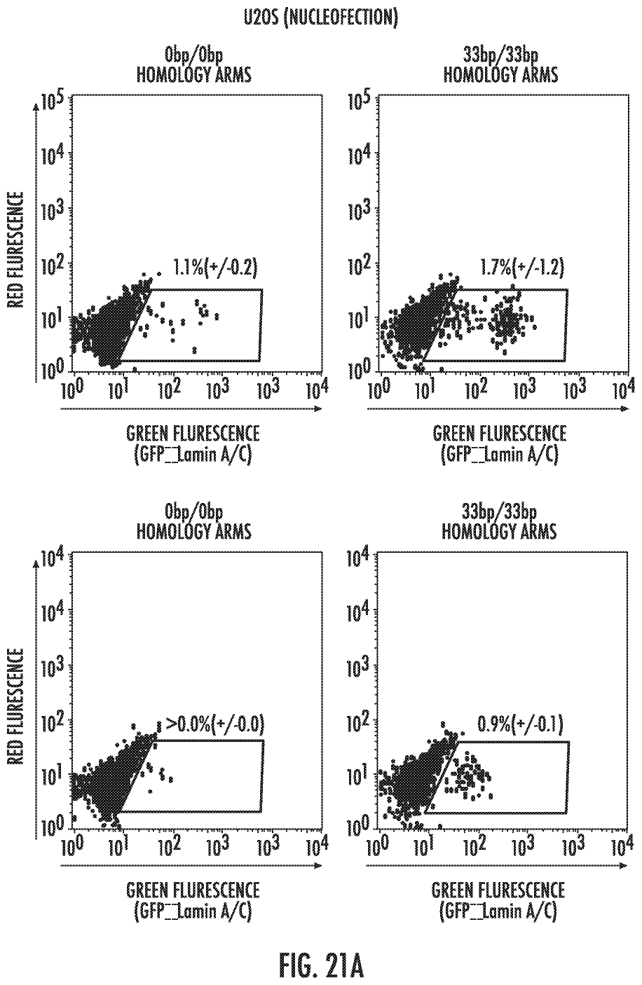

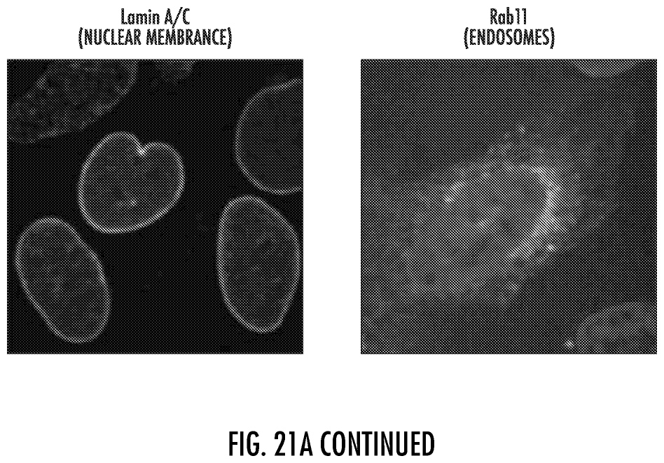

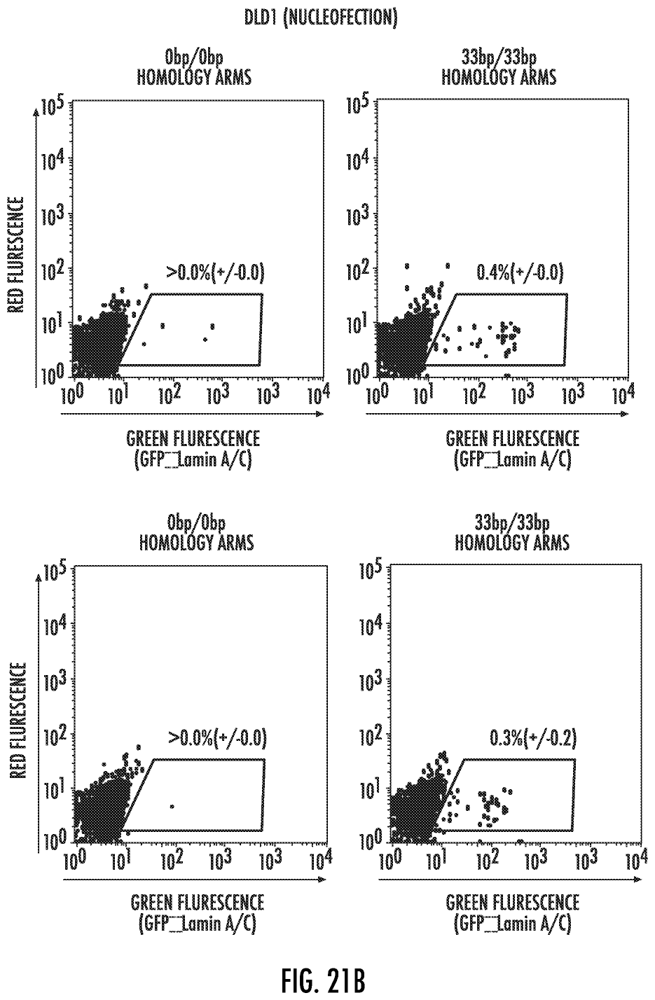

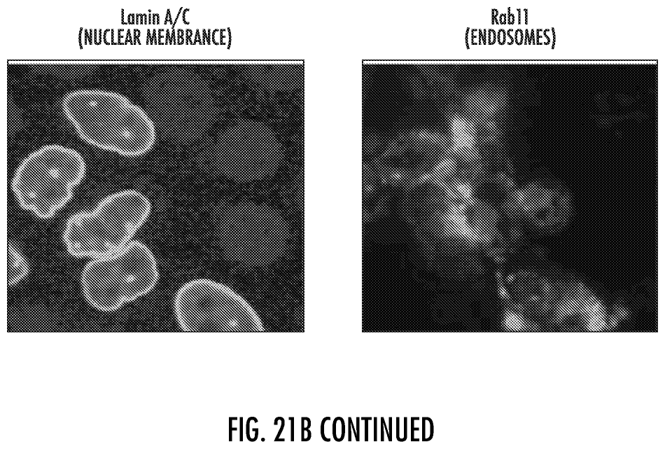

Another characteristic of SDSA first observed in yeast is the ability of the repair process to undergo sequential rounds of invasion and synthesis (29, 30). “Template switching” can create edits that combine sequences from overlapping donors (14). To test whether template switching also occurs in human cells, we used two donors to correct a single DSB. The first donor was an ssODN with two HS and a GFP11-coding insert containing a STOP codon to prevent translation of the full-length fusion (FIG. 7A). The second donor was an ssODN with the same GFP11 insert but without the STOP codon and without any HS. Consistent with template switching, we obtained 3.2% GFP+ edits when using both donors, compared to 0.3% and 0.4% GFP+ edits when using only the first or second ssODN, respectively (FIG. 7B). We repeated this experiment with double-stranded donors and obtained similar results (FIG. 7C/D). We conclude that template switching between donors can occur in human cells (FIG. 16).

To visualize template switching more directly, we combined wild-type donors with recoded donors where the GFP11 insert contained several silent mutations and used Illumina sequencing to sequence the insertional edits en masse (FIG. 7E). Using recoded donors with silent mutations every 12 bases in the GFP11 insert, we identified evidence of template switching in 1.4% of edits (“chimeric edits”). Interestingly, the same experiment performed with donors that contained silent mutations every 6 or every 3 nucleotides resulted in only 0.5% and 0% chimeric edits, respectively (FIGS. 7F and 15, Table 15). The chimeric edits could not have resulted from sequential rounds of Cas9 cleavage and repair, since the edit destroyed the crRNA pairing sequence. The chimeric edits also could not have arisen during PCR amplification, since we observed no chimeric edits in a control experiment mixing two different cell populations (FIG. 15). We conclude that template switching occurs between donors in human cells and is sensitive to the degree of homology between donors (FIG. 16), as reported previously in yeast (Anand et al., 28(21) GENES DEV. 2394-2406 (2014) and Tsaponina, O. & Haber, J. E., 55(4) MOL. CELL. 615-25 (2014)).

### Discussion

In this report, we demonstrate that PCR fragments are efficient donors for genome editing in mouse embryos and human cells. PCR fragments with short homology arms (HS ˜35 bp) can be used to integrate edits up to 1 kb, long enough to encode fluorescent reporters such as GFP. Experiments using single and double-stranded DNAs suggest that linear donors participate in a replicative repair mechanism that broadly conforms to the SDSA model for gene conversion. Our findings suggest simple guidelines to streamline donor design and maximize editing efficiency (FIG. 8).

Linear DNAs Repair Cas9-Induced DSBs by Templating Repair Synthesis.

In principle, linear donors could repair Cas9-induced breaks by integrating directly at the DSB. For example, microhomology-mediated end-joining (MMEJ) could cause donor ends to become ligated to each side of the DSB (Yao et al., 20 EBIO MEDICINE 19-26 (2017)). Alternatively, HS on the donor could form holiday junctions with sequences on each side of the DSB. Cross-over resolution of the two holiday junctions could cause donor sequences to become integrated at the DSB. This type of HDR has been proposed to underlie genome editing with plasmid and viral donors (Kan et al., 27(7) GENOME RES. 1099-1111 (2017)). In these models, repair is symmetric: the same mechanism (MMEJ or recombination) is used to ligate donor sequences to each side of the break. In contrast, our observations suggest that repair with linear donors proceeds by an asymmetric, likely replicative, process. First, ssODNs with only one HS show strong polarity specificity (FIG. 4C), consistent with a specific requirement for pairing with 3′ ends at the DSB (FIG. 4A). Second, recessed HS (HS at a distance from the DSB) are rarely used to initiate repair synthesis, but can be used to resolve a repair event (FIGS. 5 and 6). Third, internal homologies on the donor can bypass integration of distal edits (FIG. 6). Fourth, most imprecise edits have asymmetric junctional signatures (FIG. 13). These observations suggest that the repair process is polar, like DNA synthesis, and has different requirements to initiate and resolve repair. These findings are consistent with the SDSA model for gene conversion (Paques, F. & Haber, J. E., 63(2) MICROBIOL. MOL. BIOL. REV. 349-404 (1999)) (FIG. 4A). SDSA initiates with DNA synthesis templated by the donor to extend 3′ ends at the DSB, and resolves by annealing of the newly replicated strand(s) back to the locus. Our observations suggest that initiation of DNA synthesis is the most homology-stringent step, requiring a ˜35 base HS on the donor complementary to sequences directly adjacent to one side of the DSB. Either side of the DSB can initiate repair and, contrary to an earlier report (Liang et a. (2016)), we did not observe a preference consistent with biased strand-release by Cas9. The observations that HS longer than 35 bases do not perform significantly better, and that distal HS perform more poorly, also suggest that resection exposes only short regions of ssDNA on either side of the DSB. In contrast to the initiation step, the resolution step has more relaxed homology requirements. Recessed homology arms can be used for that step, and in fact repair can proceed with no HS on the “annealing side” (FIG. 4C). In that case, NHEJ (or MHEJ) may be used to fuse the newly replicated strand to the other side of the DSB. One possibility is that NHEJ or MHEJ competes with annealing during resolution, especially in the case of long edits where synthesis has a higher chance of stalling before reaching the distal HS or before synthesis of a complementary strand primed from the other side of the DSB (FIG. 4A). Consistent with this view, we recovered several partial GFP insertions that were integrated in the correct orientation but contained one imprecise junction on the truncated side of GFP, consistent with premature withdrawal from the donor. We cannot exclude the possibility, however, that in these partial edits, the non-homologous joint was made first using a broken donor.

If partial edits are due to premature withdrawal of the newly replicated strand from the donor, partial edits should be less frequent when using donors with shorter inserts. Consistent with this prediction, we found that editing efficiency is inversely proportional to insert size. At the Lamin A/C locus, we obtained 45.4% edits for a 57 bp insert, 23.5% edits for 714 bp insert (GFP) and 17.9% edits for a 993 bp insert. The size of the insert, and not the overall size of the donor, correlated with efficiency, arguing against the possibility that breakage of longer donors contributes to reduced efficiency (FIG. 3). We suggest that the low processivity of repair polymerases (Parsons et al., 18(8) ANTIOID. REDOX SIGNAL 851-73 (2013)) increases the chances of aberrant dissociation/annealing events on long inserts.

We also obtained evidence for dissociation and invasion events between donors. Such “template switching” was also observed in yeast and C. elegans and can cause sequences from overlapping donors to become incorporated in the same edit (Anand et al. (2014); Tspaonina et al. (2014)) (Paix et al., 44(15) NUCLEIC ACIDs RES. e128 (2016)). We found that template switching is sensitive to the degree of homology between donors and is reduced significantly by mutations every 3 or 6 bases, as was also found in yeast (Anand et al. (2014; Tsaponina et al. (2014)). Similarly, recoding of sequences between the DSB and the edit promotes the incorporation of distal edits, presumably by increasing the rejection rate of heteroduplexes formed during annealing between the newly replicated strand and sequences flanking the DSB (Sugawara et al., 101(25) PROC. NATL. ACAD. SCI. USA 9315-20 (2004)). Template switching may also explain why editing efficiency is sensitive to donor molarity, since high donor molarity is predicted to lower the frequency of aberrant dissociation/re-annealing events during synthesis. It will be interesting to determine which repair polymerases are responsible for synthesis templated by linear donors and whether their processivity characteristics account for our observations of template switching. In this regard, it is interesting to note that we identified a higher frequency of full-length edits (and lower frequency of partial edits) in mice compared to HEK293T cells. This difference could reflect differences in the properties of the enzymes that mediate SDSA in the two systems. Alternatively, the higher precision in mice could be due to a more efficient method for delivering donors at high molarity (pronuclear injection in mouse zygotes versus nucleofection in HEK293T cells).

SDSA as a Repair Mechanism for Cas9-Induced DSBs: Implications for Genome Editing.

The demonstration that ssODNs and PCR fragments engage in a SDSA-like mechanism to repair Cas9-induced DSBs has two important implications for genome editing. First, the SDSA model makes simple predictions for optimal donor design (FIG. 8). These predictions improve editing efficiencies for edits at distance from the DSB, and eliminate the effort and expense used in creating donor DNAs with unnecessarily long homology arms. Linear donors with short homology arms can be chemically synthesized as single-stranded or double-stranded DNA or PCR amplified, avoiding the need for cloning. In this manner, tagging of genes with GFP can be achieved readily, without resorting to split-GFP approaches that also require expression of a complementary GFP1-10 fragment (Leonetti et al., 113(25) PROC. NATL. ACAD. SCI. USA E3501-08 (2016)). Second, because SDSA is thought to be a widespread mechanism for DSB repair among eukaryotes (Iyama, T. & Wilson, D. M., 12(8) DNA REPAIR (AMST) 620-36 (2013)), it is likely that the approaches outlined here will be applicable to other cell types and organisms. We documented previously that PCR fragments with short HS perform well in C. elegans (Paix et al. (2016)), and we demonstrate here the same for HEK293T cells and mouse embryos. It will be interesting to investigate whether linear donors with short HS can also be used for genome editing in pluripotent cells and post-mitotic cells.

TABLE 1
Detailed experimental conditions and results

| | | | | | | |
|---|---|---|---|---|---|---|
| | | | | crRNA/sgRNA | | |
| | | | | polarity | | Repair template left/ |
| | | | crRNA/ | (relative to | | right homology) arms |
| | | | sgRNA | gene coding | Repair template | (nucleotides/ |
| Edit | Cell type | Cas9 delivery | name) | sequence) | type/name | basepairs) |

FIG. 1

| | | | | | | |
|---|---|---|---|---|---|---|
| [symbol] | Pronuclear | RNP/ | crAdcy3 | AS | Plasmid pBS- | 483/421 |
| | injection of | Injection | | | AC3CtermGenomic- | |
| | mouse embryos | | | | mCherry | |
| [symbol] | Pronuclear | RNP/ | crAdcy3 | AS | PCR 1596/1597 | 36/36 |
| | injection of | Injection | | | | |
| | mouse embryos | | | | | |

FIGS. 2 / 10 / 11

| | | | | | | |
|---|---|---|---|---|---|---|
| [symbol] | HEK293T | RNP/ | crRNA 1629 | S | PCR 1630/832 | 0/0 |
| | | Nucleofection | | | | |
| [symbol] | HEK293T | RNP/ | crRNA 1629 | S | PCR 1685/1686 or | 16/16 |
| | | Nucleofection | | | PCR 1858/1859 | |
| [symbol] | HEK293T | RNP/ | crRNA 1629 | S | PCR 1618/1619 or | 33/33 |
| | | Nucleofection | | | PCR 1743/1744 | |
| [symbol] | HEK293T | RNP/ | crRNA 1629 | S | PCR 1743/1744 | 33/33 |
| | | Nucleofection | | | | |
| [symbol] | HEK293T | RNP/ | crRNA 1629 | S | PCR 1741/1742 | 518/518 |
| | | Nucleofection | | | | |
| [symbol] | HEK293T | RNP/ | crRNA 1629 | S | Plasmid 1716 | 518/518 |
| | | Nucleofection | | | | |
| [symbol] | HEK293T | RNP/ | crRNA 1648 | AS | PCR 1630/832 | 0/0 |
| | | Nucleofection | | | | |
| [symbol] | HEK293T | RNP/ | crRNA 1648 | AS | PCR 1838/1839 | 15/15 |
| | | Nucleofection | | | | |
| [symbol] | HEK293T | RNP/ | crRNA 1348 | AS | PCR 1840/1841 or | 33/33 |
| | | Nucleofection | | | PCR 1652/1653 | |
| [symbol] | HEK293T | RNP/ | crRNA 1648 | AS | PCR 1840/1841 | 33/33 |
| | | Nucleofection | | | | |
| [symbol] | HEK293T | RNP/ | crRNA 1648 | AS | PCR 1846/1847 | 461/432 |
| | | Nucleofection | | | | |
| [symbol] | HEK293T | RNP/ | crRNA 1648 | AS | Plasmid 1791 | 461/432 |
| | | Nucleofection | | | | |
| [symbol] | HEK293T | RNP/ | crRNA 1629 | S | PCR 1652/1653 | No homology arm, PCR |
| | | Nucleofection | | | | contatining eGFP with |
| | | | | | | RAB11A homology arms |
| | | | | | | used with Lamin A/C |
| | | | | | | crRNA |
| [symbol] | HEK293T | RNP/ | crRNA 1553 | AS | PCR 1630/832 | 0/0 |
| | | Nucleofection | | | | |
| [symbol] | HEK293T | RNP/ | crRNA 1553 | AS | PCR 1604/1605 | 16/17 |
| | | Nucleofection | | | | |
| [symbol] | HEK293T | RNP/ | crRNA 1553 | AS | PCR 1554/1555 | 37/38 |
| | | Nucleofection | | | | |

FIGS. 3 / 11 / 14

| | | | | | | |
|---|---|---|---|---|---|---|
| [symbol] | HEK293T | RNP/ | crRNA 1629 | S | PCR 2005/2006 | 33/33 |
| | | Nucleofection | | | | |
| [symbol] | HEK293T | RNP/ | crRNA 1629 | S | PCR 2005/2015 | 33/33 |
| | | Nucleofection | | | | |
| [symbol] | HEK293T | RNP/ | crRNA 1629 | S | PCR 2049/1619 | 33/33 |
| | | Nucleofection | | | | |
| [symbol] | HEK293T | RNP/ | crRNA 1629 | S | PCR 1618/1619 | 33/33 |
| | | Nucleofection | | | | |
| [symbol] | HEK293T | RNP/ | crRNA 1629 | S | PCR 2058/2059 | 237/237 |
| | | Nucleofection | | | | |
| [symbol] | HEK293T | RNP/ | crRNA 1629 | S | PCR 2049/1619 | 33/33 |
| | (GFP1-10) | Nucleofection | | | | |
| [symbol] | HEK293T | RNP/ | crRNA 1629 | S | PCR 1618/1619 | 33/33 |
| | (GFP1-10) | Nucleofection | | | | |
| [symbol] | HEK293T | RNP/ | crRNA 1629 | S | PCR 2051/2052 | 33/32 |
| | (GFP1-10) | Nucleofection | | | | |
| [symbol] | HEK293T | RNP/ | crRNA 1629 | S | PCR 2003/2004 | 33/32 |
| | (GFP1-10) | Nucleofection | | | | |
| [symbol] | HEK293T | RNP/ | crRNA 1648 | AS | PCR 1840/1841 or | 33/33 |
| | (GFP1-10) | Nucleofection | | | PCR 1652/1653 | |
| [symbol] | HEK293T | RNP/ | crRNA 1648 | AS | PCR 2008/2009 | 33/33 |
| | (GFP1-10) | Nucleofection | | | | |
| [symbol] | HEK293T | RNP/ | crRNA 1777 | S | PCR 2055/2054 | 33/34 |
| | (GFP1-10) | Nucleofection | | | | |
| [symbol] | HEK293T | RNP/ | crRNA 1777 | S | PCR 2053/2054 | 33/34 |
| | (GFP1-10) | Nucleofection | | | | |
| [symbol] | HEK293T | RNP/ | crRNA 1777 | S | PCR 2003/2004 | No homology arm, PCR |
| | (GFP1-10) | Nucleofection | | | | contatining GFP11 with |
| | | | | | | Lamin A/C homology arms |
| | | | | | | used with RAB11A crRNA |

| | | | | |
|---|---|---|---|---|
| | Repair template | | | |
| | polarity | Distance between | | |
| | (relative to | DSB and edit | Repair template | Insert size |
| | gene coding | (bp, relative to | concentration | (nucleotides/ |
| Edit | sequence) | DSB) | (μM for RNP/Nucleofection) | basepairs) |

FIG. 1

| | | | | |
|---|---|---|---|---|
| [symbol] | dsDNA circular | between −4/+2 | 3.5 nM of plasmid repair | 739 |
| | | | template and 30 ng/μl Cas9 | |
| | | | protein and 0.6 μM of | |
| | | | crRNA/TracrRNA in injection | |
| | | | buffer | |
| [symbol] | dsDNA | between −4/+2 | 6 nM or 10 nM of PCR repair | 739 |
| | | | template and 30 ng/μl Cas9 | |
| | | | protein and 0.6 μM of | |

FIGS. 2 / 10 / 11

| | | | | |
|---|---|---|---|---|
| [symbol] | dsDNA | 0 | 0.33 | 714 |
| [symbol] | dsDNA | 0 | 0.33 | 714 |
| [symbol] | dsDNA | 0 | 0.33 | 714 |
| [symbol] | dsDNA | 0 | 0.03 | 714 |
| [symbol] | dsDNA | 0 | 0.03 | 714 |
| [symbol] | dsDNA | 0 | 0.03 | 714 |
| [symbol] | dsDNA | −11 (recoded) | 0.21 | 714 |
| [symbol] | dsDNA | −11 (recoded) | 0.21 | 714 |
| [symbol] | dsDNA | −11 (recoded) | 0.21 | 714 |
| [symbol] | dsDNA | −11 (recoded) | 0.02 | 714 |
| [symbol] | dsDNA | −11 (recoded) | 0.02 | 714 |
| [symbol] | dsDNA circular | −11 (recoded) | 0.02 | 714 |
| [symbol] | dsDNA | 0 | 0.21 | 714 |
| [symbol] | dsDNA | +5 (recoded) | 0.33 | 714 |
| [symbol] | dsDNA | +5 (recoded) | 0.33 | 714 |
| [symbol] | dsDNA | +5 (recoded) | 0.33 | 714 |

FIGS. 3 / 11 / 14

| | | | | |
|---|---|---|---|---|
| [symbol] | dsDNA | 0 | 0.12 | 2229 |
| [symbol] | dsDNA | 0 | 0.12 | 1122 |
| [symbol] | dsDNA | 0 | 0.12 | 993 |
| [symbol] | dsDNA | 0 | 0.12 | 714 |
| [symbol] | dsDNA | 0 | 0.12 | 714 |
| [symbol] | dsDNA | 0 | 0.33 | 993 |
| [symbol] | dsDNA | 0 | 0.33 | 714 |
| [symbol] | dsDNA | 0 | 0.33 | 336 |
| [symbol] | dsDNA | 0 | 0.33 | 57 |
| [symbol] | dsDNA | −11 (recoded) | 0.21 | 714 |
| [symbol] | dsDNA | −11 (recoded) | 0.21 | 57 |
| [symbol] | dsDNA | −2 | 0.33 | 57 |
| [symbol] | dsDNA | −32 (recoded) | 0.33 | 57 |
| [symbol] | dsDNA | 0 | 0.33 | 57 |

| | |
|---|---|
| | Efficiency (%)(for cytometer analysis: non-nucleofected cells <0.5%)(SD: |
| Edit | Standard Deviation) |

FIG. 1

| | |
|---|---|
| [symbol] | PCR on mouse tail DNA (predicted size shift): 20.0 (5/25). 0% (0/20) of the |
| | negative for Adcy3 mCherry insertion are positive by PCR using mCherry internal |
| | primers |
| [symbol] | PCR on mouse tail DNA (predicted size shift): 31.0 (27/87, including 3 |
| | homozygous), 10 out of 10 Het clones for mCherry have perfect insertion, 11.6% |
| | (7/60) of the negative for Adcy3 mCherry insertion are positive by PCR using |
| | mCherry internal primers |

FIGS. 2 / 10 / 11

| | |
|---|---|
| [symbol] | Cytometer: 1.5 (average of independent experiments: 1.3 + 1.7)(n = 2, SD = 0.3) |
| [symbol] | Cytometer: 7.2 (average of independent experiments:4.9 + 9.4)(n = 2, SD = 3.2) |
| [symbol] | Cytometer: 14.9 (average of independent experiments: 19.2 + 14.9 + 13.2 + 15.8 + |
| | 16.3 + 12.5 + 14.9 + 12.7 + 11.8 + 18.1)(n = 10, SD = 2.5) Sequencing results can be |
| | found in FIG. S4 and S5 |
| [symbol] | Cytometer: 7.8 (average of independent experiments: 7.5 + 8)(n = 2, SD = 0.4) |
| [symbol] | Cytometer: 8.9 (average of independent experiments: 9.4 + 8.4)(n = 2, SD = 0.7) |
| [symbol] | Cytometer: 1.0 (aver, of independent experiments: 1.1 + 1.2 + 0.8) (n = 23, |
| | SD = 0.2) |
| [symbol] | Cytometer: 1.2 (average of independent experiments: 1.3 + 1.1)(n = 2, SD = 0.1) |
| [symbol] | Cytometer: 9.9 (average of independent experiments: 11.0 + 8.7)(n = 2, SD = 1.6) |
| [symbol] | Cytometer: 17.5 (average of independent experiments: 17.5 + 22.5 + 12.6)(n = 3, |
| | SD = 5.0) |
| [symbol] | Cytometer: 7.6 (average of independent experiments: 7.6 + 7.5)(n = 2, SD = 0.1) |
| [symbol] | Cytometer: 5.3 (average of independent experiments: 6.4 + 4.1)(n = 2, SD = 1.6) |
| [symbol] | Cytometer: 2.7 (average of independent experiments: 3.7 + 1.7)(n = 2, SD = 1.4) |
| [symbol] | Microscope: no Lamin A/C or RAB11A eGFP signal |
| [symbol] | Cytometer: 1.4 (average of independent experiments: 1.9 + 0.9)(n = 2, SD = 0.7) |
| [symbol] | Cytometer: 12.0 (average of independent experiments: 10.1 + 13.9)(n = 2, SD = 2.7) |
| [symbol] | Cytometer: 14.0 (average of independent experiments: 14.4 + 15.5 + 11.2 + 9.0 + |
| | 13.9 + 18l4 + 16.4 + 13.1)(n = 8, SD = 3.0) |

FIGS. 3 / 11 / 14

| | |
|---|---|
| [symbol] | Microscope: <0.1 (average of independent experiments: D(Q/1406, but few |
| | positives can be found when the entire microscope slide was examined) + <0.1 |
| | (1/1529))(n = 2, SD = 0.3) |
| [symbol] | Microscope: 0.3 (average of independent experiments: 0.5 (7/1350) + <0.1 |
| | (1/1674))(n = 2, SD = 0.3) |
| [symbol] | Microscope: 3.1 (average of independent experiments: 3.2 + 3.0)(n = 2, SD = 0.1) |
| [symbol] | Microscope: 9.8 (average of independent experiments: 11.4 + 8.1)(n = 2, SD = 2.3) |
| [symbol] | Microscope: 8.5 (average of independent experiments: 10.0 + 6.9)(n = 2, SD = 2.2) |
| [symbol] | Microscope: 17.9 (average of independent experiments: 20.4 + 15.3)(n = 2, |
| | SD = 3.6) |
| [symbol] | Microscope: 23.5 (average of independent experiments: 31.2 + 25.7 + 22.2 + |
| | 14.9)(n = 4, SD = 6.8) |
| [symbol] | Microscope: 30.5 (average of independent experiments: 29.0 + 32.0)(n = 2, |
| | SD = 2.1) |
| [symbol] | Microscope: 45.4 (average of independent experiments: 44.9 + 45.8)(n = 2, |
| | SD = 0.6) |
| [symbol] | Cytometer: 12.8 (average of independent experiments: 14.1 + 11.5)(n = 2, SD = 1.8) |
| [symbol] | Cytometer: 32.8 (average of independent experiments: 37.4 + 28.2)(n = 2, SD = 6.5) |
| [symbol] | Cytometer: 50.0 |
| [symbol] | Cytometer: 20.7 |
| [symbol] | Cytometer: 1.3 |

[symbol]

indicates data missing or illegible when filed

TABLE 2
Detailed experimental conditions and results

| | | | | | | | | |
|---|---|---|---|---|---|---|---|---|
| | | | | | | Repair | | Distance |
| | | | | | | template | Repair | between |
| | | | | crRNA/sgRNA | | left/right | template | DSN and |
| | | | | polarity | | homology | polarity | edit |
| | | | | (relative to | Repair | arms | (relative to | (bp, |
| | | | | gene coding | template | (nucleotides/ | gene coding | relative |
| Edit | Cell type | Cas9 delivery | name | sequence) | type/name | basepairs) | sequence) | to DSB) |

FIG. 4

| [symbol] | HEK293T | RNP/ | crRNA | S | ssODN 1788 | 33/0 | S | 0 |
|---|---|---|---|---|---|---|---|---|
| | (GFP1-10) | Nucleofection | 1629 | | | | | |
| [symbol] | HEK293T | RNP/ | crRNA | S | ssODN 1789 | 33/0 | AS | 0 |
| | (GFP1-10) | Nucleofection | 1629 | | | | | |
| [symbol] | HEK293T | RNP/ | crRNA | S | ssODN 1705 | 0/32 | S | 0 |
| | (GFP1-10) | Nucleofection | 1629 | | | | | |
| [symbol] | HEK293T | RNP/ | crRNA | S | ssODN 1706 | 0/32 | AS | 0 |
| | (GFP1-10) | Nucleofection | 1629 | | | | | |
| [symbol] | HEK293T | RNP/ | crRNA | AS | ssODN 1816 | 33/0 | S | +1 |
| | (GFP1-10) | Nucleofection | 1648 | | | | | |
| [symbol] | HEK293T | RNP/ | crRNA | AS | ssODN 1817 | 33/0 | AS | +1 |
| | (GFP1-10) | Nucleofection | 1648 | | | | | |
| [symbol] | HEK293T | RNP/ | crRNA | AS | ssODN 1819 | 0/33 | S | −2 |
| | (GFP1-10) | Nucleofection | 1648 | | | | | |
| [symbol] | HEK293T | RNP/ | crRNA | AS | ssODN 1820 | 0/33 | AS | −2 |
| | (GFP1-10) | Nucleofection | 1648 | | | | | |
| | | | | | | Repair | | Efficienty [%] |
| | | | | | | template | | (for cytometer |
| | | | | | | concentration | | analysis: |
| | | | | | | (μM for | Insert | non-nucleofected |
| | | | | | | RNP/ | size | cells <0.5%) |
| | | | | | | Nucleo- | (nucleotides/ | (SD: Standard |
| | | | | | Edit | fection) | basepairs) | Deviation) |
| | | | | | FIG. 4 | | | |
| | | | | | [symbol] | 5 | 126 | Microscope: 0.2 |
| | | | | | [symbol] | 5 | 126 | Microscope: 20.8 |
| | | | | | [symbol] | 5 | 126 | Microscope: 9.5 |
| | | | | | [symbol] | 5 | 126 | Microscope: 0.4 |
| | | | | | [symbol] | 5 | 126 | Cytometer: 1.6 |
| | | | | | [symbol] | 5 | 126 | Cytometer: 21.9 |
| | | | | | [symbol] | 5 | 126 | Cytometer: 15.4 |
| | | | | | [symbol] | 5 | 126 | Cytometer: 1.1 |

[symbol]

indicates data missing or illegible when filed

TABLE 3
Detailed experimental conditions and results

| | | | | | | |
|---|---|---|---|---|---|---|
| | | | | | | Repair |
| | | | | crRNA/sgRNA | | template |
| | | | | polarity | | left/right |
| | | | crRNA/ | (relative to | Repair | homology arms |
| | | | sgRNA | gene coding | template | (nucleotides/ |
| Edit | Cell type | Cas9 delivery | name | sequence) | type/name | basepairs) |

FIGS 5 / 14

| [symbol] | HEK293T | RNP/Nucleofection | crRNA 1629 | S | ssODN 1620 | 33/32 |
|---|---|---|---|---|---|---|
| | (GFP1-10) | | | | | |
| [symbol] | HEK293T | RNP/Nucleofection | crRNA 1629 | S | ssODN 1732 | 33/32 |
| | (GFP1-10) | | | | | |
| [symbol] | HEK293T | RNP/Nucleofection | crRNA 1629 | S | ssODN 1678 | 38/35 |
| | (GFP1-10) | | | | | |
| [symbol] | HEK293T | RNP/Nucleofection | crRNA 1629 | S | ssODN 1679 | 38/35 |
| | (GFP1-10) | | | | | |
| [symbol] | HEK293T | RNP/Nucleofection | crRNA 1629 | S | ssODN 1793 | 33/33 |
| | (GFP1-10) | | | | | |
| [symbol] | HEK293T | RNP/Nucleofection | crRNA 1629 | S | ssODN 1794 | 33/33 |
| | (GFP1-10) | | | | | |
| [symbol] | HEK293T | RNP/Nucleofection | crRNA 1629 | S | ssODN 1795 | 33/33 |
| | (GFP1-10) | | | | | |
| [symbol] | HEK293T | RNP/Nucleofection | crRNA 1629 | S | ssODN 1796 | 33/33 |
| | (GFP1-10) | | | | | |
| [symbol] | HEK293T | RNP/Nucleofection | crRNA 1729 | AS | ssODN 1736 | 38/33 |
| | (GFP1-10) | | | | | |
| [symbol] | HEK293T | RNP/Nucleofection | crRNA 1729 | AS | ssODN 1737 | 38/33 |
| | (GFP1-10) | | | | | |
| [symbol] | HEK293T | RNP/Nucleofection | crRNA 1728 | S | ssODN 1736 | 38/34 |
| | (GFP1-10) | | | | | |
| [symbol] | HEK293T | RNP/Nucleofection | crRNA 1728 | S | ssODN 1737 | 38/34 |
| | (GFP1-10) | | | | | |
| [symbol] | HEK293T | RNP/Nucleofection | crRNA 1648 | AS | ssODN 1778 | 33/33 |
| | (GFP1-10) | | | | | |
| [symbol] | HEK293T | RNP/Nucleofection | crRNA 1648 | AS | ssODN 1779 | 33/33 |
| | (GFP1-10) | | | | | |
| [symbol] | HEK293T | RNP/Nucleofection | crRNA 1648 | AS | ssODN 1864 | 33/33 |
| | (GFP1-10) | | | | | |
| [symbol] | HEK293T | RNP/Nucleofection | crRNA 1688 | AS | ssODN 1865 | 33/33 |
| | (GFP1-10) | | | | | |
| [symbol] | HEK293T | RNP/Nucleofection | crRNA 1648 | AS | ssODN 1831 | 33/34 |
| | (GFP1-10) | | | | | |
| [symbol] | HEK293T | RNP/Nucleofection | crRNA 1648 | AS | ssODN 1832 | 33/34 |
| | (GFP1-10) | | | | | |
| [symbol] | HEK293T | RNP/Nucleofection | crRNA 1777 | S | ssODN 1782 | 33/34 |
| | (GFP1-10) | | | | | |
| [symbol] | HEK293T | RNP/Nucleofection | crRNA 1777 | S | ssODN 1783 | 33/34 |
| | (GFP1-10) | | | | | |
| [symbol] | HEK293T | RNP/Nucleofection | crRNA 1777 | S | ssODN 1833 | 33/34 |
| | (GFP1-10) | | | | | |
| [symbol] | HEK293T | RNP/Nucleofection | crRNA 1777 | S | ssODN 1834 | 33/34 |
| | (GFP1-10) | | | | | |
| [symbol] | HEK293T | RNP/Nucleofection | crRNA 1777 | S | ssODN 1827 | 33/34 |
| | (GFP1-10) | | | | | |
| [symbol] | HEK293T | RNP/Nucleofection | crRNA 1777 | S | ssODN 1828 | 33/34 |
| | (GFP1-10) | | | | | |
| [symbol] | HEK293T | RNP/Nucleofection | crRNA 1776 | AS | ssODN 1782 | 33/34 |
| | (GFP1-10) | | | | | |
| [symbol] | HEK293T | RNP/Nucleofection | crRNA 1776 | AS | ssODN 1783 | 33/34 |
| | (GFP1-10) | | | | | |
| [symbol] | HEK293T | RNP/Nucleofection | crRNA 1776 | AS | ssODN 1833 | 33/34 |
| | (GFP1-10) | | | | | |
| [symbol] | HEK293T | RNP/Nucleofection | crRNA 1776 | AS | ssODN 1834 | 33/34 |
| | (GFP1-10) | | | | | |
| [symbol] | HEK293T | RNP/Nucleofection | crRNA 1776 | AS | ssODN 1827 | 33/34 |
| | (GFP1-10) | | | | | |
| [symbol] | HEK293T | RNP/Nucleofection | crRNA 1776 | AS | ssODN 1828 | 33/34 |
| | (GFP1-10) | | | | | |
| [symbol] | HEK293T | RNP/Nucleofection | crRNA 1910 | AS | ssODN 1911 | 35/32 |
| | (GFP1-10) | | | | | |
| [symbol] | HEK293T | RNP/Nucleofection | crRNA 1910 | AS | ssODN 1912 | 35/32 |
| | (GFP1-10) | | | | | |
| [symbol] | HEK293T | RNP/Nucleofection | crRNA 1910 | AS | ssODN 1924 | 35/35 |
| | (GFP1-10) | | | | | |
| [symbol] | HEK293T | RNP/Nucleofection | crRNA 1910 | AS | ssODN 1925 | 35/35 |
| | (GFP1-10) | | | | | |
| [symbol] | HEK293T | RNP/Nucleofection | crRNA 1909 | S | ssODN 1911 | 33/35 |
| | (GFP1-10) | | | | | |
| [symbol] | HEK293T | RNP/Nucleofection | crRNA 1909 | S | ssODN 1912 | 33/35 |
| | (GFP1-10) | | | | | |
| [symbol] | HEK293T | RNP/Nucleofection | crRNA 1909 | S | ssODN 1922 | 35/35 |
| | (GFP1-10) | | | | | |
| [symbol] | HEK293T | RNP/Nucleofection | crRNA 1909 | S | ssODN 1923 | 35/35 |
| | (GFP1-10) | | | | | |
| [symbol] | HEK293T | RNP/Nucleofection | crRNA 1748 | AS | ssODN 1751 | 34/38 |
| | (GFP1-10) | | | | | |
| [symbol] | HEK293T | RNP/Nucleofection | crRNA 1748 | AS | ssODN 1752 | 34/38 |
| | (GFP1-10) | | | | | |
| [symbol] | HEK293T | RNP/Nucleofection | crRNA 1747 | S | ssODN 1753 | 34/38 |
| | (GFP1-10) | | | | | |
| [symbol] | HEK293T | RNP/Nucleofection | crRNA 1747 | S | ssODN 1754 | 34/38 |
| | (GFP1-10) | | | | | |
| [symbol] | HEK293T | Plasmid (CAS9:: | sgPYM1 | S | ssODN 1583 | 38/40 |
| | | T2A::GFP; GFP | | | | |
| | | sorting/ | | | | |
| | | Transfection | | | | |
| [symbol] | HEK293T | Plasmid (CAS9:: | sgPYM1 | S | ssODN 1584 | 38/40 |
| | | T2A::GFP; GFP | | | | |
| | | sorting/ | | | | |
| | | Transfection | | | | |
| [symbol] | HEK293T | RNP/Nucleofection | crRNA 1729 | AS | ssODN 1734 | 33/33 |
| | (GFP1-10) | | | | | |
| [symbol] | HEK293T | RNP/Nucleofection | crRNA 1728 | S | ssODN 1734 | 32/33 |
| | (GFP1-10) | | | | | |
| | | | Distance | | | Efficiency [%] |
| | | Repair | between | | | (for cytometer |
| | | template | DSB | | Insert | analysis: non- |
| | | polarity | and edit | Repair template | size | nucleofected |
| | | (relative to | (bp, | concentration | (nucleo- | cells <0.5%) |
| | | gene coding | relative | (μM for RNP/ | tides/ | (SD: |
| | Edit | sequence | to DSB) | Nucleofection) | basepairs) | Standard Deviation |
| | FIGS. 5 /14 | | | | | |
| | [symbol] | S | 0 | 5 | 57 | Microscope: 11.6 |
| | [symbol] | AS | 0 | 5 | 57 | Microscope: 11.8 |
| | [symbol] | S | −12 (recoded) | 5 | 57 | Microscope: 17.9 |
| | [symbol] | AS | −12 (recoded) | 5 | 57 | Microscope: 11.2 |
| | [symbol] | S | +12 (recoded) | 5 | 57 | Microscope: 9.7 |
| | [symbol] | AS | +12 (recoded) | 5 | 57 | Microscope: 11.9 |
| | [symbol] | S | +33 (recoded) | 5 | 57 | Microscope: 6.3 |
| | [symbol] | AS | +33 (recoded) | 5 | 57 | Microscope: 12.4 |
| | [symbol] | S | −32 (recoded) | 5 | 57 | Microscope: 1.1 |
| | [symbol] | AS | −32 (recoded) | 5 | 57 | Microscope: 0.2 |
| | [symbol] | S | −31 (recoded) | 5 | 57 | Microscope: 12.8 |
| | [symbol] | AS | −31 (recoded) | 5 | 57 | Microscope: 1.3 |
| | [symbol] | S | −11 (recoded) | 5 | 57 | Cytometer: 14.2 |
| | [symbol] | AS | −11 (recoded) | 5 | 57 | Cytometer: 9.7 |
| | [symbol] | S | −2 | 5 | 57 | Cytometer: 25.7 |
| | [symbol] | AS | −2 | 5 | 57 | Cytometer: 32.1 |
| | [symbol] | S | +19 (recoded) | 5 | 57 | Cytometer: 2.5 |
| | [symbol] | AS | +19 (recoded) | 5 | 57 | Cytometer: 14.3 |
| | [symbol] | S | −32 (recoded) | 5 | 57 | Cytometer: 14.6 |
| | [symbol] | | | | | (experimental replicate: 14.4) |
| | [symbol] | AS | −32 (recoded) | 5 | 57 | Cytometer: 2.3 |
| | [symbol] | | | | | (experimental replicate: 1.3) |
| | [symbol] | S | −17 (recoded) | 5 | 57 | Cytometer: 31.0 |
| | [symbol] | AS | −17 (recoded) | 5 | 57 | Cytometer: 7.3 |
| | [symbol] | S | −2 | 5 | 57 | Cytometer: 36.6 |
| | [symbol] | AS | −2 | 5 | 57 | Cytometer: 31.5 |
| | [symbol] | S | −32 (recoded) | 5 | 57 | Cytometer: 10.5 |
| | [symbol] | | | | | (experimental replicate: 10.1) |
| | [symbol] | AS | −32 (recoded) | 5 | 57 | Cytometer: 1.3 |
| | [symbol] | | | | | (experimental replicate: 1.4) |
| | [symbol] | S | −17 (recoded) | 5 | 57 | Cytometer: 21.6 |
| | [symbol] | AS | −17 (recoded) | 5 | 57 | Cytometer: 3.9 |
| | [symbol] | S | −2 | 5 | 57 | Cytometer: 22.6 |
| | [symbol] | AS | −2 | 5 | 57 | Cytometer: 24.6 |
| | [symbol] | S | −3 | 5 | 57 | Cytometer: 26.5 |
| | [symbol] | AS | −3 | 5 | 57 | Cytometer: 31.5 |
| | [symbol] | S | +21 (recoded) | 5 | 57 | Cytometer: 1.0 |
| | [symbol] | AS | +21 (recoded) | 5 | 57 | Cytometer: 5.5 |
| | [symbol] | S | +2 | 5 | 57 | Cytometer: 15.5 |
| | [symbol] | AS | +2 | 5 | 57 | Cytometer: 18.8 |
| | [symbol] | S | +26 (recoded) | 5 | 57 | Cytometer: 0.7 |
| | [symbol] | AS | +26 (recoded) | 5 | 57 | Cytometer: 3.4 |
| | [symbol] | S | +29 (recoded) | 5 | 57 | Microscope: 1.8 |
| | [symbol] | AS | +29 (recoded) | 5 | 57 | Microscope: 11.0 |
| | [symbol] | S | +41 (recoded) | 5 | 57 | Microscope: 0.4 |
| | [symbol] | AS | +41 (recoded) | 5 | 57 | Microscope: 10.3 |
| | [symbol] | S | +1 and +25 | 3.24 pmol of | 6 and 66 | PCR single cell colonies |
| | | | (recoded) | ssODN and 892 ng | | (predicted size shift and |
| | | | | of CAS9 plasmid | | Restriction Enzyme digest): |
| | | | | in a mix of 10.8 | | 3.7 (2/58) for RE and 3x Flag |
| | | | | μl containing | | insertion, 0 (0/58) for 3x Flag |
| | | | | 3.6 μl of X- | | insertion alone, 0 (0/58) for |
| | | | | tremeGENE 9 | | RE insertion alone |
| | [symbol] | AS | +1 and +25 | 3.24 pmol of | 6 and 66 | PCR single cell colonies |
| | | | (recoded) | ssODN and 892 ng | | (predicted size shift and |
| | | | | of CAS9 plasmid | | Restriction Enzyme digest): |
| | | | | in a mix of 10.8 | | 44.6 (21/47) including 7 |
| | | | | μl containing | | homozygous) for RE and 3x Flag |
| | | | | 3.6 μl of X- | | insertion, 0 (0/47) for 3x Flag |
| | | | | tremeGENE 9 | | insertion along, 10.6 (5/47) |
| | | | | | | for RE insertion alone |
| | [symbol] | S | +1 | 5 | 57 | Microscope: 2.0 |
| | [symbol] | S | +2 | 5 | 57 | Microscope: 22.8 |

[symbol]

indicates data missing or illegible when filed

TABLE 4
Detailed experimental conditions and results

| | | | | | | |
|---|---|---|---|---|---|---|
| | | | | crRNA/ | | |
| | | | | sgRNA | | |
| | | | | polarity | | |
| | | | | relative | | |
| | | | | to | | |
| | | | crRNA/ | gene | | Repair template left/right |
| | | | sgRNA | coding | Repair template | homology arms |
| Edit | Cell type | Cas9 delivery | name | sequence) | type/name | (nucleotides/basepairs) |

FIGS. 5 / 6 / 14

| | | | | | | |
|---|---|---|---|---|---|---|
| [symbol] | HEK293T | Plasmid (CAS9::T2A:: | sgPYM1 | S | ssODN 1582 | 38/37 |
| | | GFP; GFP sorting)/ | | | | |
| | | Transfection | | | | |
| [symbol] | HEK293T | Plasmid (CAS9::T2A:: | sgPYM1 | S | ssODN 1580 | 38/37 |
| | | GFP; GFP sorting)/ | | | | |
| | | Transfection | | | | |
| [symbol] | HEK293T | Plasmid (CAS9::T2A:: | sgPYM1 | S | ssODN 1581 | 38/37 |
| | | GFP; GFP sorting)/ | | | | |
| | | Transfection | | | | |
| [symbol] | HEK293T | Plasmid (CAS9::T2A:: | sgPYM1 | S | ssODN 1518 | 46/43 |
| | | GFP; GFP sorting)/ | | | | |
| | | Transfection | | | | |
| [symbol] | HEK293T | RNP/Nucleofection | crRNA | S | PCR 1948/1949 | 33/33 |
| | (GFP1-10) | | 1629 | | (on plasmid 1892) | |
| [symbol] | HEK293T | RNP/Nucleofection | crRNA | S | PCR 1948/1949 | 33/33 |
| | (GFP1-10) | | 1629 | | (on plasmid 1893) | |

FIGS. 7 / 15

| | | | | | | |
|---|---|---|---|---|---|---|
| [symbol] | HEK293T | RNP/Nucleofection | crRNA | AS | ssODN 1957 and | 33/33 and non-applicable |
| | (GFP1-10) | | 1648 | | ssODN 1379 | |
| | | | | | (unrelated) | |
| [symbol] | HEK293T | RNP/Nucleofection | crRNA | AS | ssODN 1955 and | no homology arm but 65/63 |
| | (GFP1-10) | | 1648 | | ssODN 1379 | homologous sequences flanking the |
| | | | | | (unrelated) | STOP (/frameshift) in GFP11 and |
| | | | | | | non-applicable |
| [symbol] | HEK293T | RNP/Nucleofection | crRNA | AS | ssODN 1957 and | 33/33 and no homology arm but |
| | (GFP1-10) | | 1648 | | ssODN 1955 | 65/63 homologous sequences |
| | | | | | | flanking the STOP/frameshift |
| [symbol] | HEK293T | RNP/Nucleofection | crRNA | AS | ssODN 1954 and | 33/33 and non-applicable |
| | (GFP1-10) | | 1648 | | ssODN 1379 | |
| | | | | | (unrelated) | |
| [symbol] | HEK293T | RNP/Nucleofection | crRNA | AS | ssODN 1956 and | 33/33 and non-applicable |
| | (GFP1-10) | | 1648 | | ssODN 1379 | |
| | | | | | (unrelated) | |
| [symbol] | HEK293T | RNP/Nucleofection | crRNA | AS | ssODN 1956 and | 33/33 and no homology arm but |
| | (GFP1-10) | | 1648 | | ssODN 1955 | 65/63 homologous sequences |
| | | | | | | flanking the STOP |
| [symbol] | HEK293T | RNP/Nucleofection | crRNA | AS | PCR 2083/2084 | 33/33 and non-applicable |
| | (GFP1-10) | | 1648 | | (on ssODN 1957) + | |
| | | | | | PCR 2090/2091 | |
| | | | | | (on ssODN 1379, | |
| | | | | | unrelated) | |
| [symbol] | HEK293T | RNP/Nucleofection | crRNA | AS | PCR 2086/2087 | no homology arm but 65/63 |
| | (GFP1-10) | | 1648 | | (on ssODN 1955) + | homologous sequences flanking the |
| | | | | | PCR 2090/2091 | STOP (/frameshift) in GFP11 and |
| | | | | | (on ssODN 1379, | non-applicable |
| | | | | | unrelated) | |
| [symbol] | HEK293T | RNP/Nucleofection | crRNA | AS | PCR 2083/2084 | 33/33 and no homology arm but |
| | (GFP1-10) | | 1648 | | (on ssODN 1957) + | 65/63 homologous sequences |
| | | | | | PCR 2086/2087 | flanking the STOP/frameshift |
| | | | | | (on ssODN 1955) | |
| [symbol] | HEK293T | RNP/Nucleofection | crRNA | AS | PCR 2083/2084 | 33/33 and non-applicable |
| | (GFP1-10) | | 1648 | | (on ssODN 1954) + | |
| | | | | | PCR 2090/2091 | |
| | | | | | (on ssODN 1379, | |
| | | | | | unrelated) | |
| [symbol] | HEK293T | RNP/Nucleofection | crRNA | S | ssODN 1799 | 33/32 |
| | (GFP1-10) | | 1629 | | | |
| [symbol] | HEK293T | RNP/Nucleofection | crRNA | S | ssODN 1835 | 33/32 |
| | (GFP1-10) | | 1629 | | | |
| [symbol] | HEK293T | RNP/Nucleofection | crRNA | S | ssODN 1799 and | 33/32 and ssODN without homology |
| | (GFP1-10) | | 1629 | | ssODN 1813 | arm and no mutations |
| [symbol] | HEK293T | RNP/Nucleofection | crRNA | S | ssODN 1799 and | 33/32 and ssODN without homology |
| | (GFP1-10) | | 1629 | | ssODN 1804 | arm and 1 mutation every 3 nt |
| [symbol] | HEK293T | RNP/Nucleofection | crRNA | S | ssODN 1799 and | 33/32 and ssODN without homology |
| | (GFP1-10) | | 1629 | | ssODN 1805 | arm and 1 mutation every 6 nt |
| [symbol] | HEK293T | RNP/Nucleofection | crRNA | S | ssODN 1799 and | 33/32 and ssODN without homology |
| | (GFP1-10) | | 1629 | | ssODN 1806 | arm and 1 mutation every 12 nt |
| [symbol] | HEK293T | RNP/Nucleofection | crRNA | AS | ssODN 1799 | No homology arm, ssODN containing |
| | (GFP1-10) | | 1648 | | | extra-sequence::GFP11-Myc without |
| | | | | | | mutation::extra-sequence with |
| | | | | | | Lamin A/C homology arms used with |
| | | | | | | RAB11A crRNA |

| | | | | |
|---|---|---|---|---|
| | | Distance | | |
| | Repair | between | | |
| | template | DSB and | | |
| | polarity | edit (bp, | | |
| | (relative to | relative | | Insert size |
| | gene coding | to | Repair template concentration | (nucleotides/ |
| Edit | sequence) | DSB) | (μM for RNP/Nucleofection) | basepairs) |

FIGS. 5 / 6 /14

| | | | | |
|---|---|---|---|---|
| [symbol] | S | +1 and −23 | 3.24 pmol of ssODN and 892 ng of | 6 and 66 |
| | | | Cas9 plasmid in a mix of 10.8 μl | |
| | | | containing 3.6 μl of X-tremeGENE | |
| | | | 9 | |
| [symbol] | S | +1 and −23 | 3.24 pmol of ssODN and 892 ng of | 6 and 66 |
| | | (recoded) | Cas9 plasmid in a mix of 10.8 μl | |
| | | | containing 3.6 μl of X-tremeGENE | |
| | | | 9 | |
| [symbol] | AS | +1 and −23 | 3.24 pmol of ssODN and 892 ng of | 6 and 66 |
| | | (recoded) | Cas9 plasmid in a mix of 10.8 μl | |
| | | | containing 3.6 μl of X-tremeGENE | |
| | | | 9 | |
| [symbol] | S | +1 | 3.24 pmol of ssODN and 892 ng of | 66 |
| | | | Cas9 plasmid in a mix of 10.8 μl | |
| | | | containing 3.6 μl of X-tremeGENE | |
| | | | 9 | |
| [symbol] | dsDNA | 0 and +33 | 0.23 | 57 and 708 |
| [symbol] | dsDNA | 0 and +33 | 0.23 | 57 and 708 |
| | | (recoded) | | |

FIGS. 7 / 15

| | | | | |
|---|---|---|---|---|
| [symbol] | S and non- | 0 | 5 and 5 | 133 |
| | applicable | | | |
| [symbol] | S and non- | 0 | 5 and 5 | 129 |
| | applicable | | | |
| [symbol] | S and S | 0 | 5 and 5 | 133 and 129 |
| [symbol] | S and non- | 0 | 5 and 5 | 132 |
| | applicable | | | |
| [symbol] | S and non- | 0 | 5 and 5 | 132 |
| | applicable | | | |
| [symbol] | S and S | 0 | 5 and 5 | 133 and 129 |
| [symbol] | dsDNA and | 0 | 0.33 and 0.33 | 133 |
| | dsDNA | | | |
| [symbol] | dsDNA and | 0 | 0.33 and 0.33 | 129 |
| | dsDNA | | | |
| [symbol] | dsDNA and | 0 | 0.33 and 0.33 | 133 and 129 |
| | dsDNA | | | |
| [symbol] | dsDNA and | 0 | 0.33 and 0.33 | 132 |
| | dsDNA | | | |
| [symbol] | S | 0 | 5 | 132 |
| [symbol] | S | 0 | 5 | 132 |
| [symbol] | S | 0 | 5 and 5 | 132 |
| [symbol] | S | 0 | 5 and 5 | 132 |
| [symbol] | S | 0 | 5 and 5 | 132 |
| [symbol] | S | 0 | 5 and 5 | 132 |
| [symbol] | S | 0 | 5 | 132 |

| | |
|---|---|
| | Efficiency (%) (for cytometer analysis: non-nucleofected cells <0.5%)(SD: |
| Edit | Standard Deviation) |

FIGS. 5 / 6 / 14

| | |
|---|---|
| [symbol] | PCR single cell colonies (predicted size shift and Restriction Enzyme digest): 12.6 |
| | (8/63) for RE and 3xFlag insertion, 0 (0/63) for 3xFlag insertion alone, 46.0 (29/63) |
| | for RE insertion alone. Total edits: 58.7 (37/63) |
| [symbol] | PCR single cell colonies (predicted size shift and Restriction Enzyme digest): 25.8 |
| | (15/58, including 3 homozygous) for RE and 3xFlag insertion, 1.7 (1/58) for 3xFlag |
| | insertion alone, 10.3 (6/58) for RE insertion alone. Total edits: 37.9 (22/58) |
| [symbol] | PCR single cell colonies (predicted size shift and Restriction Enzyme digest): 3.2 |
| | (2/61) for RE and 3xFlag insertion, 0 (0/61) for 3xFlag insertion alone, 0 (0/61) for |
| | RE insertion alone. Total edits: 3.2 (2/61) |
| [symbol] | PCR single cell colonies (predicted size shift): 53.5 (30/56, including 7 |
| | homozygous) |
| [symbol] | Microscope: 8.6 (160/1842) for GFP11 and tagRFP insertion, 0.7 (13/1842) for |
| | tagRFP insertion alone, 10.8 (199/1842) for GFP11 insertion alone. Total edits: |
| | 20.1 (372/1842) |
| [symbol] | Microscope: 17.6 (288/1629) for GFP11 and tagRFP insertion, 0.1 (3/1629) for |
| | tagRFP insertion alone, 4.4 (73/1629) for GFP11 insertion alone. Total edits: 22.3 |
| | (364/1629) |

FIGS. 7 / 15

| | |
|---|---|
| [symbol] | Cytometer: 0.3 (average of independent experiments: 0.3 + 0.3)(n = 2, SD = 0) |
| [symbol] | Cytometer: 0.4 (average of independent experiments: 0.6 + 0.2)(n = 2, SD = 0.3) |
| [symbol] | Cytometer: 3.2 (average of independent experiments: 4.0 + 2.4)(n = 2, SD = 1.1) |
| | (GFP positive cells confirmed by microscopy) |
| [symbol] | Cytometer: 17.2 (average of independent experiments: 19.6 + 14.7)(n = 2, SD = 3.5) |
| [symbol] | Cytometer: 0.6 |
| [symbol] | Cytometer: 4.1 |
| [symbol] | Cytometer: 0.5 (average of independent experiments: 0.4 + 0.5)(n = 2, SD = 0.1) |
| [symbol] | Cytometer: 0.6 (average of independent experiments: 0.5 + 0.7)(n = 2, SD = 0.1) |
| [symbol] | Cytometer: 2.3 (average of independent experiments: 2.2 + 2.3)(n = 2, SD = 0.1) |
| | (GFP positive cells confirmed by microscopy) |
| [symbol] | Cytometer: 21.7 (average of independent experiments: 19.7 + 23.6)(n = 2, SD = 2.8) |
| [symbol] | DNA was extracted from cells edited with ssODN 1799 or ssODN 1835. Next the |
| [symbol] | DNA from these experiments was mixed and the insertion was PCR amplified and |
| | sequenced by Illumina technology (Barcode 10). This control was performed to |
| | ensure that template switching does not occur during PCR amplication (PCR control |
| | in FIG. 15 and Table 15) |
| [symbol] | PCR amplicon sequencing using Illumina technology (Barcode 5, No mutation). |
| | See FIG. 15 and Table 15 |
| [symbol] | PCR amplicon sequencing using Illumina technology (Barcode 6, 1/3 mutations). |
| | See FIG. 15 and Table 15 |
| [symbol] | PCR amplicon sequencing using Illumina technology (Barcode 7, 1/6 mutations). |
| | See FIG. 15 and Table 15 |
| [symbol] | PCR amplicon sequencing using Illumina technology (Barcode 8, 1/12 mutations). |
| | See FIG. 15 and Table 15 |
| [symbol] | No PCR positive signal was detected (Barcode 9) |

[symbol]

indicates data missing or illegible when filed

TABLE 5
Repair templates used in this study

| | crRNA/ | Repair | |
|---|---|---|---|
| Gene | sgRNA | template | Homology arms |
| targeted | used | coding for | (nucleotides/basepairs) |
| non- | non- | unrelated | non-applicable |
| applicable | applicable | ssODN | |
| | | (142 nt) | |
| non- | non- | unrelated | non-applicable |
| applicable | applicable | PCR donor | |
| | | (142 nt) | |
| [symbol] | 1629 | [symbol] | 33/32 |
| [symbol] | 1629 | [symbol] | 33/32 |
| [symbol] | 1629 | [symbol] | 33/32 |
| [symbol] | 1629 | [symbol] | 0/0 |
| [symbol] | 1629 | [symbol] | 16/16 |
| [symbol] | 1629 | [symbol] | 33/33 |
| [symbol] | 1629 | [symbol] | 237/237 |
| [symbol] | 1629 | [symbol] | 518/518 |
| [symbol] | 1629 | [symbol] | 518/518 |
| [symbol] | 1629 | [symbol] | 33/32 |
| [symbol] | 1629 | [symbol] | 33/33 |
| [symbol] | 1629 | [symbol] | 33/33 |
| [symbol] | 1629 | [symbol] | 33/33 |
| [symbol] | 1629 | [symbol] | 33/0 |
| [symbol] | 1629 | [symbol] | 33/0 |
| [symbol] | 1629 | [symbol] | 0/32 |
| [symbol] | 1629 | [symbol] | 0/32 |
| [symbol] | 1629 | [symbol] | 33/33 |
| [symbol] | 1629 | [symbol] | 33/33 |
| [symbol] | 1629 | [symbol] | 33/32 |
| [symbol] | 1629 | [symbol] | 33/32 |
| [symbol] | 1629 | [symbol] | no homology arm but homologous sequences to the |
| [symbol] | 1629 | [symbol] | insert no homology arm but 23/24 homologous |
| | | | sequences to the insert on each side, and |
| | | | homeologous sequence (1/3 mutations) to the |
| | | | insert in the middle |
| [symbol] | 1629 | [symbol] | no homology arm but 23/24 homologous sequences |
| | | | to the insert on each side, and homeologous |
| | | | sequence (1/6 mutations) to the insert |
| | | | in the middle |
| [symbol] | 1629 | [symbol] | no homology arm but 23/24 homologous sequences |
| | | | to the insert on each side, and homeologous |
| | | | sequence (1/12 mutations) to the insert |
| | | | in the middle |
| Gene | Distance | | Polarity (S = Sense; |
| targeted | from DSB | Type and Name | AS = AntiSense) |
| non- | non- | ssODN 1379 | non-applicable |
| applicable | applicable | | |
| non- | non- | PCR 2090/2091 (on ssODN 1379) | dsDNA |
| applicable | applicable | | |
| [symbol] | 0 | ssODN 1620 | S |
| [symbol] | 0 | ssODN 1732 | AS |
| [symbol] | 0 | PCR 2003/2004 (on ssODN | dsDNA |
| | | 1620/1732) | |
| [symbol] | 0 | PCR 1630/832 (on plasmid 1698) | dsDNA |
| [symbol] | 0 | PCR 1685/1686 (on plasmid 1698) | dsDNA |
| | | or | |
| | | PCR 1858/1589 (on plasmid 1716) | |
| [symbol] | 0 | PCR 1618/1619 (on plasmid 1698) | dsDNA |
| | | or | |
| | | PCR 1743/1744 (on plasmid 1716) | |
| [symbol] | 0 | PCR 2058/2059 (on plasmid 1716) | dsDNA |
| [symbol] | 0 | PCR 1741/1742 (on plasmid 1716) | dsDNA |
| [symbol] | 0 | Plasmid 1716 | dsDNA circular |
| [symbol] | 0 | PCR 2051/2052 (on plasmid 2050) | dsDNA |
| [symbol] | 0 | PCR 2049/1619 (on plasmid 2042) | dsDNA |
| [symbol] | 0 | PCR 2005/2015 (on plasmid 1894) | dsDNA |
| [symbol] | 0 | PCR 2005/2006 (on plasmid 1894) | dsDNA |
| [symbol] | 0 | ssODN 1788 | S |
| [symbol] | 0 | ssODN 1789 | AS |
| [symbol] | 0 | ssODN 1705 | S |
| [symbol] | 0 | ssODN 1706 | AS |
| [symbol] | 0 and +33 | PCR 1948/1949 (on plasmid 1892) | dsDNA |
| [symbol] | 0 and +33 | PCR 1948/1949 (on plasmid 1893) | dsDNA |
| | (recoded) | | |
| [symbol] | 0 | ssODN 1799 | S |
| [symbol] | 0 | ssODN 1835 | S |
| [symbol] | non- | ssODN 1813 | S |
| | applicable | | |
| [symbol] | non- | ssODN 1804 | S |
| | applicable | | |
| [symbol] | non- | ssODN 1805 | S |
| | applicable | | |
| [symbol] | non- | ssODN 1806 | S |
| | applicable | | |
| | Gene | | |

| targeted | Sequence |
|---|---|
| non- | ggttcgggtggtgctccacgaggtggtatgcgcaagcacacagaatacaaaacgcgactttgtgatgcgttccgccgtg |
| applicable | aaggatactgcccgtacaacgacattgcacatatgctcacggacaagatgagctgagagttc |
| non- | as ssODN 1379 but dsDNA |
| applicable | |
| [symbol] | [symbol] |
| [symbol] | as ssODN 1620 but AS |
| [symbol] | as ssODN 1620 but dsDNA |
| [symbol] | [symbol] |
| [symbol] | [symbol] |
| [symbol] | [symbol] |
| [symbol] | [symbol] |
| [symbol] | [symbol] |
| [symbol] | see Table 8 |
| [symbol] | [symbol] |
| [symbol] | [symbol] |
| [symbol] | [symbol] |
| [symbol] | [symbol] |
| [symbol] | [symbol] |
| [symbol] | as ssODN 1788 but AS |
| [symbol] | [symbol] |
| [symbol] | as ssODN 1705 but AS |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |

[symbol]

indicates data missing or illegible when filed

TABLE 6
Repair templates used in this study

| | | | | | | Polarity | |
|---|---|---|---|---|---|---|---|
| | | | | | | (S = | |
| | | Repair | | | | Sense; | |
| | crRNA/ | template | Homology arms | | | AS = | |
| Gene | sgRNA | coding | (nucleotides/ | Distance | | Anti- | |
| targeted | used | for | basepairs) | from DSB | Type and Name | Sense) | Sequence |
| [symbol] | 1629 | [symbol] | 38/35 | −12 (recoded) | ssODN 1678 | S | [symbol] |
| [symbol] | 1629 | [symbol] | 38/35 | −12 (recoded) | ssODN 1679 | AS | as ssODN 1678 but AS |
| [symbol] | 1629 | [symbol] | 33/33 | +12 (recoded) | ssODN 1793 | S | [symbol] |
| [symbol] | 1629 | [symbol] | 33/33 | +12 (recoded) | ssODN 1794 | AS | as ssODN 1793 but AS |
| [symbol] | 1629 | [symbol] | 33/33 | +33 (recoded) | ssODN 1795 | S | [symbol] |
| [symbol] | 1629 | [symbol] | 33/33 | +33 (recoded) | ssODN 1796 | AS | as ssODN 1795 but AS |
| [symbol] | 1729 | [symbol] | 38/33 | −32 (recoded) | ssODN 1736 | S | [symbol] |
| [symbol] | 1729 | [symbol] | 38/33 | −32 (recoded) | ssODN 1737 | AS | as ssODN 1736 but AS |
| [symbol] | 1729 | [symbol] | 33/33 | +1 | ssODN 1734 | S | [symbol] |
| [symbol] | 1728 | [symbol] | 38/34 | −31 (recoded) | ssODN 1736 | S | see cr1729 −32 bp |
| | | | | | | | insertion |
| [symbol] | 1728 | [symbol] | 38/34 | −31 (recoded) | ssODN 1737 | AS | see cr1729 −32 bp |
| | | | | | | | insertion |
| [symbol] | 1728 | [symbol] | 32/33 | +2 | ssODN 1734 | S | see cr1729 −32 bp |
| | | | | | | | insertion |
| [symbol] | 1648 | [symbol] | 33/33 | −11 (recoded) | ssODN 1778 | S | [symbol] |
| [symbol] | 1648 | [symbol] | 33/33 | −11 (recoded) | ssODN 1779 | AS | as ssODN 1778 but AS |
| [symbol] | 1648 | [symbol] | 33/33 | −11(recoded) | PCR 2008/2009 | dsDNA | as ssODN 1778 but dsDNA |
| | | | | | (on ssODN | | see Lamin a/c with eGFP |
| | | | | | 1778/1779) | | PCR without homology arm |
| [symbol] | 1648 | [symbol] | 0/0 | −11 (recoded) | PCR 1630/832 | dsDNA | [symbol] |
| | | | | | (on plasmid | | |
| | | | | | 1698) | | |
| [symbol] | 1648 | [symbol] | 15/15 | −11 (recoded) | PCR 1838/1839 | dsDNA | [symbol] |
| | | | | | (on plasmid | | |
| | | | | | 1791) | | |
| [symbol] | 1648 | [symbol] | 33/33 | −11 (recoded) | PCR 1652/1653 | dsDNA | [symbol] |
| | | | | | (on plasmid | | |
| | | | | | 1698) or | | |
| | | | | | PCR 1840/1841 | | |
| | | | | | (on plasmid | | |
| | | | | | 1791) | | |
| [symbol] | 1648 | [symbol] | 63/63 | −11 (recoded) | PCR 1842/1843 | dsDNA | [symbol] |
| | | | | | (on plasmid | | |
| | | | | | 1791) | | |
| [symbol] | 1648 | [symbol] | 461/432 | −11 (recoded) | PCR 1846/1847 | dsDNA | [symbol] |
| | | | | | (on plasmid | | |
| | | | | | 1791) | | |
| [symbol] | 1648 | [symbol] | 461/432 | −11 (recoded) | Plasmid 1791 | dsDNA | see Table 9 |
| | | | | | | circular | |
| [symbol] | 1648 | [symbol] | 33/0 | +1 | ssODN 1816 | S | [symbol] |
| [symbol] | 1648 | [symbol] | 33/0 | +1 | ssODN 1817 | AS | as ssODN 1816 but AS |
| [symbol] | 1648 | [symbol] | 0/33 | −2 | ssODN 1819 | S | [symbol] |
| [symbol] | 1648 | [symbol] | 0/33 | −2 | ssoDN 1820 | AS | as ssODN 1819 but AS |
| [symbol] | 1648 | [symbol] | 33/33 | −2 | ssODN 1864 | S | [symbol] |
| [symbol] | 1648 | [symbol] | 33/33 | −2 | ssODN 1865 | AS | as ssODN 1864 but AS |
| [symbol] | 1648 | [symbol] | 33/34 | +19 (recoded) | ssODN 1831 | S | [symbol] |
| [symbol] | 1648 | [symbol] | 33/34 | +19 (recoded) | ssODN 1832 | AS | as ssODN 1831 but AS |
| [symbol] | 1648 | [symbol] | 33/33 | 0 | ssODN 1957 | S | [symbol] |
| [symbol] | 1648 | [symbol] | no homology arm but | non- | ssODN 1955 | S | [symbol] |
| | | | 65/63 homologous | applicable | | | |
| | | | sequences flanking | | | | |
| | | | the STOP (/frame- | | | | |
| | | | shift) in GFP11 | | | | |
| [symbol] | 1648 | [symbol] | 33/33 | 0 | ssODN 1956 | S | [symbol] |
| [symbol] | 1648 | [symbol] | 33/33 | 0 | ssODN 1954 | S | [symbol] |

[symbol]

indicates data missing or illegible when filed

TABLE 7
Repair templates used in this study

| | | Repair | | | | | |
|---|---|---|---|---|---|---|---|
| | crRNA/ | template | Homology arms | | | Polarity | |
| Gene | sgRNA | coding | (nudeotides/ | Distance | | (S - Sense | |
| targeted | used | for | basepairs) | from DSB | Type and Name | AS = AntiSense) | Sequence |
| [symbol] | 1648 | [symbol] | 33/33 | 0 | PCR 2083/2084 | dsDNA | [symbol] |
| | | | | | (on ssODN 1957) | | |
| [symbol] | 1648 | [symbol] | no homology arm but | non- | PCR 2086/2087 | dsDNA | [symbol] |
| | | | 65/63 homologous | applicable | (on ssODN 1955) | | |
| | | | sequences flanking | | | | |
| | | | the STOP | | | | |
| [symbol] | 1648 | [symbol] | 33/33 | 0 | PCR 2083/2084 | dsDNA | [symbol] |
| | | | | | (on ssODN 1954) | | |
| [symbol] | 1776 and 1777 | [symbol] | 33/34 | −32 (recoded) | ssODN 1782 | S | [symbol] |
| [symbol] | 1776 and 1777 | [symbol] | 33/34 | −32 (recoded) | ssODN 1783 | AS | [symbol] |
| [symbol] | 1776 and 1777 | [symbol] | 33/34 | −17 (recoded) | ssODN 1833 | S | [symbol] |
| [symbol] | 1776 and 1777 | [symbol] | 33/34 | −17 (recoded) | ssODN 1834 | AS | [symbol] |
| [symbol] | 1776 and 1777 | [symbol] | 33/34 | −2 | ssODN 1827 | S | [symbol] |
| [symbol] | 1776 and 1777 | [symbol] | 33/34 | −2 | ssODN 1828 | AS | [symbol] |
| [symbol] | 1777 | [symbol] | 33/34 | −2 | PCR 2055/2054 | dsDNA | [symbol] |
| | | | | | (on ssODN | | |
| | | | | | 1827/1828) | | |
| [symbol] | 1777 | [symbol] | 33/34 | −32 (recoded) | PCR 2053/2054 | dsDNA | [symbol] |
| | | | | | (on ssODN | | |
| | | | | | 1782/1783) | | |
| [symbol] | 1910 | [symbol] | 35/32 | −3 | ssODN 1911 | S | [symbol] |
| [symbol] | 1910 | [symbol] | 35/32 | −3 | ssODN 1912 | AS | [symbol] |
| [symbol] | 1909 | [symbol] | 33/35 | +2 | ssODN 1911 | S | [symbol] |
| [symbol] | 1909 | [symbol] | 33/35 | +2 | ssODN 1912 | AS | [symbol] |
| [symbol] | 1910 | [symbol] | 35/35 | +21 (recoded) | ssODN 1924 | S | [symbol] |
| [symbol] | 1910 | [symbol] | 35/35 | +21 (recoded) | ssODN 1925 | AS | [symbol] |
| [symbol] | 1909 | [symbol] | 35/35 | +26 (recoded) | ssODN 1922 | S | [symbol] |
| [symbol] | 1909 | [symbol] | 35/35 | +26 (recoded) | ssODN 1923 | AS | [symbol] |
| [symbol] | 1553 | [symbol] | 0/0 | +5 (recoded) | PCR 1630/832 | dsDNA | [symbol] |
| | | | | | (on plasmid | | |
| | | | | | 1698) | | |
| [symbol] | 1553 | [symbol] | 16/17 | +5 (recoded) | PCR 1604/1605 | dsDNA | [symbol] |
| | | | | | (on plasmid | | |
| | | | | | 1698) | | |
| [symbol] | 1553 | [symbol] | 37/38 | +5 (recoded) | PCR 1554/1555 | dsDNA | [symbol] |
| | | | | | (on plasmid | | |
| | | | | | 1698) | | |
| [symbol] | 1748 | [symbol] | 34/38 | +29 (recoded) | ssODN 1751 | S | [symbol] |
| [symbol] | 1748 | [symbol] | 34/38 | +29 (recoded) | ssODN 1752 | AS | [symbol] |
| [symbol] | 1747 | [symbol] | 34/38 | +41 (recoded) | ssODN 1753 | S | [symbol] |
| [symbol] | 1747 | [symbol] | 34/38 | +41 (recoded) | ssODN 1754 | AS | [symbol] |
| [symbol] | sgPYM1 | [symbol] | 38/37 | +1 and −23 | ssODN 1582 | S | [symbol] |
| [symbol] | sgPYM1 | [symbol] | 38/37 | +1 and −23 | ssODN 1580 | S | [symbol] |
| | | | | (recoded) | | | |
| [symbol] | sgPYM1 | [symbol] | 38/37 | +1 and −23 | ssODN 1581 | AS | [symbol] |
| | | | | (recoded) | | | |
| [symbol] | sgPYM1 | [symbol] | 46/43 | +1 | ssODN 1518 | S | [symbol] |
| [symbol] | sgPYM1 | [symbol] | 38/40 | +1 and +25 | ssODN 1583 | S | [symbol] |
| | | | | (recoded) | | | |
| [symbol] | sgPYM1 | [symbol] | 38/40 | +1 and +25 | ssODN 1584 | AS | [symbol] |
| | | | | (recoded) | | | |
| [symbol] | crAC3 | [symbol] | 36/36 | between −4/+2 | | dsDNA | [symbol] |
| [symbol] | crAC3 | [symbol] | 483/421 | between −4/+2 | | dsDNA circular | [symbol] |

[symbol]

indicates data missing or illegible when filed

TABLE 8
Plasmids used in this study

| Plasmid name | Backbone | Insert | Sequence |
|---|---|---|---|
| 1698 | pUC19 | [symbol] | [symbol] |
| 2050 | pUC19 | GFP11 with extra-sequence | [symbol] |
| | | [symbol] | |
| 2042 | pUC19 | eGFP with extra-sequence | [symbol] |
| | | [symbol] | |
| 1894 | pUC19 | eGFP with extra-sequence and tagRFP | [symbol] |
| | | [symbol] | |
| 1716 | pUC19 | [symbol] | [symbol] |

[symbol]

indicates data missing or illegible when filed

TABLE 9
Plasmids used in this study

| Plasmid name | Backbone | Insert | Sequence |
|---|---|---|---|
| 1791 | pUC19 | [symbol] | [symbol] |
| 1892 | pUC19 | [symbol] | [symbol] |
| 1893 | pUC19 | [symbol] | [symbol] |
| sgPYM1 | pX458 | sgRNA for PYM1 | see Table 14 |
| pBS-AC3CtermGenomic- | pBlueScript-KS | [symbol] | [symbol] |
| mCherry | | | |

[symbol]

indicates data missing or illegible when filed

TABLE 10
Primers used in this study

| Primers name | F/R | Description | Sequence (5′ to 3′) |
|---|---|---|---|
| 2090 | F | amplification of ssODN 1379 | ggttcgggtggtgctccac |
| 2091 | R | amplification of ssODN 1379 | gaactctcagctcatcttg |
| 832 | R | amplification of [symbol] without homology arm | [symbol] |
| 1630 | F | amplification of [symbol] without homology arm | [symbol] |
| 1676 | F | genotyping and sequencing of [symbol] insert | [symbol] |
| 1677 | R | genotyping and sequencing of [symbol] insert | [symbol] |
| 2014 | R | genotyping and sequencing of [symbol] insert | [symbol] |
| 2044 | R | genotyping and sequencing of [symbol] insert | [symbol] |
| 2045 | F | genotyping and sequencing of [symbol] insert | [symbol] |
| 2047 | F | genotyping and sequencing of [symbol] insert | [symbol] |
| 3001 | F | genotyping and sequencing of [symbol] insert | [symbol] |
| 1762 / HK13 | F | genotyping and sequencing of mCherry insert | [symbol] |
| 1763 / HK14 | R | genotyping and sequencing of mCherry insert | [symbol] |
| 1766 | R | genotyping and sequencing of mCherry insert | [symbol] |
| 1767 | F | genotyping and sequencing of mCherry insert | [symbol] |
| 1768 | R | genotyping and sequencing of mCherry insert | [symbol] |
| 1769 | F | genotyping and sequencing of mCherry insert | [symbol] |
| 1773 | F | genotyping and sequencing of mCherry insert | [symbol] |
| 1685 | F | amplification of Lamin A/C [symbol] repair template | [symbol] |
| | | (cr1629) with ~15 bp homology arm | |
| 1686 | R | amplification of Lamin A/C [symbol] repair template (cr1629) | [symbol] |
| | | (cr1629) with ~15 bp homology arm | |
| 1858 | F | amplification of Lamin A/C [symbol] repair template (cr1629) | [symbol] |
| | | (cr1629) with ~15 bp homology arm | |
| 1859 | R | amplification of Lamin A/C [symbol] repair template (cr1629) | [symbol] |
| | | (cr1629) with ~15 bp homology arm | |
| 1618 | F | amplification of Lamin A/C [symbol] repair template (cr1629) | [symbol] |
| | | with ~35 bp homology arm | |
| 1619 | R | amplification of Lamin A/C [symbol] repair template (cr1629) | [symbol] |
| | | with ~35 bp homology arm | |
| 1743 | F | amplification of Lamin A/C [symbol] repair template (cr1629) | [symbol] |
| | | with ~35 bp homology arm | |

[symbol]

indicates data missing or illegible when filed

TABLE 11
Primers used in this study

| Primers | | | |
|---|---|---|---|
| name | F/R | Description | Sequence (5′ to 3′) |
| 1744 | R | amplification of Lamin A/C eGFP repair template (cr1629) | [symbol] |
| | | with ~35 bp homology arm | |
| 2003 | F | amplification of Lamin A/C GFP11 repair template (cr1629) | [symbol] |
| | | with ~35 bp homology arm | |
| 2004 | R | amplification of Lamin A/C GFP11 repair template (cr1629) | [symbol] |
| | | with ~35 bp homology arm | |
| 2058 | F | amplification of Lamin A/C eGFP repair template (cr1629) | [symbol] |
| | | with ~240 bp homology arm | |
| 2059 | R | amplification of Lamin A/C eGFP repair template (cr1629) | [symbol] |
| | | with ~240 bp homology arm | |
| 1741 | F | amplification of Lamin A/C eGFP repair template (cr1629) | [symbol] |
| | | with ~500 bp homology arm | |
| 1742 | R | amplification of Lamin A/C eGFP repair template (cr1629) | [symbol] |
| | | with ~500 bp homology arm | |
| 2051 | F | amplification of Lamin A/C GFP11::extra-sequence (336 bp insert) | [symbol] |
| | | repair template (cr1629) with ~35 bp homology arm | |
| 2052 | R | amplification of Lamin A/C GFP11::extra-sequence (336 bp insert) | [symbol] |
| | | repair template (cr1629) with ~35 bp homology arm | |
| 2049 | F | amplification of Lamin A/C extra-sequence::eGFP (993 bp insert) | [symbol] |
| | | repair template (cr1629) with ~35 bp homology arm | |
| 2015 | R | amplification of Lamin A/C TEV::eGFP::extra-sequence (1112 bp insert) | [symbol] |
| | | repair template (cr1629) with ~35 bp homology arm | |
| 2005 | F | amplification of Lamin A/C TEV::eGFP::extra-sequence::tagRFP (2229 bp | [symbol] |
| | | insert)(cr1629) with ~35 bp homology arm | |
| 2006 | R | amplification of Lamin A/C TEV::eGFP::extra-sequence::tagRFP (2229 bp | [symbol] |
| | | insert)(cr1629) with with ~35 bp homology arm | |
| 1948 | F | amplification of Lamin A/C GFP11 at cut and tagRFP 33 bp downstream | [symbol] |
| | | (cr1629) with ~35 bp homology arm | |
| 1949 | R | amplification of Lamin A/C GFP11 at cut and tagRFP 33 bp downstream | [symbol] |
| | | (cr1629) with ~35 bp homology arm | |
| 1965 | R | Lamin A/C Illumina sequencing with barcode 5 | caagcagaagacggcatacgagatacagcagtgactggagttcagacgtgtgctcttccgatc [symbol] |
| 1966 | R | Lamin A/C Illumina sequencing with barcode 6 | caagcagaagacggcatacgagatcacgtgtgactggagttcagacgtgtgctcttccgatc [symbol] |
| 1967 | R | Lamin A/C Illumina sequencing with barcode 7 | caagcagaagacggcatacgagatgtgatggtgactggagttcagacgtgtctcttccgatc [symbol] |
| 1968 | R | Lamin A/C Illumina sequencing with barcode 8 | caagcagaagacggcatacgagattgttacgtgactggagttcagacgtgtgctcttccgatc [symbol] |
| 1969 | R | Lamin A/C Illumina sequencing with barcode 9 | caagcagaagacggcatacgagatagatccgtgactggagttcagacgtgtgctcttccgatc [symbol] |
| 1970 | R | Lamin A/C Illumina sequencing with barcode 10 | caagcagaagacggcatacgagatcccggagtgactggagttcagacgtgtgctcttccgatc [symbol] |
| 390 | F | pre-amplification of the edit for Illumina sequencing (in insert) | [symbol] |
| 1849 | R | pre-amplification of the edit for Illumina sequencing (in Lamin A/C | [symbol] |
| 1928 | F | illumina sequencing (in insert) | aatgatacggcgaccaccgagatctacactctttccctacacgacgctcttccgatct [symbol] |

[symbol]

indicates data missing or illegible when filed

TABLE 12
Primers used in this study

| Primers name | F/R | Description | Sequence (5′ to 3′) |
|---|---|---|---|
| 1712 | F | Lamin A/C genotyping and sequencing | gaaggtctgaggcaatgggg |
| 1713 | R | Lamin A/C genotyping and sequencing | gatgtagaccgccaagcgat |
| 2076 | F | Lamin A/C genotyping and sequencing | ggccggcgcactccgactc |
| 2077 | R | Lamin A/C genotyping and sequencing | caagcgatcattgagctcc |
| 1838 | F | amplification of RAB11A [symbol] repair template (cr1648) with | ctcggccgcgcaatg [symbol] |
| | | ~15 bp homology arm | |
| 1839 | R | amplification of RAB11A eGFP repair template (cr1648) with | tagtcgtactcgtcgtcac |
| | | ~15 bp homology arm | |
| 1652 | F | amplification of RAB11A [symbol] repair template (cr1648) with | gctcccgccctttcgctctc |
| | | ~35 bp homology arm | ggccgcgcaatg [symbol] |
| 1653 | R | amplification of RAB11A [symbol] repair template (cr1648) with | gcctcacctttaaagaggtagtc |
| | | ~35 bp homology arm | gtactcgtcgtcacgtgttcc [symbol] |
| 1840 | F | amplification of RAB11A eGFP repair template (cr1648) with | gctcccgcccttcgctc |
| | | ~35 bp homology arm | |
| 1841 | R | amplification of RAB11A eGFP repair template (cr1648) with | gcctcacctttaaagagg |
| | | ~35 bp homology arm | |
| 2008 | F | amplification of RAB11A GFP11 repair template (cr1648) with | gctccagccctttcgctcc |
| | | ~35 bp homology arm | |
| 2009 | R | amplification of RAB11A GFP11 repair template (cr1648) with | gcctcacctttaacgaggtag |
| | | ~35 bp homology arm | |
| 1846 | F | amplification of RAB11A eGFP repair template (cr1648) with | ggaaccgccacgcatgtg |
| | | ~50 bp homology arm | |
| 1847 | R | amplification of RAB11A eGFP repair template (cr1648) with | cagaggggcttcgggagag |
| | | ~50 bp homology arm | |
| 2053 | F | amplification of RAB11A GFP11 repair template (cr1777, | gctcccgccctttcgctcc |
| | | at −32 bp from DSB) with ~35 bp homology arm | |
| 2055 | F | amplification of RAB11A GFP11 repair template (cr1777, | atgggctcccgcgacgacg |
| | | at −2 bp from DSB) with ~35 bp homology arm | |
| 2054 | R | amplification of RAB11A GFP11 repair template (cr1777) | gtgtagagtgcgagagcc |
| | | with ~35 bp homology arm | |
| 2083 | F | amplification of RAB11A extra-sequence::GFP11(with/without | ttcgctcctcggctgcgc |
| | | STOP/frameshift)::extra-sequence repair template (cr1648) | |
| | | with ~35 bp homology arm | |
| 2084 | R | amplification of RAB11A extra-sequence::GFP11(with/without | gcctcaccttcaaagagg |
| | | STOP/frameshift)::extra-sequence repair template (cr1648) | |
| | | with ~35 bp homology arm | |
| 2086 | F | amplification of extra-sequence::GFP11(without STOP/ | aaagatcatgatatcgattac |
| | | frameshift)::extra-sequence repair template | |
| 2087 | R | amplification of extra-sequence::GFP11(without STOP/ | cagatcctctcctgatatcag |
| | | frameshift)::extra-sequence repair template | |
| 1604 | F | amplification of SMC3 [symbol] repair template (cr1553) with | gaagatgataccacacacgga [symbol] |
| | | ~15 bp homology arm | |
| 1605 | R | amplification of SMC3 [symbol] repair template (cr1553) with | gtagtattccccaatta [symbol] |
| | | ~15 bp homology arm | |
| 1554 | F | amplification of SMC3 [symbol] repair template (cr1553) with | gagatggccaaagactttgtaga |
| | | ~35 bp homology arm | agtgataccacacacgga [symbol] |

[symbol]

indicates data missing or illegible when filed

TABLE 13
Primers used in this study

| Primers name | F/R | Description | Sequence (5′ to 3′) |
|---|---|---|---|
| 1555 | R | amplification of SMC3 [symbol] repair template (cr1553) | [symbol] |
| | | with ~35 bp homology arm | |
| 1483 | F | PYM1 genotyping | tgtacggtgtattggcactcg |
| 1485 | R | PYM1 genotyping | gatagttgcccctccttcca |
| sgPYM1 cloning | F | PYM1 sgRNA cloning | caccgcgtcaacacagcgacctga |
| sgPYM1 cloning | R | PYM1 sgRNA cloning | aaactcaggtcgctgtgttgacgc |
| 1596 | F | amplification of mouse Adcy3 mCherry repair | ctctgttacactgcccac |
| | | template (crAdcy3) with ~35 bp homology arm | |
| 1597 | R | amplification of mouse Adcy3 mCherry repair | ccccttcctatgtggacc |
| | | template (crAdcy3) with ~35 bp homology arm | |
| 1760 / HK15 | F | mouse Adcy3 genotyping and sequencing | ccttcgagagtacggcttcc |
| 1761 / HK16 | R | mouse Adcy3 genotyping and sequencing | ggaacaccaggacttggtca |
| 1772 | F | mouse Adcy3 genotyping and sequencing | ttgtgaggcgaggtcccatc |
| 1764/ HK11 | F | mouse Adcy3 genotyping and sequencing | gacatccggggcaatacggtc |
| HK12 | R | mouse Adcy3 genotyping and sequencing | gactgtagcaagagctcagaaga |
| 1765 | R | mouse Adcy3 genotyping and sequencing | gctcagaagacaaggcaatattg |

[symbol]

indicates data missing or illegible when filed

TABLE 14
crRNA/sgRNA used in this study

| Guide | | | | |
|---|---|---|---|---|
| Name | Target | Type | Polarity | Sequence |
| 1629 | Lamin A/C | crRNA | S | ccatggagaccccgtcccag |
| 1648 | RAB11A | crRNA | AS | ggtagtcgtactcgtcgtcg |
| 1728 | Lamin A/C | crRNA | S | gcggcgcgccacccgcagcg |
| 1729 | Lamin A/C | crRNA | AS | agctggcctgcgccccgctg |
| 1776 | RAB11A | crRNA | AS | ccatggcctcacctttaaag |
| 1777 | RAB11A | crRNA | S | gagtacgactacctctttaa |
| 1909 | RAB11A | crRNA | S | aaccactgaaaacaagccaa |
| 1910 | RAB11A | crRNA | AS | ttctgacagcactgcacctt |
| 1553 | SMC3 | crRNA | AS | attttccaattaaccatgtg |
| 1747 | SMC3 | crRNA | S | tgatgtgatcacagcagaga |
| 1748 | SMC3 | crRNA | AS | atcatcttctacaaagtctt |
| sgPYM1 | PYM1 | sgRNA | S | gcgtcaacacagcgacctga |
| crAdcy3 | mouse | crRNA | AS | gtggagccagaggtcgctca |
| | Adcy3 | | | |

TABLE 15
Classification of reads from the Illumina sequencing experiment

| | | | | Unexpected | | Reads with |
|---|---|---|---|---|---|---|
| | Total | | Below | mutation at | Used in | switching |
| | sequencing | Do not fully | quality | diagnostic | downstream | detected (% of the |
| Sample | reads | map to template | threshold | position | analysis | previous column) |
| No mutation | 3,369,768 | 21.60% | 42.30% | 0.20% | 36.00% | 0.00% |
| PCR control | 3,241,689 | 20.10% | 56.40% | 0.20% | 23.50% | 0.02% |
| 1/3 | 3,411,796 | 20.80% | 42.30% | 0.40% | 36.60% | 0.02% |
| 1/6 | 5,680,820 | 21.20% | 42.40% | 0.20% | 36.20% | 0.50% |
| 1/12 | 6,414,459 | 21.00% | 42.40% | 0.10% | 36.50% | 1.40% |

1. A double-stranded, linear donor polynucleotide comprising a polynucleotide encoding a fluorescent protein flanked by a first homology arm and a second homology arm.

2. The polynucleotide of claim 1, wherein the homology arms are 15-60 bases in length.

3. The polynucleotide of claim 1, wherein the homology arms are 25-45 bases in length.

4. The polynucleotide of claim 1, wherein the homology arms are 30-40 bases in length.

5. A double-stranded, linear donor polynucleotide comprising a polynucleotide encoding a fluorescent protein flanked by a first homology arm and a second homology arm, wherein the first and second homology arms are between 30-35 bases in length.

6. A double-stranded, linear donor polynucleotide comprising a template polynucleotide encoding an edit flanked by an intervening sequence and two homology arms.

7. The polynucleotide of claim 6, wherein the homology arms are 15-60 bases in length.

8. The polynucleotide of claim 6, wherein the homology arms are 25-45 bases in length.

9. The polynucleotide of claim 6, wherein the homology arms are 30-40 bases in length.

10. The polynucleotide of claim 6, wherein the template polynucleotide is up to 1 kb in length.

11. The polynucleotide of claim 6, wherein the template polynucleotide comprises a sequence designed to change at least one nucleotide base within 30 bases of a double-stranded break (DSB) of a target nucleic acid.

12. The polynucleotide of claim 11, wherein the template polynucleotide further comprises a restriction enzyme site.

13. A double-stranded, linear donor polynucleotide comprising a template polynucleotide flanked by a first homology arm and a second homology arm, wherein the homology arms are between 30-35 bases in length.

14. The polynucleotide of claim 14, wherein the template polynucleotide is up to 1 kb in length.

15. The polynucleotide of claim 14, wherein the template polynucleotide comprises a sequence designed to change at least one nucleotide base within 30 bases of a DSB of a target nucleic acid.

16. The polynucleotide of claim 15, wherein the template polynucleotide further comprises a restriction enzyme site.

17. A method comprising the step of performing a clustered regularly interspaced short palindromic repeats (CRISPR)-based technique using a double-stranded, linear donor polynucleotide of claim 6 as the donor polynucleotide.

18. A method comprising injecting into a target cell a composition comprising (a) an RNA-guided DNA endonuclease; (b) a guide RNA; and (c) a double-stranded, linear donor polynucleotide of claim 6.
